SEQUENCE LISTING (2 sequences)

SEQ ID NO: 1 - DNA, 16 bp, Artificial Sequence
  Synthetic sequence
  modified_base (1)..(1): gm
  modified_base (2)..(2): 2′-fluoro-deoxy-cytidine ribonucleotide
  modified_base (2)..(2): 5′-phosphorothioate
  modified_base (3)..(3): 2′-O-methyl-deoxy-adenosine ribonucleotide
  modified_base (3)..(3): 5′-phosphorothioate
  modified_base (4)..(4): 2′-fluoro-deoxy-guanosine ribonucleotide
  modified_base (5)..(5): 2′-fluoro-deoxy-uridine ribonucleotide
  modified_base (6)..(6): 2′-fluoro-deoxy-adenosine ribonucleotide
  modified_base (7)..(7): um
  modified_base (8)..(8): 2′-fluoro-deoxy-guanosine ribonucleotide
  modified_base (9)..(9): um
  modified_base (10)..(10): 2′-fluoro-deoxy-uridine ribonucleotide
  modified_base (11)..(11): gm
  modified_base (12)..(12): 2′-fluoro-deoxy-adenosine ribonucleotide
  modified_base (13)..(13): um
  modified_base (14)..(14): 2′-fluoro-deoxy-guanosine ribonucleotide
  modified_base (15)..(15): gm
  modified_base (16)..(16): 2′-fluoro-deoxy-adenosine ribonucleotide
gcaguauguugaugga

SEQ ID NO: 2 - RNA, 16 nt, Artificial Sequence
  Synthetic sequence
  modified_base (1)..(1): um
  modified_base (2)..(2): gm
  modified_base (15)..(15): cm
  misc_feature (15)..(15): 5′-phosphorothioate
  modified_base (16)..(16): 2′-O-methyl-deoxy-adenosine ribonucleotide
  misc_feature (16)..(16): 5′-phosphorothioate
ugugacuuccagacca

(19) United States
(12) Patent Application Publication
(10) Pub. No.: US 2024/0335551 A1
(43) Pub. Date: Oct. 10, 2024

(54) FUNCTIONAL MOIETIES AND THEIR USES AND SYNTHETIC PREPARATION

(71) Applicant: OliX US, Inc., San Diego, CA (US)

(72) Inventors: Dongwon Shin, San Diego, CA (US); Namho Kim, San Diego, CA (US); Yi-An Lin, San Diego, CA (US); Raymond Emehiser, Santee, CA (US)

(21) Appl. No.: 18/562,731

(22) PCT Filed: May 20, 2022
(86) PCT No.: PCT/US2022/030251

§ 371 Date: Nov. 20, 2023

Related U.S. Application Data
(60) Provisional application No. 63/191,040, filed on May 20, 2021.

(51) Int. Cl. A61K 47/65; A61K 47/54

(52) U.S. Cl. CPC A61K 47/65; A61K 47/549

(57) ABSTRACT

Provided herein are functional moieties, functionalized compounds and macromolecules, their preparation, and uses thereof.

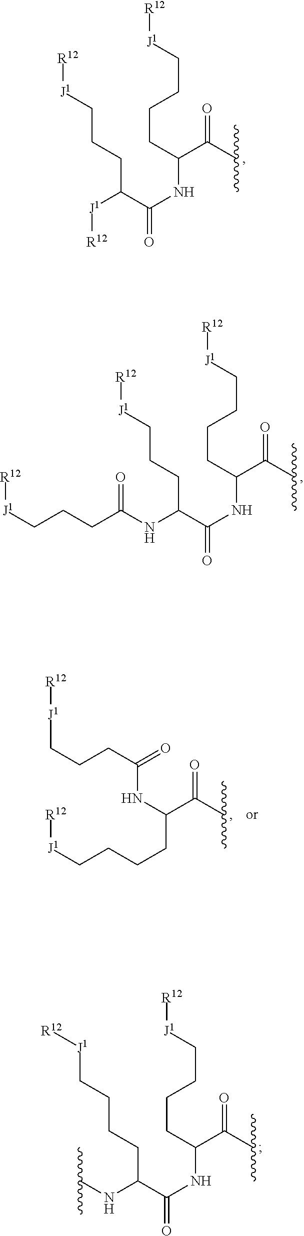

### RELATED APPLICATIONS

This application claims priority of U.S. Provisional Patent Application No. 63/191,040, filed May 20, 2021, the entire contents of which are incorporated herein by reference.

### SEQUENCE LISTING

This application contains a sequence listing having the filename 0817444-00006_ST25.txt, which is 4,096 bytes in size, and was created on May 6, 2021. The entire content of this sequence listing is incorporated herein by reference.

### BACKGROUND

In vivo, many macromolecules are decorated with a variety of functional groups that, for example, tag the macromolecule for recognition by a particular enzyme, impart desirable characteristics to the macromolecule, or make the macromolecule easy to manipulate or visualize. For example, proteins may be functionalized by enzymatic post-translational modification including phosphorylation, glycosylation, ubiquitination, sumoylation, ribosylation, citrullination, nitrosylation, methylation, acetylation, lipidation, as well as other types of modifications. Oligonucleotides such as deoxyribonucleic acids (DNA) may undergo modifications including methylation or hydroxymethylation, and ribonucleic acids (RNA) may undergo post-transcriptional modification in a cell, including polyadenylation, capping the 5′-end of the RNA molecule, RNA-editing, methylation, or demethylation, as well as other types of modifications.

Solid support synthesis is a useful tool to prepare macromolecules by sequentially iterating through coupling cycles. Typically, a machine is used to perform each cycle, which may include a number of chemical steps, in order to improve overall yield of a final desired product. Solid support synthesis has been used successfully with peptides, oligonucleotides, and oligomeric carbohydrates, as well as other types of macromolecules.

The ability to functionalize compounds or macromolecules in a controlled and specific manner is necessary to be able to assess the utility of such functionalized compounds or macromolecules in a therapeutic application. Thus, there remains a need to develop tools to functionalize compounds or macromolecules using solid support synthesis, and to develop functional moieties compatible with solid support synthesis that may impart desirable characteristics to a compound or macromolecule.

Thus, provided herein are functional moieties useful in solid support synthesis, methods of preparing such moieties, compounds or macromolecules functionalized with such moieties, methods of preparing such functionalize moieties, and building blocks for solid support synthesis that include such moieties.

### SUMMARY

Provided herein are functional moieties, functionalized compounds and macromolecules, their preparation, and uses thereof.

In some embodiments, provided herein are compounds comprising one or more of the following formulae:

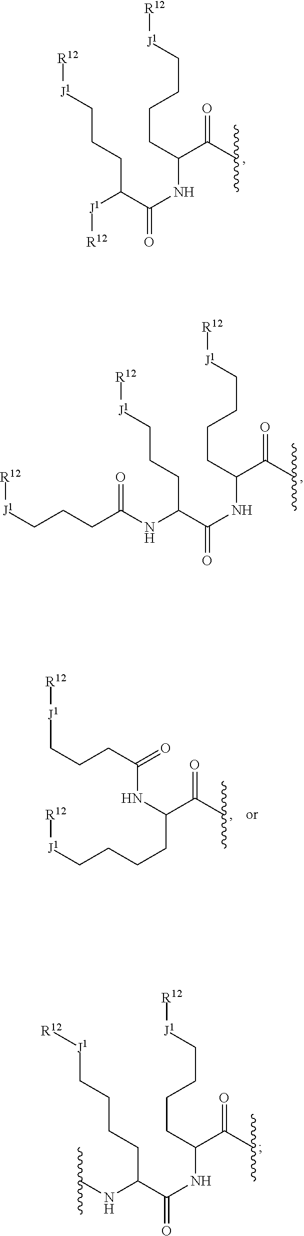

or a salt thereof.

In some embodiments, provided herein are compounds comprising the following formula:

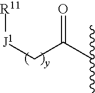

or a salt thereof.

In some embodiments, provided herein are compounds comprising the following formula:

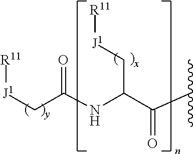

or a salt thereof.

In some embodiments, provided herein are compounds comprising the following formula:

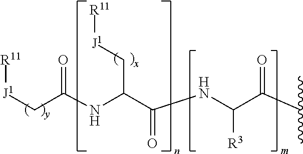

or a salt thereof.

In some embodiments, provided herein are compounds comprising the following formula:

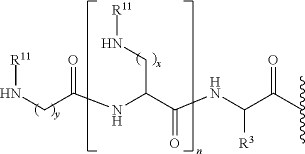

or a salt thereof.

In some embodiments, provided herein are compounds comprising the following formula:

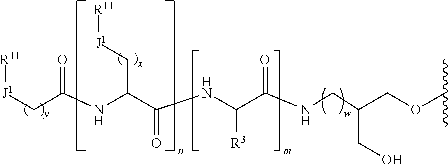

or a salt thereof.

In some embodiments, provided herein are compounds comprising the following formula:

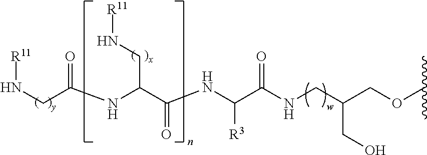

or a salt thereof.

In some embodiments, provided herein are compounds comprising the following formula:

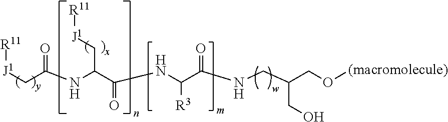

or a salt thereof.

In some embodiments, provided herein are compounds comprising the following formula:

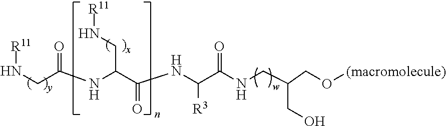

or a salt thereof.

In some embodiments, provided herein are compounds comprising the following formula:

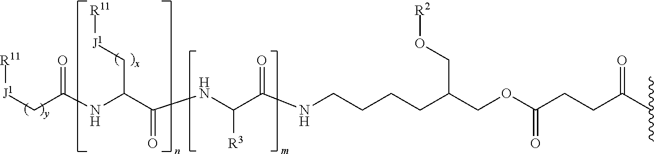

or a salt thereof.

In some embodiments, provided herein are compounds comprising the following formula:

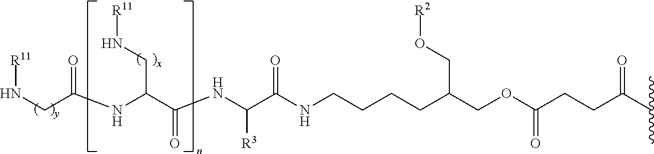

or a salt thereof.

In some embodiments, provided herein are compounds comprising the following formula:

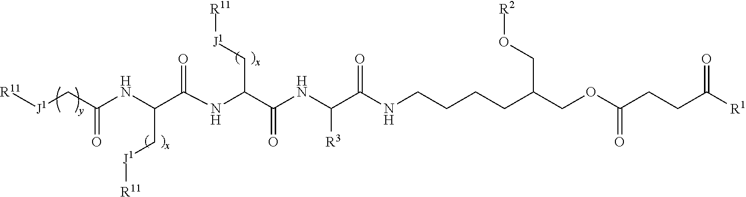

or a salt thereof.

In some embodiments, provided herein are compounds comprising the following formula:

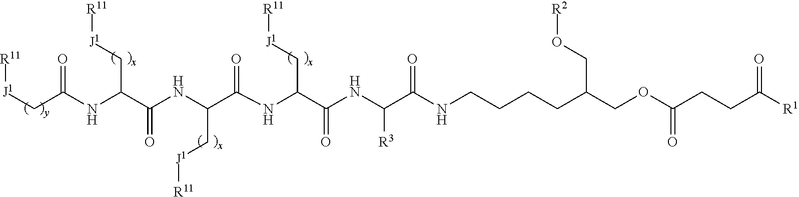

or a salt thereof.

In some embodiments, provided herein are compounds comprising the following formula:

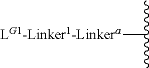

or a salt thereof.

In some embodiments, provided herein are compounds comprising the following formula:

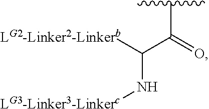

or a salt thereof.

In some embodiments, provided herein are compounds comprising the following formula:

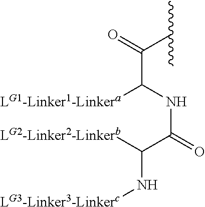

or a salt thereof.

In some embodiments, provided herein are compounds comprising the following formula:

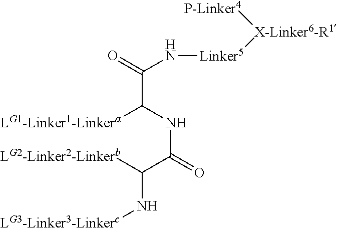

or a salt thereof.

In some embodiments, provided herein are compounds comprising the following formula:

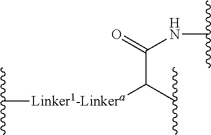

or a salt thereof.

In some embodiments, provided herein are compounds comprising the following formula:

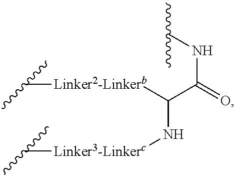

or a salt thereof.

### BRIEF DESCRIPTION OF THE DRAWINGS

FIG. 1 shows a general synthetic scheme useful in preparing the functionalized compounds described herein.

FIG. 2 shows another general synthetic scheme useful in preparing the functionalized compounds described herein.

FIG. 3 shows still another general synthetic scheme useful in preparing the functionalized compounds described herein.

FIG. 4 shows a chemical structure for the compound of Examples 1 and 3.

FIG. 5 shows a chemical structure for the compound of Example 2.

FIG. 6 shows a chemical structure for the compound of Example 4.

FIG. 7 shows a chemical structure for the compound of Example 5.

FIG. 8 shows a chemical structure for the compound of Example 6.

FIG. 9 shows a chemical structure for the compound of Example 7.

FIG. 10 shows a chemical structure for the compound of Example 8.

FIG. 11 shows a chemical structure for the compound of Example 9.

FIG. 12 shows a chemical structure for the compound of Example 10.

FIG. 13 shows a chemical structure for the compound of Example 11.

FIG. 14 shows chemical structures for certain chemical moieties referred to herein.

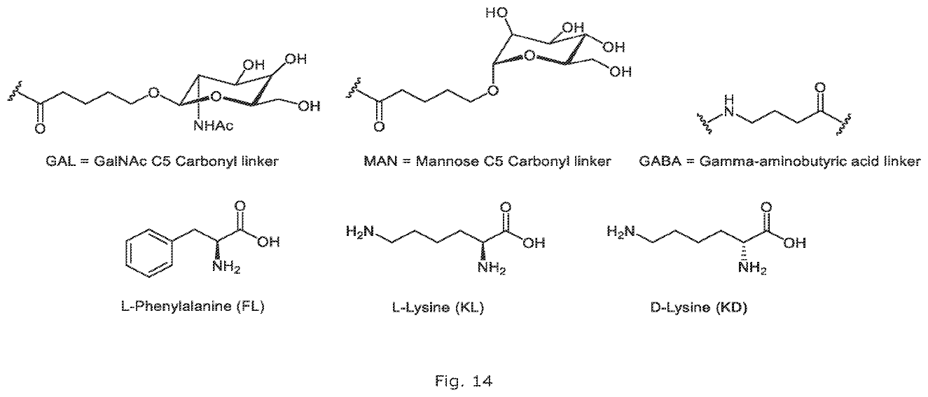

FIG. 15 shows chemical structures for certain nucleotide moieties referred to herein.

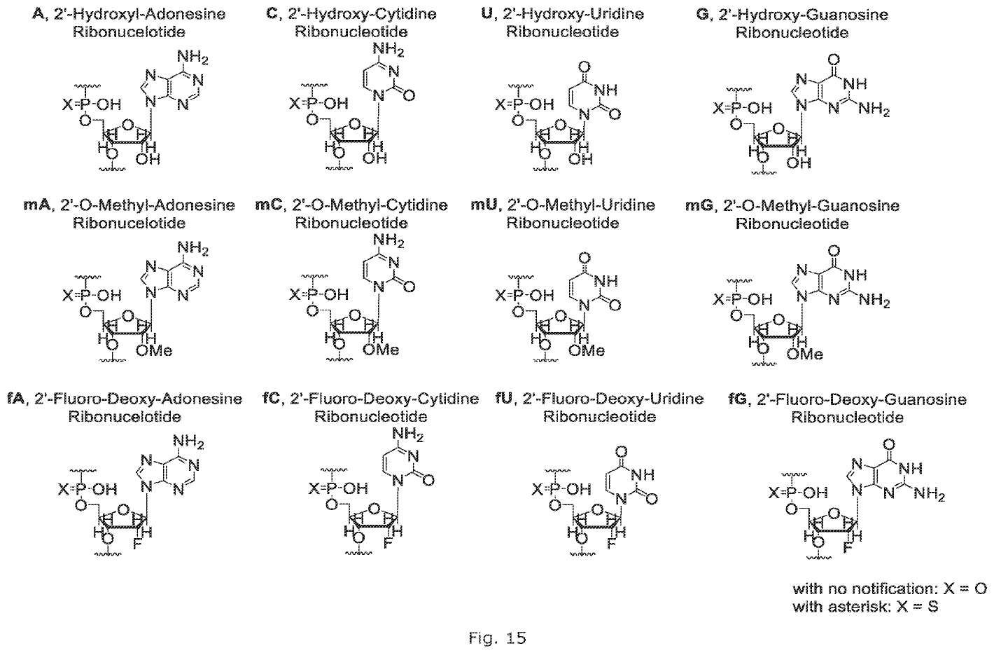

### DETAILED DESCRIPTION

Macromolecules have important therapeutic applications in the pharmaceutical and medicinal fields. For example, oligonucleotides can be applied to silence genes by inhibiting the translation process, which inhibits formation of proteins responsible for a particular disease. Thus, the specific role of gene-silencing oligonucleotides has been one alternative to traditional small molecules that inhibit the function of the protein linked to the diseases. RNA therapeutics provide the potential for the precise treatment of genetic diseases. For example, siRNA, antisense RNA, and micro-RNA are types of oligonucleotides that prevent the formation of proteins by gene-silencing.

Unmodified macromolecules, including oligonucleotides, generally possess poor therapeutic properties. Although the conceptual applications of oligonucleotides and oligonucleotide analogs have been advanced in the last several decades, therapeutic applications have been limited due to the low profiles of pharmaceutical properties, e.g., stability, specificity, affinity, and moderate to high profiles in toxicity leading to off-target effects.

To overcome these, and other, limitations, and to introduce improved stability and delivery of compounds or macromolecules, there is a need for a fast and tunable approach to introduce bioconjugate linkers to compounds or macromolecules. Such modifications may impart increased stability, binding affinity, specificity, improved cellular uptake or subcellular trafficking, or low profile of toxicity and off-target effects, or a combination thereof, to such functionalized compounds or macromolecules. There is a paucity, and therefore a need, for bioconjugation linkers containing various functional groups to improve the pharmaceutical properties of macromolecules that may be so conjugated, the bioconjugation linkers in some embodiments being themselves linked in a chemically-cleavable, thermally cleavable, or photo-cleavable way to a solid support.

Thus, in some embodiments, provided herein are compounds, comprising one or more of the following formulae:

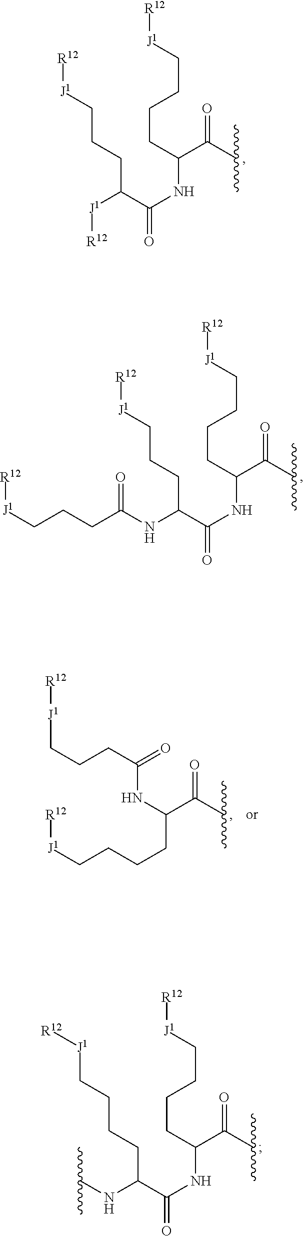

or a salt thereof,
wherein
each R¹² is, independently, selected from H or —C(O)(CH₂)_{z}-(ligand);
each z is, independently, selected from 1, 2, 3, 4, 5, or 6; and
each ligand is, independently, selected from a lipophilic group, a protein, a peptide, an oligosaccharide, a nucleic acid, a polymer, a carbohydrate, or a lipid.

In some embodiments, provided herein are compounds comprising a macromolecule covalently linked to one or more, independently, of the following formula:

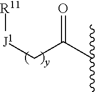

or a salt thereof,
wherein
R¹¹ is selected from H, —C(O)(CH₂)_{z}-(ligand),

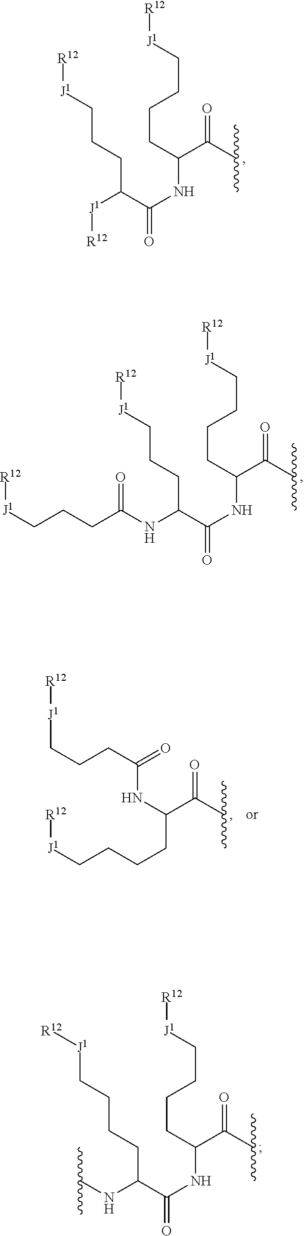

each R¹² is, independently, selected from H or —C(O)(CH₂)_{z}-(ligand);
each y is, independently, selected from 1, 2, 3, 4, 5, or 6;
each z is, independently, selected from 1, 2, 3, 4, 5, or 6; and
each ligand is, independently, selected from a lipophilic group, a protein, a peptide, an oligosaccharide, a nucleic acid, a polymer, a carbohydrate, or a lipid.

In some embodiments of the compounds provided herein, the compound comprises a macromolecule covalently linked to one or more, independently, of the following formula:

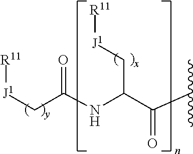

or a salt thereof,
wherein
each R¹¹ is, independently, selected from H, —C(O)(CH₂)_{z}-(ligand),

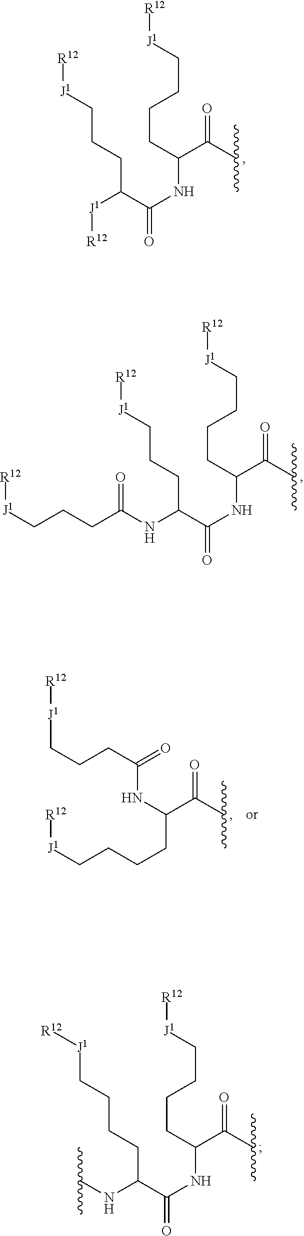

each x is, independently, selected from 1, 2, 3, 4, 5, or 6;
each y is, independently, selected from 1, 2, 3, 4, 5, or 6;
each z is, independently, selected from 1, 2, 3, 4, 5, or 6;
n is 0, 1, 2, 3, 4, 5, or 6; and
each ligand is, independently, selected from a lipophilic group, a protein, a peptide, an oligosaccharide, a nucleic acid, a polymer, a carbohydrate, or a lipid.

In some embodiments of the compounds provided herein, the compound comprises a macromolecule covalently linked to one or more, independently, of the following formula:

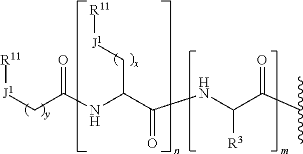

or a salt thereof,
wherein
R³ is —(C₀₋₃ alkylene)-(C₃₋₈ cycloalkyl), —(C₀₋₃ alkylene)-(C₃₋₈ heterocycloalkyl), —(C₁₋₃ alkylene)-(C₆₋₁₀ aryl), —(C₁₋₃ alkylene)-(C₂₋₁₀ heteroaryl), wherein the aryl or heteroaryl is optionally substituted by one or two functional groups selected, independently, from OH, F, Cl, Br, I, O—C₁₋₃ alkyl, C₁₋₃ alkyl, C₁₋₃ alkenyl, C₁₋₃ alkynyl, C₁₋₃ haloalkyl, COOH, or NH₂;
each R¹¹ is, independently, selected from H, —C(O)(CH₂)_{z}-(ligand),

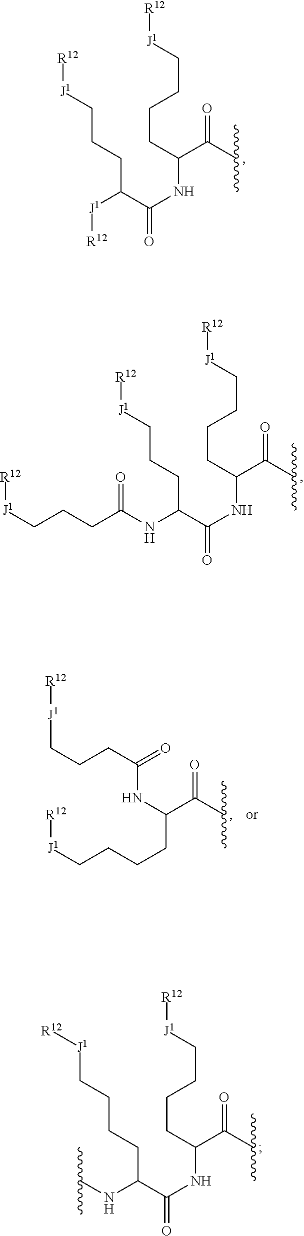

each x is, independently, selected from 1, 2, 3, 4, 5, or 6;
each y is, independently, selected from 1, 2, 3, 4, 5, or 6;
each z is, independently, selected from 1, 2, 3, 4, 5, or 6;
m is 0, 1, 2, 3, 4, 5, or 6;
n is 0, 1, 2, 3, 4, 5, or 6; and
each ligand is, independently, selected from a lipophilic group, a protein, a peptide, an oligosaccharide, a nucleic acid, a polymer, a carbohydrate, or a lipid.

In some embodiments of the compounds provided herein, the compound comprises a macromolecule covalently linked to one or more, independently, of the following formula:

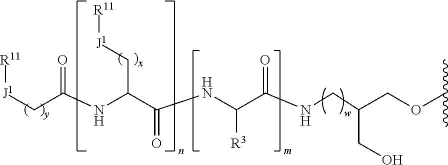

or a salt thereof,
wherein
R³ is —(C₀₋₃ alkylene)-(C₃₋₈ cycloalkyl), —(C₀₋₃ alkylene)-(C₃₋₈ heterocycloalkyl), —(C₁₋₃ alkylene)-(C₆₋₁₀ aryl), —(C₁₋₃ alkylene)-(C₂₋₁₀ heteroaryl), wherein the aryl or heteroaryl is optionally substituted by one or two functional groups selected, independently, from OH, F, Cl, Br, I, O—C₁₋₃ alkyl, C₁₋₃ alkyl, C₁₋₃ alkenyl, C₁₋₃ alkynyl, C₁₋₃ haloalkyl, COOH, or NH₂;
each R¹¹ is, independently, selected from H, —C(O)(CH₂)_{z}-(ligand),

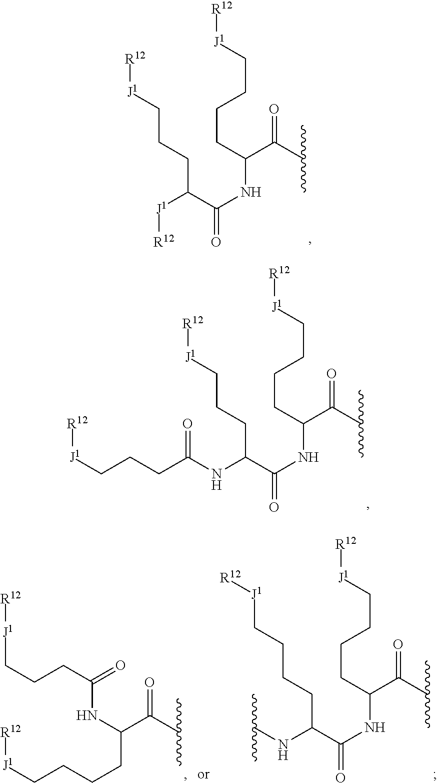

each x is, independently, selected from 1, 2, 3, 4, 5, or 6;
each y is, independently, selected from 1, 2, 3, 4, 5, or 6;
each z is, independently, selected from 1, 2, 3, 4, 5, or 6;
each w is, independently, selected from 1, 2, 3, 4, 5, or 6;
m is 0, 1, 2, 3, 4, 5, or 6;
n is 0, 1, 2, 3, 4, 5, or 6; and
each ligand is, independently, selected from a lipophilic group, a protein, a peptide, an oligosaccharide, a nucleic acid, a polymer, a carbohydrate, or a lipid.

In some embodiments of the compounds provided herein, the compound comprises the following formula:

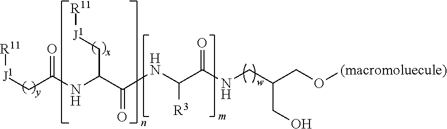

or a salt thereof, the variables being as defined herein.

In some embodiments of the compounds provided herein, the compound comprises a macromolecule covalently linked to one or more, independently, of the following formula:

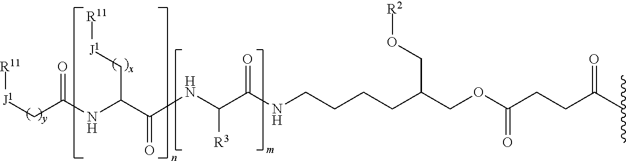

or a salt thereof,
wherein
R² is a thermolytic, photolytic, tris(2-carboxyethyl) phosphine-labile, acid-labile, or base-labile moiety;
R³ is —(C₀₋₃ alkylene)-(C₃₋₈ cycloalkyl), —(C₀₋₃ alkylene)-(C₃₋₈ heterocycloalkyl), —(C₁₋₃ alkylene)-(C₆₋₁₀ aryl), —(C₁₋₃ alkylene)-(C₂₋₁₀ heteroaryl), wherein the aryl or heteroaryl is optionally substituted by one or two functional groups selected, independently, from OH, F, Cl, Br, I, O—C₁₋₃ alkyl, C₁₋₃ alkyl, C₁₋₃ alkenyl, C₁₋₃ alkynyl, C₁₋₃ haloalkyl, COOH, or NH₂;
each R¹¹ is, independently, selected from H, —C(O)(CH₂)_{z}-(ligand),

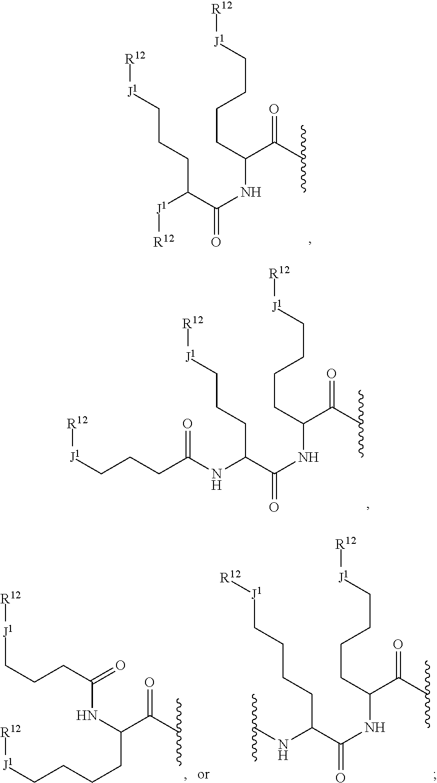

each x is, independently, selected from 1, 2, 3, 4, 5, or 6;
each y is, independently, selected from 1, 2, 3, 4, 5, or 6;
each z is, independently, selected from 1, 2, 3, 4, 5, or 6;
m is 0, 1, 2, 3, 4, 5, or 6;
n is 0, 1, 2, 3, 4, 5, or 6; and
each ligand is, independently, selected from a lipophilic group, a protein, a peptide, an oligosaccharide, a nucleic acid, a polymer, a carbohydrate, or a lipid.

In some embodiments of the compounds provided herein, the compound comprises the following formula:

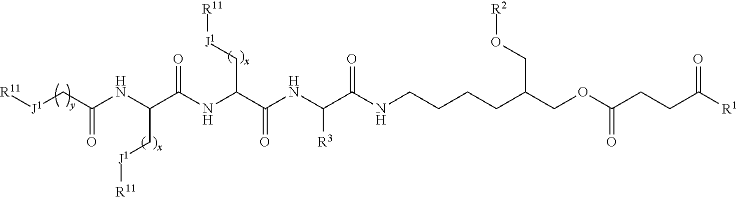

or a salt thereof,
wherein
R¹ is a solid support.

In some embodiments of the compounds provided herein, the compound comprises the following formula:

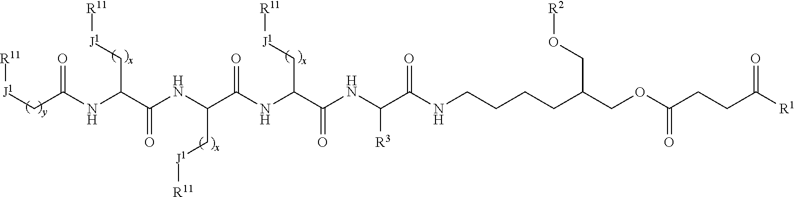

or a salt thereof,
wherein
R¹ is a solid support.

In some embodiments of the formulae provided herein,
each J¹ is, independently, N(H) or C(O); and
each R¹¹ or R¹² is, independently, H, OH, —C(O)(CH₂)_{z}-(ligand), —N(H)(CH₂)_{z}-(ligand), or —O—(CH₂)_{z}-(ligand).

In some embodiments of the formulae provided herein,
each J¹ is, independently, N(H); and
each R¹¹ or R¹² is, independently, H or —C(O)(CH₂)_{z}-(ligand).

In some embodiments of the formulae provided herein,
each J¹ is, independently, C(O); and
each R¹¹ or R¹² is, independently, OH or —N(H)(CH₂)_{z}-(ligand).

In some embodiments of the formulae provided herein, each independent instance of

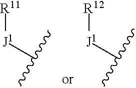

may be replaced with

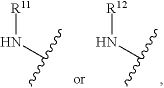

respectively.

In some embodiments of the formulae provided herein, each independent instance of

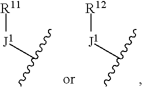

may be replaced with

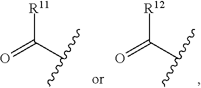

respectively.

As used herein, the term “hetero” refers to one or more heteroatoms selected from N, O, or S. In some embodiments, “hetero” refers to one, two, three, or four heteroatoms, each, independently, selected from N, O, or S.

In some embodiments of the compounds provided herein, the macromolecule is a peptide, a protein, an oligosaccharide, an oligomer (e.g., an oligonucleotide), or a solid support. In some embodiments of the compounds provided herein, the macromolecule is a polymer or oligomer comprising one or more monomer units, each monomer unit comprising a position for a moiety independently selected from 1) nucleobases (e.g., canonical nucleobases, e.g., A, G, C, T, U) capable of orthogonal pairing, 2) nucleobase analogs (e.g., non-canonical nucleobases, e.g., A, G, C, T, U mimics, e.g., hypoxanthine, xanthine, 7-methylguanine, 5,6-dihydrouracil, 5-methylcytosine, 5-hydroxymethylcytosine, purine, pyrimidine, 2,6-diaminopurine, pseudouracil, isoguanine, isocystosine, aminoallyl-uracil, 2-amino-6-(2-thienyl) purine, pyrrole-2-carbaldehyde, thiouracil, 5-bromocytosine, 5-bromouracil, 5-iodouracil, 6-thioguanine, etc.) capable of orthogonal pairing, or 3) the position on the monomer unit is abasic (i.e., lacking a nucleobase or nucleobase analog). In some embodiments, each occurrence of the nucleobase(s) of the polymers, oligomers, or oligonucleotides herein comprises, independently, a C₃₋₆ heterocyclic ring (e.g, a C₃₋₆ heterocyclic ring independently at each occurrence selected from pyridine, pyrimidine, triazinane, purine, and deaza-purine), wherein the nucleobase is capable of orthogonal pairing. In some embodiments, the nucleobase may be functionalized with a chemical protecting-group to prevent unwanted side-reactions during synthetic preparative sequence(s). In some embodiments of the compounds provided herein, the macromolecule comprises an oligonucleotide. In some embodiments, the oligonucleotide comprises ribonucleic acid, deoxyribonucleic acid, or both. In some embodiments, the oligonucleotide comprises an RNAi, mRNA, miRNA, siRNA, snoRNA, saRNA, or piRNA oligonucleotide. In some embodiments the oligonucleotide comprises single-stranded oligonucleotide. In some embodiments, the oligonucleotide comprises double-stranded (“ds”) oligonucleotide. In some embodiments the oligonucleotide comprises dsRNA or dsDNA. In some embodiments, the oligonucleotide is 50 nucleotides (“nt”) in length or less, whether single-stranded or double-stranded. In some embodiments, the oligonucleotide is about 5-50 nt. 5-40 nt. 5-30 nt. 5-25 nt. 5-20 nt. 5-15 nt. 5-10 nt. 10-30 nt. 10-25 nt. 10-20 nt. 10-15 nt. 15-30 nt. 15-25 nt. 15-20 nt. 20-30 nt. 20-25 nt, about 5 nt. 10 nt. 15 nt. 20 nt. 25 nt. 30 nt. 40 nt, or 50 nt in length. In some embodiments, the oligonucleotide is about 14, 15, 16, 17, 18, 19, 20, 21, or 22 nt in length. In some embodiments, the recited oligonucleotide length or range refers to the recited length or range value+2 nt. In some embodiments, the polymers or oligomers provided herein may be described by a length of monomer unit synonymous with the above nucleotide length.

In some embodiments of the compounds provided herein, the one or more formulae comprised in the compound is: covalently connected, directly or indirectly, to 1, 2, 3, 4, 5, or 6 ligands; and covalently connected, directly or indirectly, to one or two macromolecules. In some embodiments of the compounds provided herein each ligand of the compound is, independently, selected from a lipophilic group, a protein, a peptide, an oligosaccharide, a nucleic acid, a polymer, a carbohydrate, or a lipid. In some embodiments, each ligand is, independently, selected from a mannose moiety, an N-acetylated galactosamine moiety, a tetra-acetylated mannose moiety or a tetra-acetylated galactosamine moiety. In some embodiments of the compounds provided herein, each macromolecule is, independently, selected from a peptide, a protein, an oligosaccharide, an oligomer (e.g., an oligonucleotide), or a solid support. In some embodiments, one macromolecule in the compound is a solid support. In some embodiments, one macromolecule in the compound is an oligomer, whether protected with protecting groups or deprotected from protecting groups comprising one or more monomer units, each monomer unit comprising a C₃₋₆ heterocyclic ring. In some embodiments, the oligomer in the compounds herein is an oligonucleotide. In some embodiments, the oligomer is an oligonucleotide selected from SEQ ID NO: 1 or SEQ ID NO:2.

In some embodiments, of the formulae provided herein. R² is a thermolytic, photolytic, tris(2-carboxyethyl) phosphine-labile, acid-labile, or base-labile moiety. In some embodiments of the formulae provided herein. R² is an acid-labile moiety. In some embodiments. R² is triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl.

Thermolytic moieties are protecting groups that can be removed by increasing temperature, i.e. above standard room temperature. In some embodiments, thermolytic, also referred to as thermolabile, moieties include thermolytic carbonates, and thermolytic carbamoyls. In some embodiments, thermolabile moieties are selected from, but not limited to, the protecting groups containing thermolabile 2-pyridy-moiety, carbamoyl moiety. 4-methylthio-1-butyl moiety. 4-hydroxy-1-butyl moiety, 4-phosphato/thiophosphato-1-butyl moiety, unsubstituted trityl, substituted trityl moiety (i.e, trimethoxytrityl, dimethoxytrityl, monomethoxytrityl, chlorotrityl), 2-cyanoethyl moiety, 2-(N-formyl-N-methyl)aminocthyl moiety, carbonate moiety, and (tert-butoxy)-1-ethyl moiety. In some embodiments, thermolytic refers to 2-pyridyl-aminoethyl carbonates or N-arylcarbamoyls. In some embodiments, thermolytic refers to 2-pyridyl-N-(2,4-difluorobenzyl)aminocthyl carbonate, N-(phenylsulfonyl) carbamoyl, monomethoxytrityl, dimethoxy trityl, or 2-(N-formyl-N-methyl)aminoethyl moieties.

In some embodiments, photolytic, also referred to as photolabile, photosensitive, photocleavable, or photoremovable, moieties are protecting groups that can be removed with light (whether ultraviolet or visible). As the cleavage of photolabile protecting groups does not require any chemical reagents, the photocleavage is relatively clean, casy, and generally does not harm the parent molecular structure. Photolabile protecting groups are selected from, but not limited to, nitrobenzyl-based photolabile protecting groups such as those protecting groups containing 2,6-dinitrobenzyl moiety, 2-cyano-6-nitrobenzyl, 2-nitroveratryl moiety or 6-nitropiperonulmethyl moiety, carbonyl-based photolabile protecting groups such as those protecting groups containing 3′,5′-dimethoxybenzoin (DMB) substituent on the carbonyl's alpha-carbon, phenacyl moiety, o-hydroxyphenacyl moiety, m-hydroxyphenacyl moiety, p-hydroxyphenacyl moiety, and benzyl-based photolabile protecting groups such as those protecting groups containing benzyl, naphthalenyl, anthracenyl, phenanthrenyl, pyrenyl, and perylenyl core and their derivatives.

In some embodiments, the compounds described herein include TCEP-sensitive moieties. Tris(2-carboxyethyl) phosphine (TCEP) is used for the deprotection of TCEP-sensitive protecting groups, which are selected from, but not limited to, those protecting group containing methylene azide moiety and modified methylene azide moiety.

In some embodiments of the compounds provided herein, the solid support comprises a long-chain alkyl amino linker. In some embodiments, the long-chain alkyl amino linker is a C₅₋₄₀ alkyl-amino linker.

In some embodiments of the compounds provided herein, the macromolecule refers to a solid support.

In some embodiments, each R³ is, independently, selected from

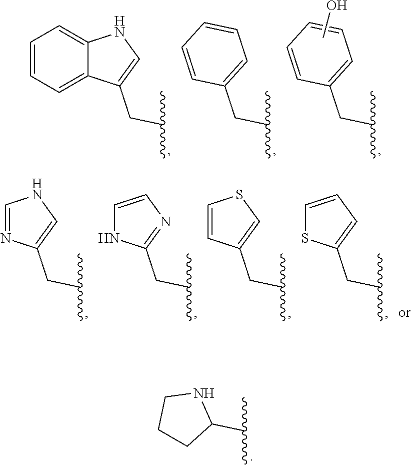

In some embodiments, each R³ is, independently, selected from

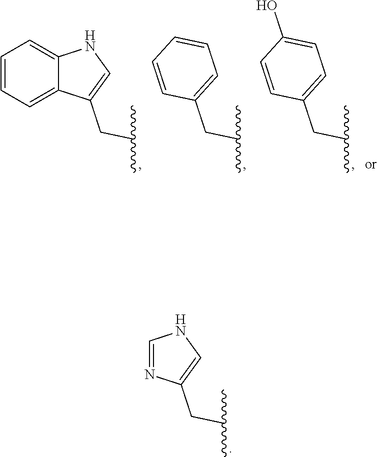

In some embodiments, each R³ is benzyl.

In some embodiments, m is 0, 1, 2, or 3. In some embodiments, m is 1. In some embodiments, m is 0.

Some of the compounds provided herein include peptide backbone linkages including one or more stereocenters. In some embodiments, each stereocenter of the peptide backbone is, independently, racemic, D-, or L-.

In some embodiments of the compounds provided herein, each x is 3 or 4, and each n is 2 or 3.

In some embodiments of the compounds provided herein, each R¹¹ and R¹² is H.

In some embodiments of the compounds provided herein, each ligand is, independently, selected from a mannose moiety, an N-acetylated galactosamine moiety, a tetra-acetylated mannose moiety or a tetra-acetylated galactosamine moiety. In some embodiments of the compounds provided herein, each ligand is, independently, selected from a mannose moiety or an N-acetylated galactosamine moiety.

In some embodiments of the compounds provided herein, each x is the same, and each ligand is the same.

In some embodiments of the compounds provided herein, each z is 4.

In some embodiments, each z is, independently, 3 or 4, and each ligand is, independently, a mannose moiety, an N-acetylated galactosamine moiety, a tetra-acetylated mannose moiety or a tetra-acetylated galactosamine moiety.

In some embodiments, the compound is a macromolecule functionalized with one or more of the formulae provided herein.

In some embodiments, the compound is Example 1, Example 2, Example 3, Example 4, Example 5, Example 6, Example 7, Example 8, Example 9, Example 10, Example 11, Example 12, or Example 13, or a salt thereof.

In some embodiments of the compounds provided herein, the compound comprises one of the following formulae:

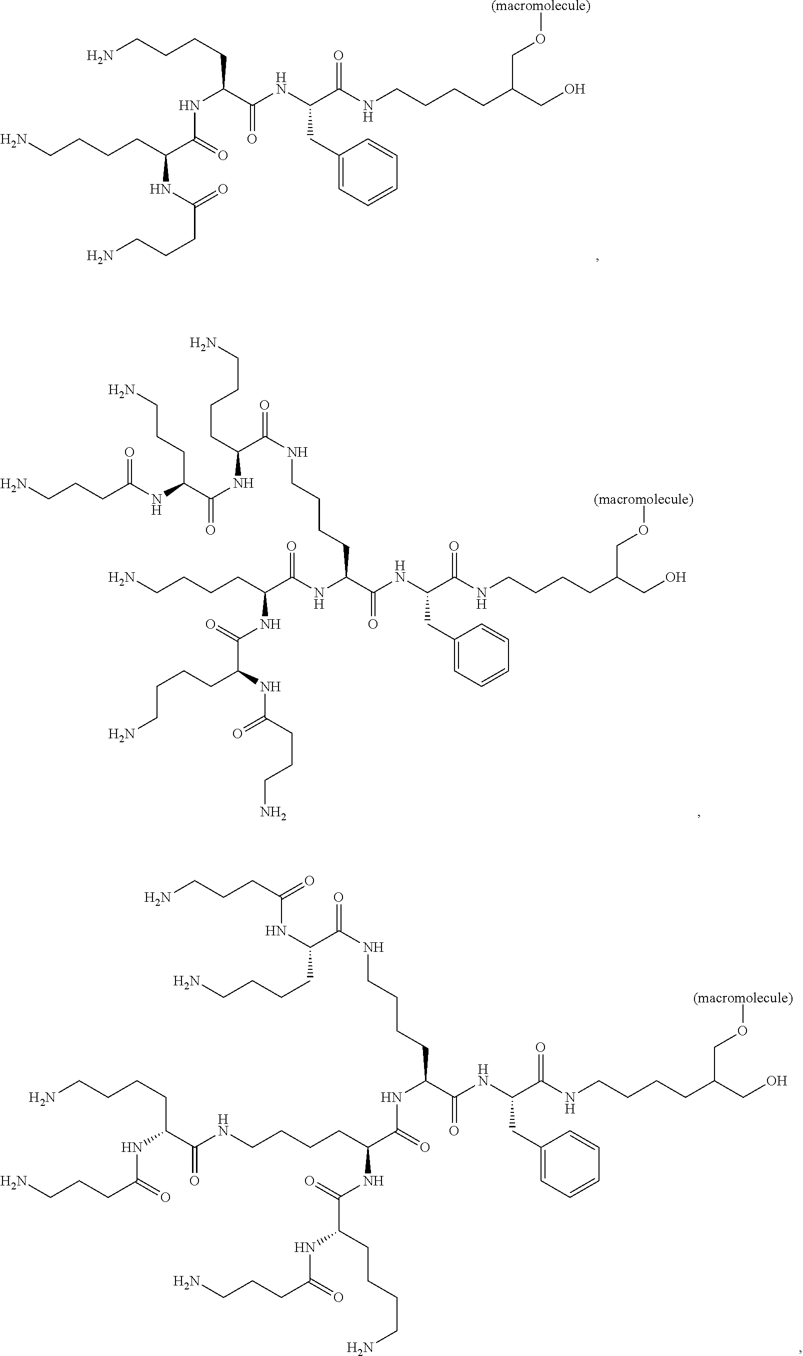

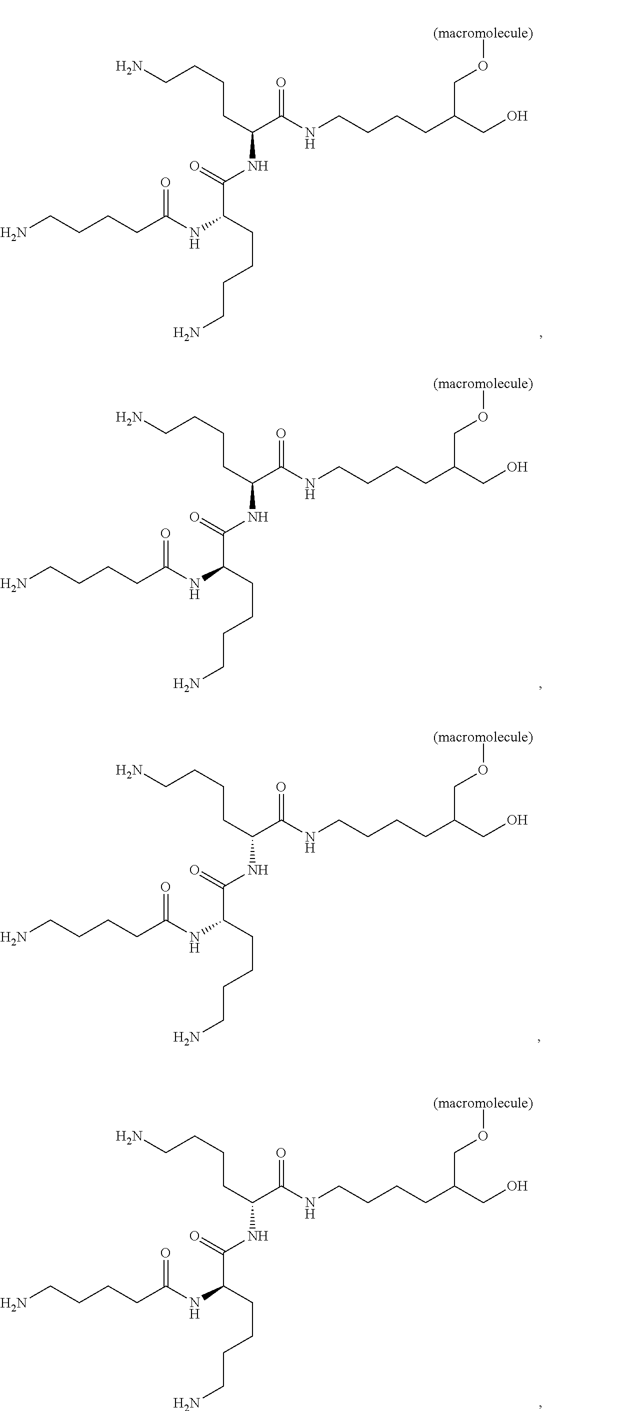

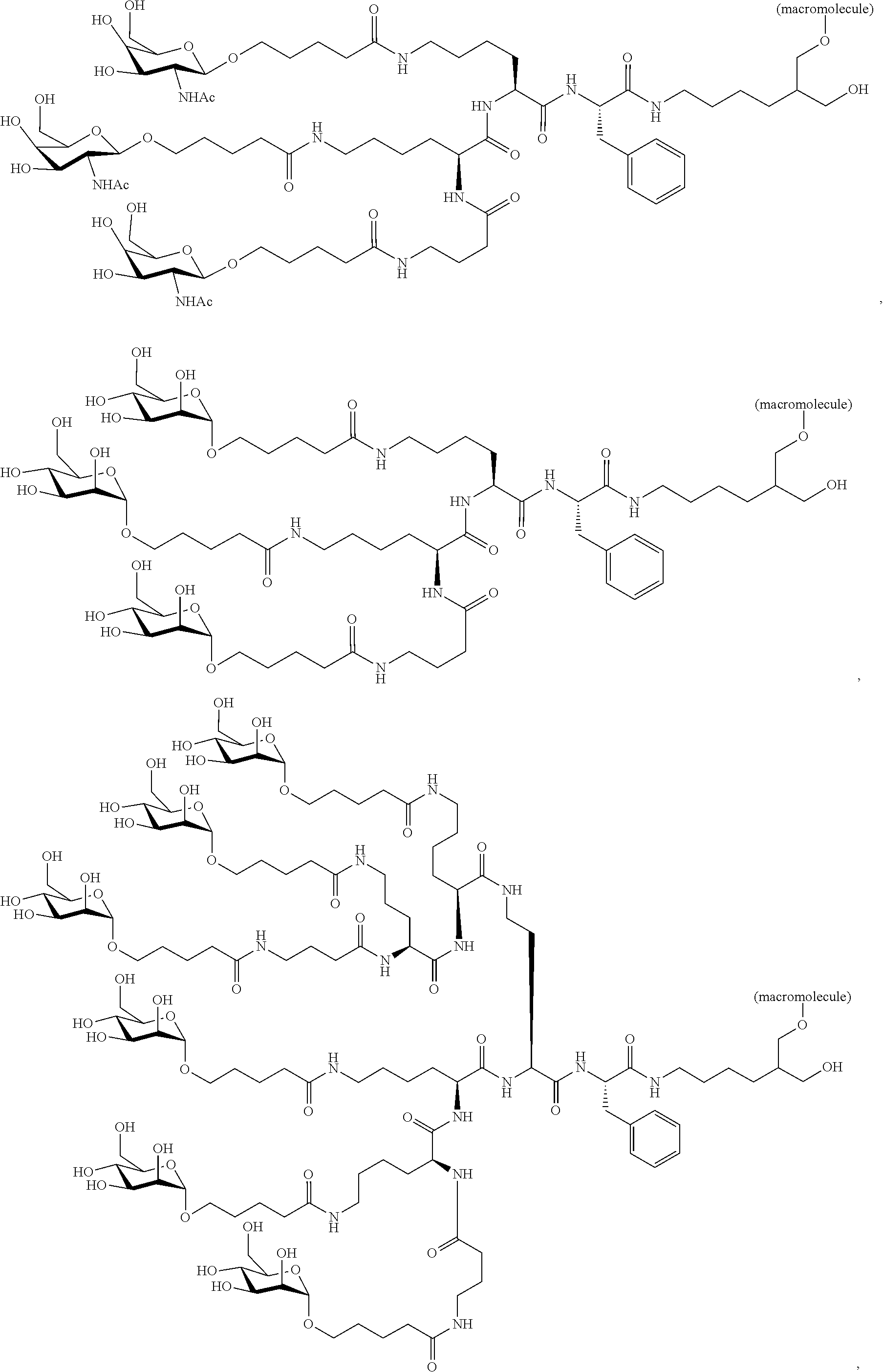

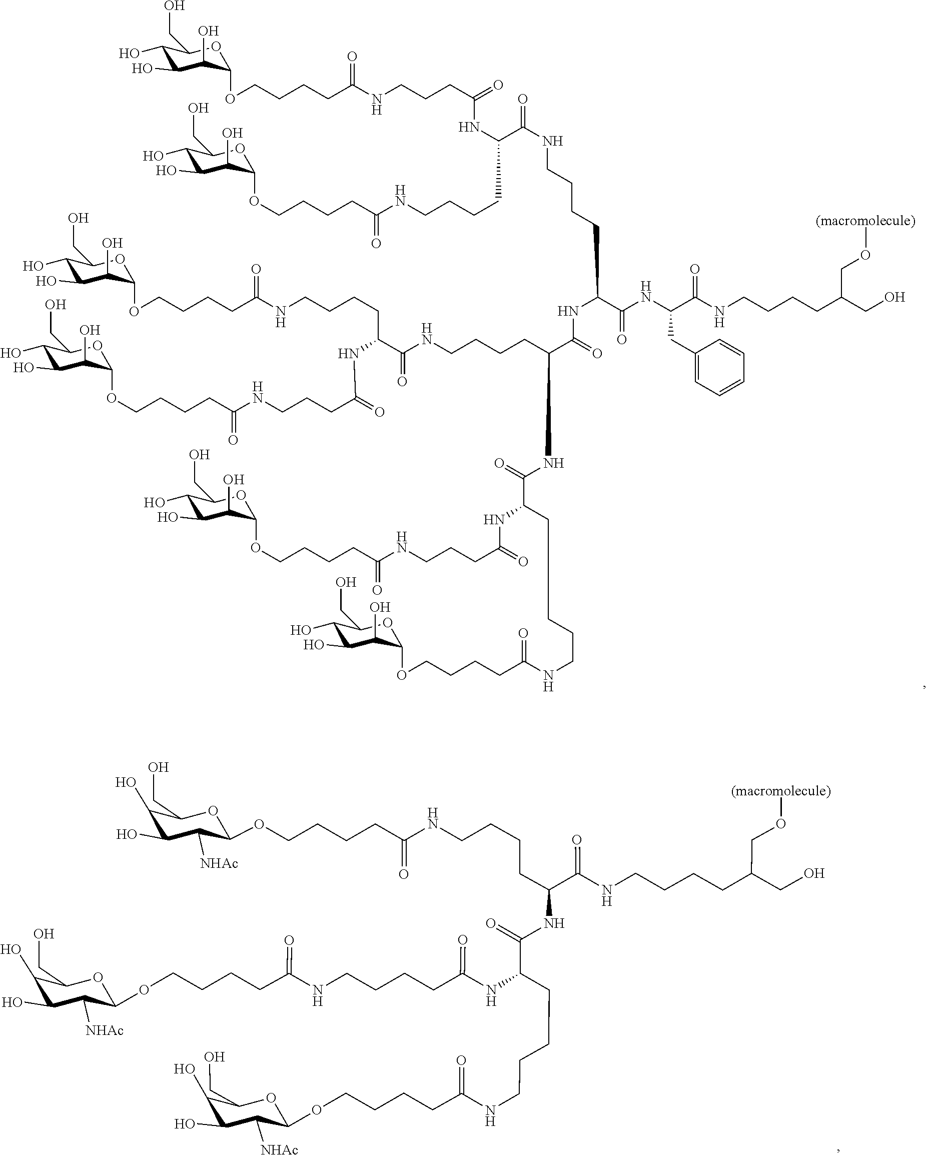

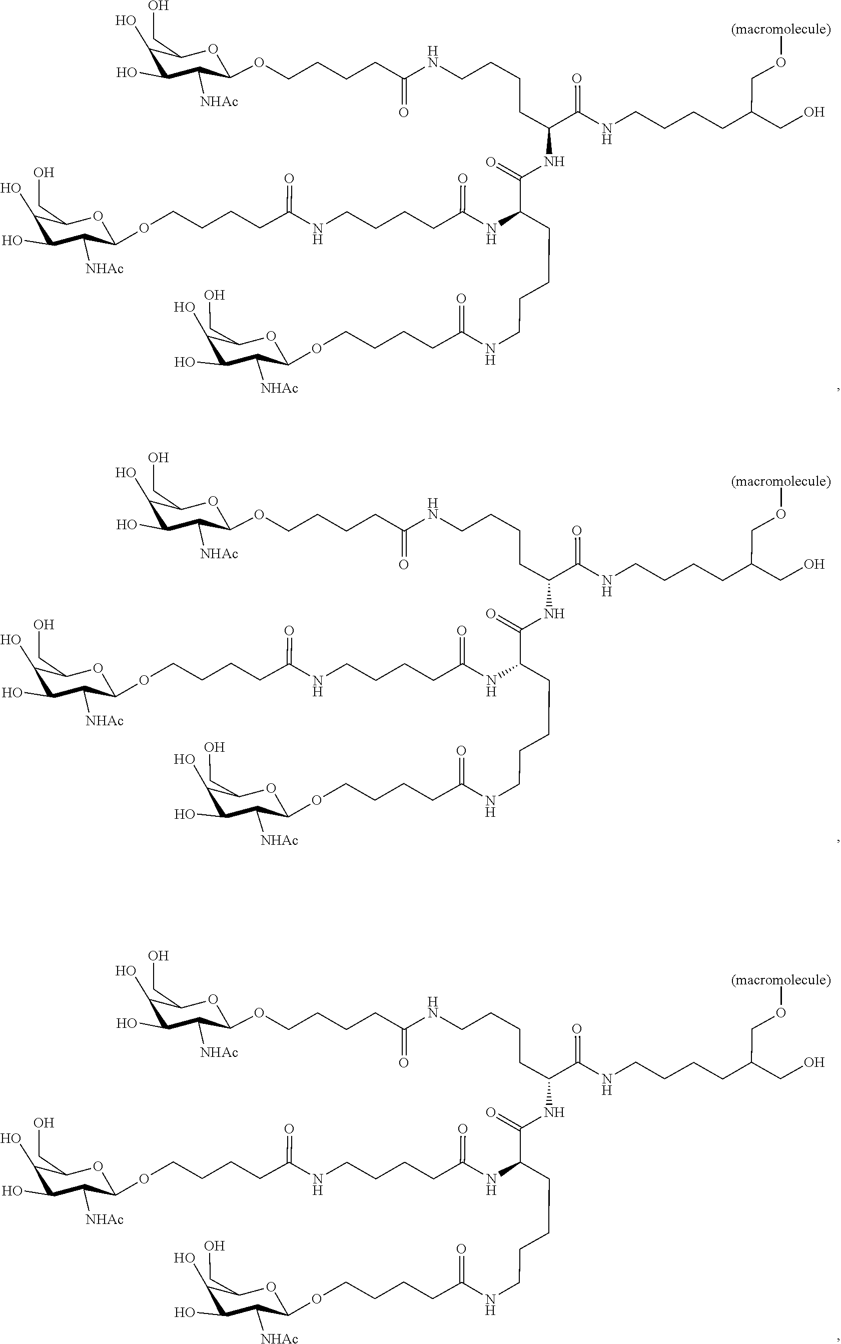

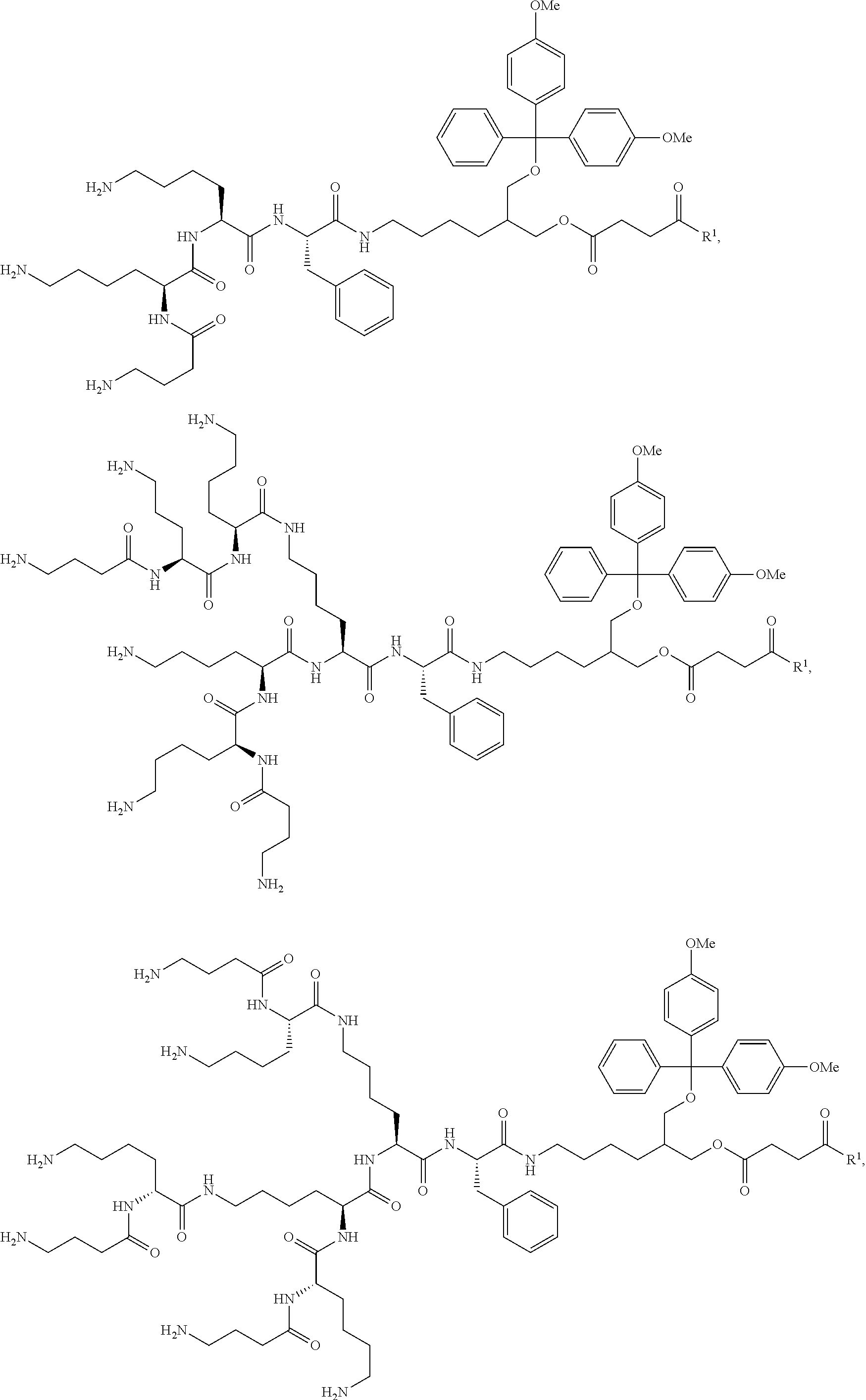

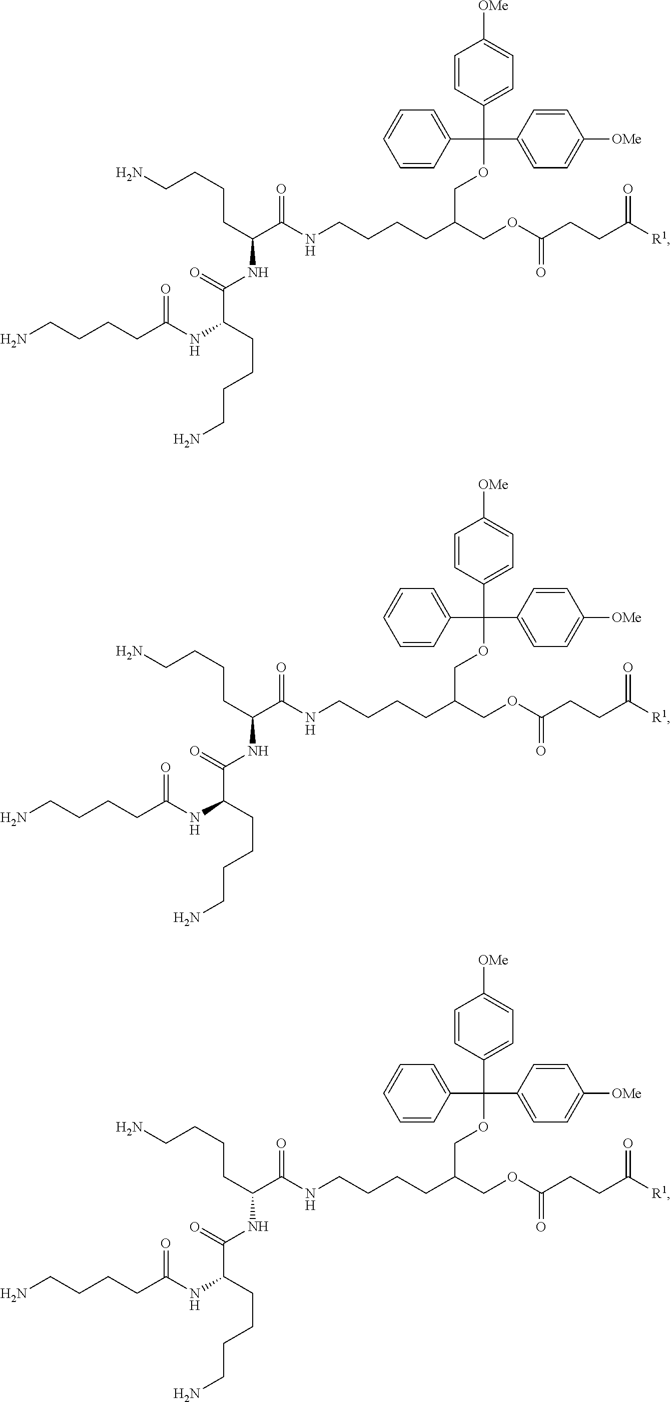

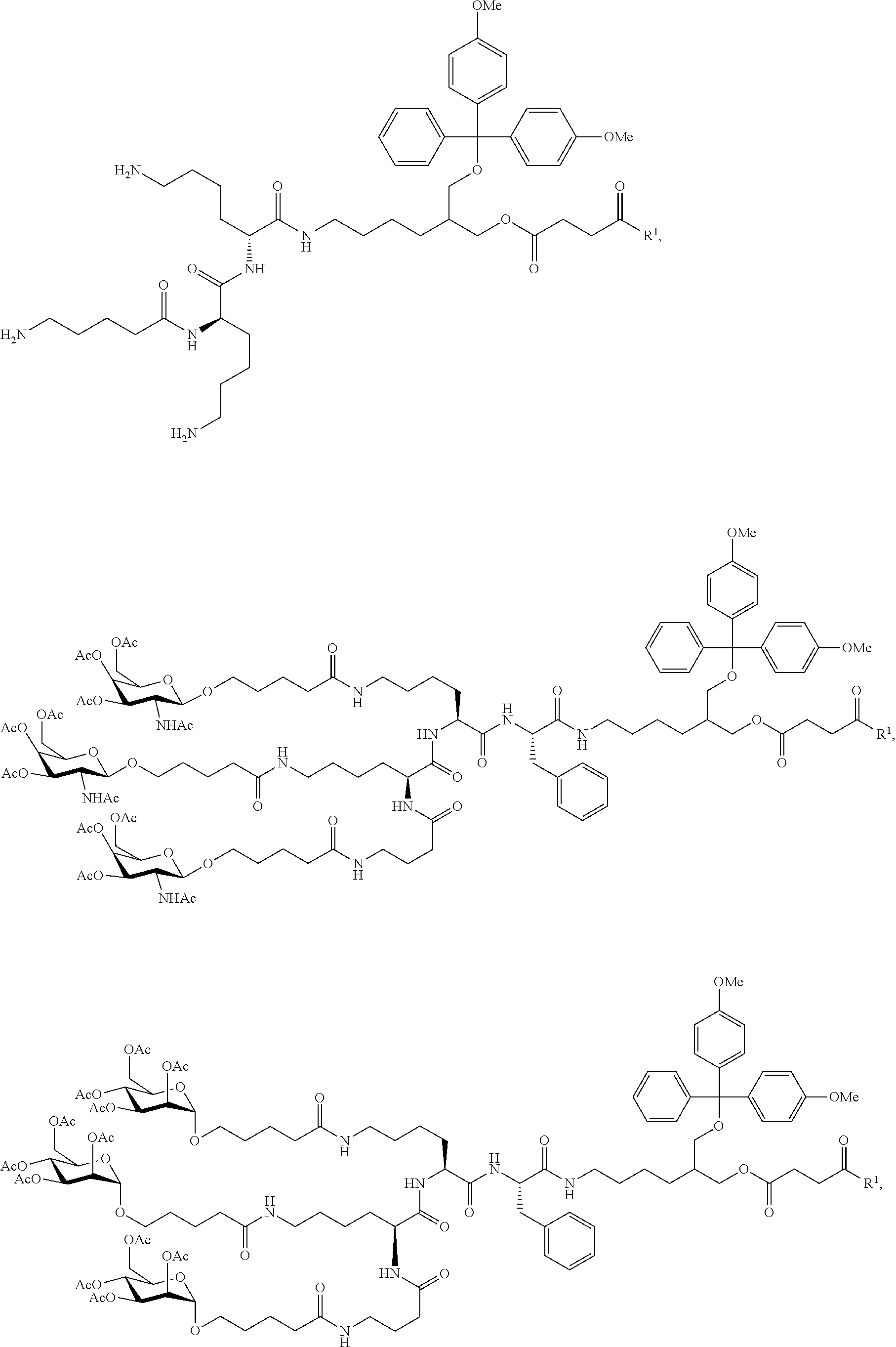

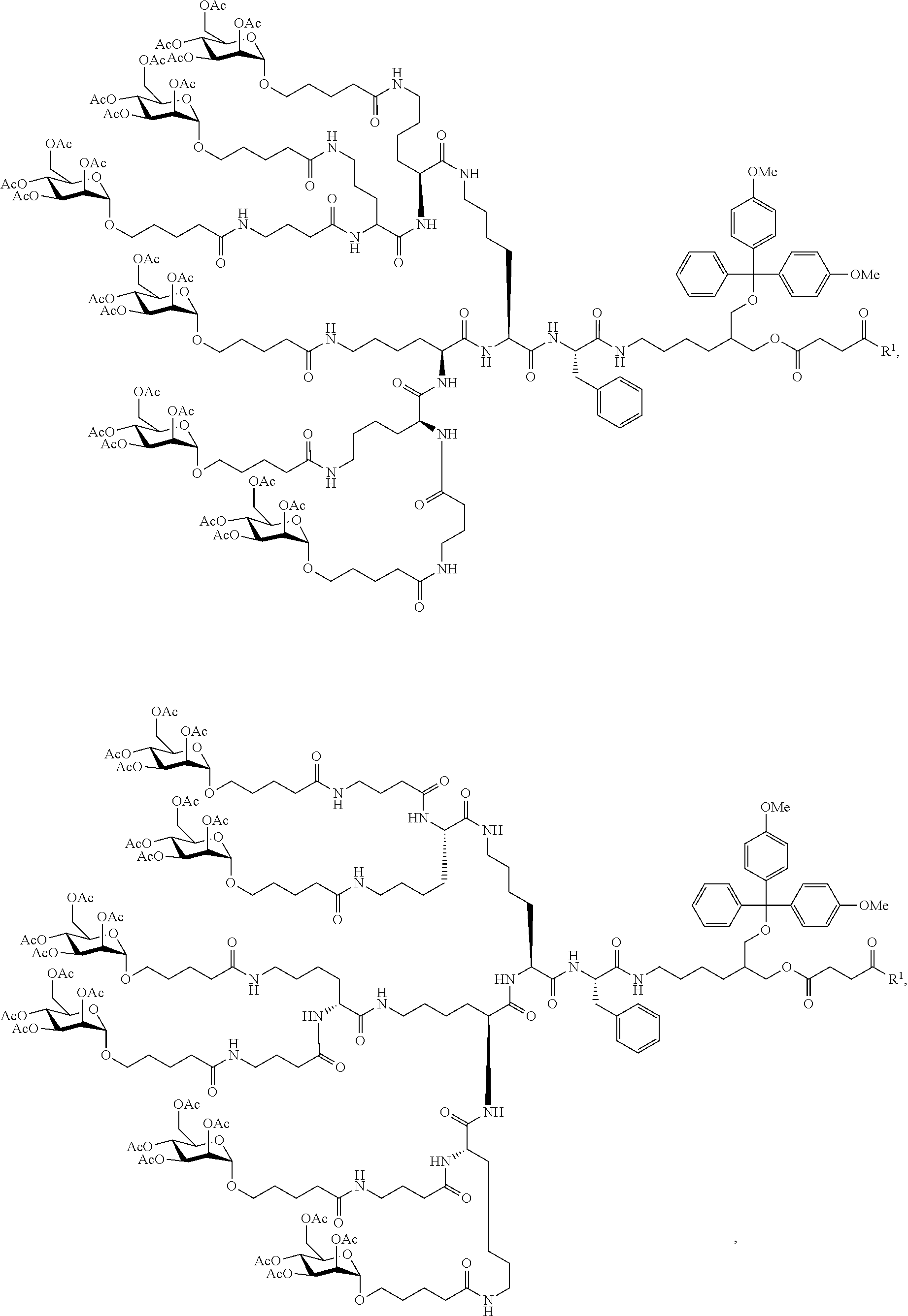

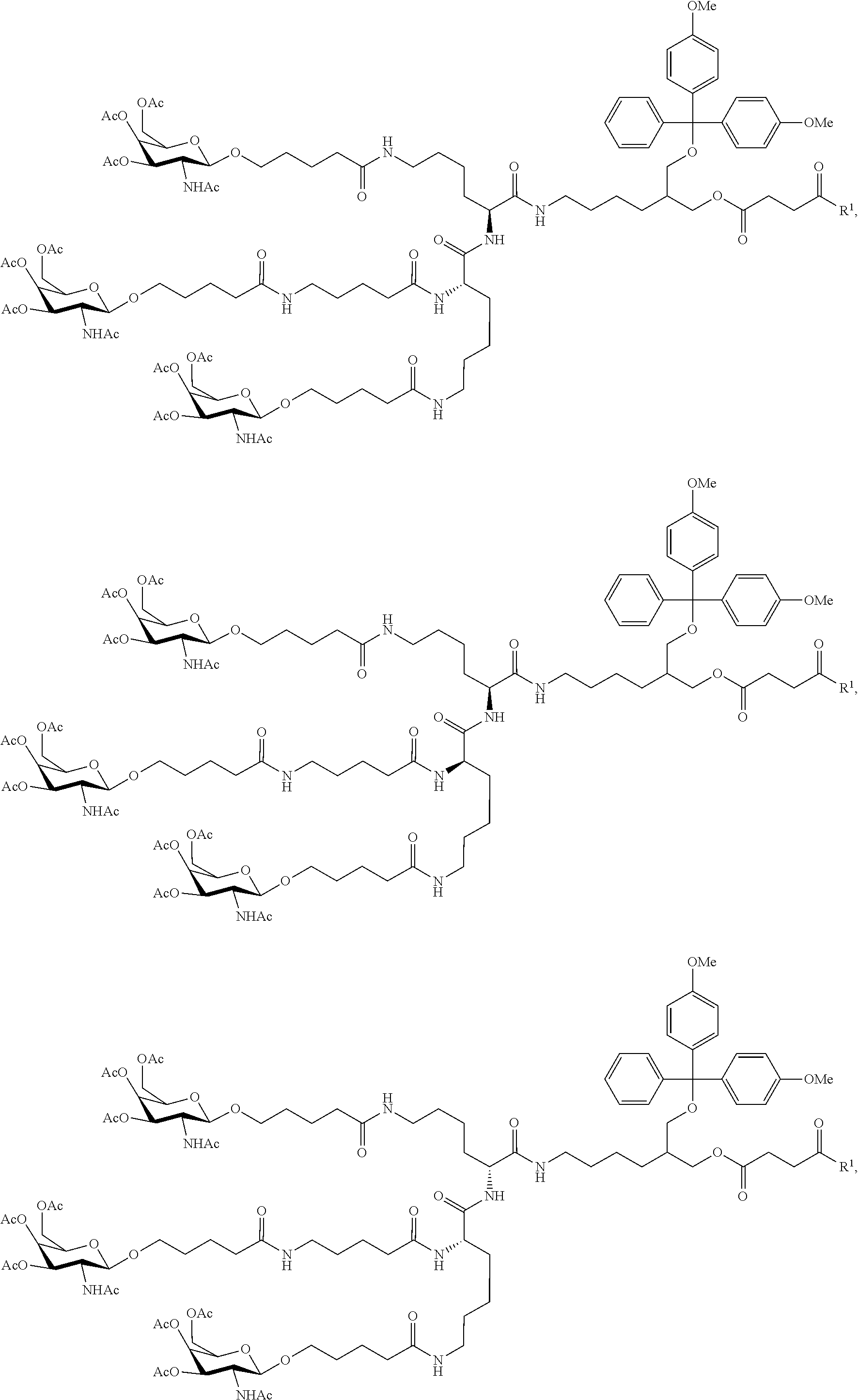

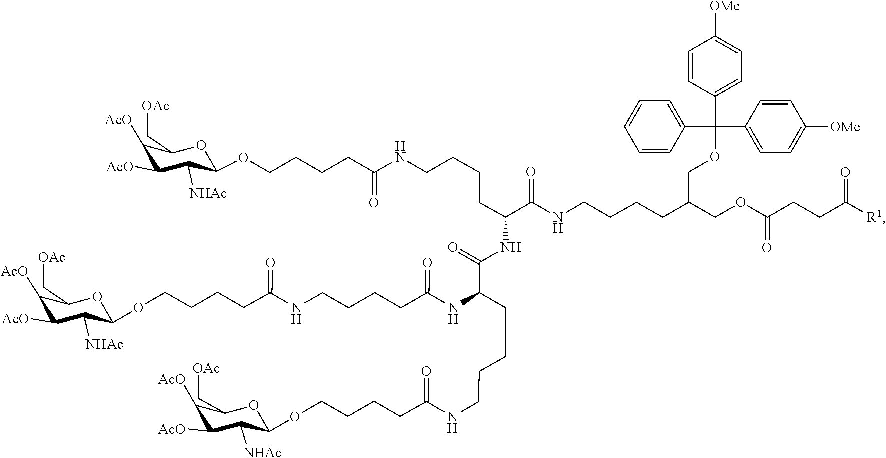

or a salt thereof,
wherein
R¹ is a solid support.

In some embodiments of the compounds provided herein, the compound comprises a macromolecule covalently linked to one or more, independently, of the following formulae:

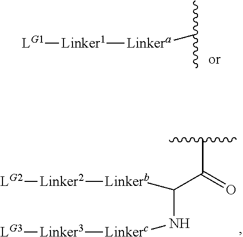

or a salt thereof,
wherein
L^{G1}, L^{G2}, and L^{G3} are each, independently, selected from a lipophilic group;
Linker¹, Linker², and Linker³ are each, independently, selected from a bond, C₁₋₂₀ alkyl, C₁₋₂₀ alkenyl, C₁₋₂₀ alkynyl, C₁₋₂₀ aralkyl, C₁₋₂₀ aralkenyl, C₁₋₂₀ aralkynyl, C₁₋₂₀ heteroaralkyl, C₁₋₂₀ heteroaralkenyl, C₁₋₂₀ heteroaralkynyl, —O—, —C(O)—, —NR—, —S—, —S(O)—, —SO₂—, —SO₂NH—, —NHSO₂—, —CₙH₂ₙ₊₂—, —CₙH₂ₙ—, —CₙH₂ₙ₋₂—, —S—S—, —RC═N—, —N═CR—, —O═N═C—, —C═N—O—, —O—C(O)—O—, —C(O)—NR—, —NR—C(O)—, —O—C(O)—NR—, —N R—C(O)—O—, —NR¹⁰—C(O)—NR²⁰—, —NR¹⁰—C(S)—NR²⁰—, or —NR¹⁰SO₂NR²⁰—;
Linker^{a}, Linker^{b}, and Linker^{c} are each, independently, selected from —O—, —NR—C(O)—, —C(O)—NR—, or —NR¹⁰—C(O)—NR²⁰—;
each n is, independently, more than 1; and
R, R¹⁰, and R²⁰ are each, independently, selected from H, C₁₋₂₀ alkyl, C₁₋₂₀ alkenyl, C₁₋₂₀ alkynyl, or an amino acid.

In some embodiments of the compounds provided herein, the compounds comprise a macromolecule covalently linked to one or more of the following formula:

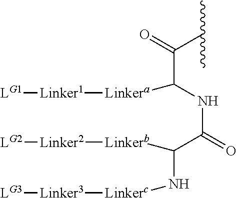

or a salt thereof.

In some embodiments of the compounds provided herein, the compounds comprise the following formula:

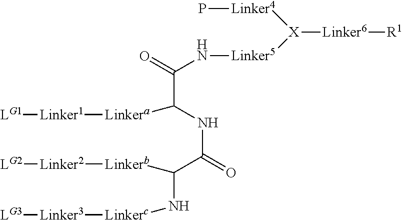

or a salt thereof,
wherein
Linker⁴, Linker⁵, and Linker⁶ are each, independently, selected from a bond, C₁₋₂₀ alkyl, C₁₋₂₀ alkenyl, C₁₋₂₀ alkynyl, C₁₋₂₀ aralkyl, C₁₋₂₀ aralkenyl, C₁₋₂₀ aralkynyl, C₁₋₂₀ heteroaralkyl, C₁₋₂₀ heteroaralkenyl, C₁₋₂₀ heteroaralkynyl, —O—, —C(O)—, —NR—, —S—, —S(O)—, —SO₂—, —SO₂NH—, —NHSO₂—, —CₙH₂ₙ₊₂—, —CₙH₂ₙ—, —CₙH₂ₙ₋₂—, —S—S—, —RC═N—, —N═CR—, —O═N═C—, —C═N—O—, —O—C(O)—O—, —C(O)—NR—, —NR—C(O)—, —O—C(O)—NR—, —N R—C(O)—O—, —NR¹⁰—C(O)—NR²⁰—, —NR¹⁰—C(S)—NR²⁰—, or —NR¹⁰SO₂NR²⁰—;
P is triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, trimethylsulfonyltriphenylmethyl;
X is CH or N; and
R¹ is a solid support.

In some embodiments of the compounds provided herein, the compounds comprise one or more, independently, of the following formula:

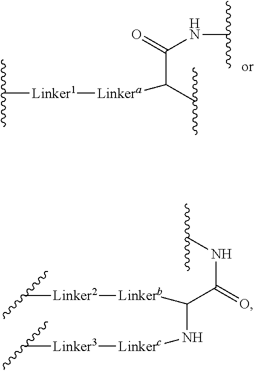

or a salt thereof,
wherein
Linker¹, Linker², and Linker³ are each, independently, selected from a bond, C₁₋₂₀ alkyl, C₁₋₂₀ alkenyl, C₁₋₂₀ alkynyl, C₁₋₂₀ aralkyl, C₁₋₂₀ aralkenyl, C₁₋₂₀ aralkynyl, C₁₋₂₀ heteroaralkyl, C₁₋₂₀ heteroaralkenyl, C₁₋₂₀ heteroaralkynyl, —O—, —C(O)—, —NR—, —S—, —S(O)—, —SO₂—, —SO₂NH—, —NHSO₂—, —CₙH₂ₙ₊₂—, —CₙH₂ₙ—, —CₙH₂ₙ₋₂—, —S—S—, —RC═N—, —N═CR—, —O═N═C—, —C═N—O—, —O—C(O)—O—, —C(O)—NR—, —NR—C(O)—, —O—C(O)—NR—, —N R—C(O)—O—, —NR¹⁰—C(O)—NR²⁰—, —NR¹⁰—C(S)—NR²⁰—, or —NR¹⁰SO₂NR²⁰—;
Linker^{a}, Linker^{b}, and Linker^{c} are each, independently, selected from —O—, —NR—C(O)—, —C(O)—NR—, or —NR¹⁰—C(O)—NR²⁰—;
each n is, independently, more than 1; and
R, R¹⁰, and R²⁰ are each, independently, selected from H, C₁₋₂₀ alkyl, C₁₋₂₀ alkenyl, C₁₋₂₀ alkynyl, or an amino acid.

In some embodiments of the compounds provided herein, the compounds comprise one or more of the following formulae:

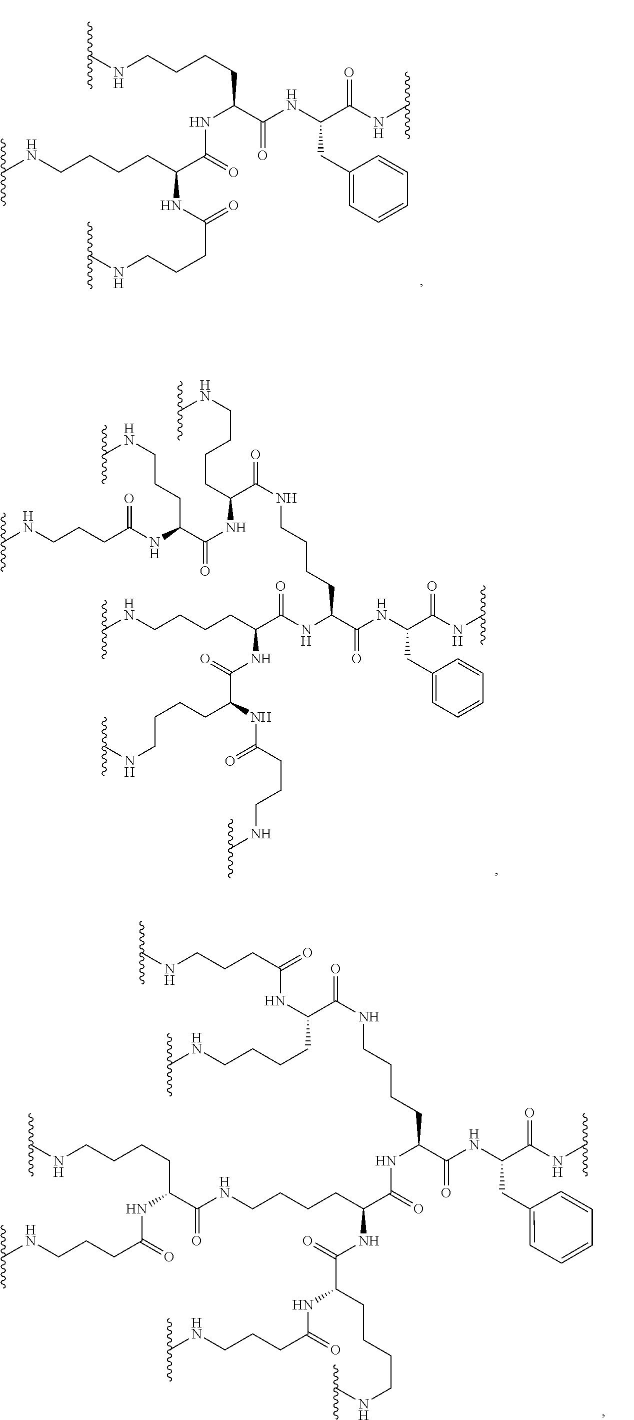 [SMILES: *NCCCC[C@H](NC(=O)CCCN*)C(=O)N[C@@H](CCCCN*)C(=O)N[C@@H](Cc1ccccc1)C(=O)N* ; *NCCCC[C@H](NC(=O)CCCN*)C(=O)N[C@@H](CCCCN*)C(=O)N[C@@H](CCCCNC(=O)[C@H](CCCCN*)NC(=O)[C@H](CCCN*)NC(=O)CCCN*)C(=O)N[C@@H](Cc1ccccc1)C(=O)N* ; *NCCCC[C@H](NC(=O)CCCN*)C(=O)NCCCC[C@H](NC(=O)[C@H](CCCCNC(=O)[C@@H](CCCCN*)NC(=O)CCCN*)NC(=O)[C@H](CCCCN*)NC(=O)CCCN*)C(=O)N[C@@H](Cc1ccccc1)C(=O)N*]

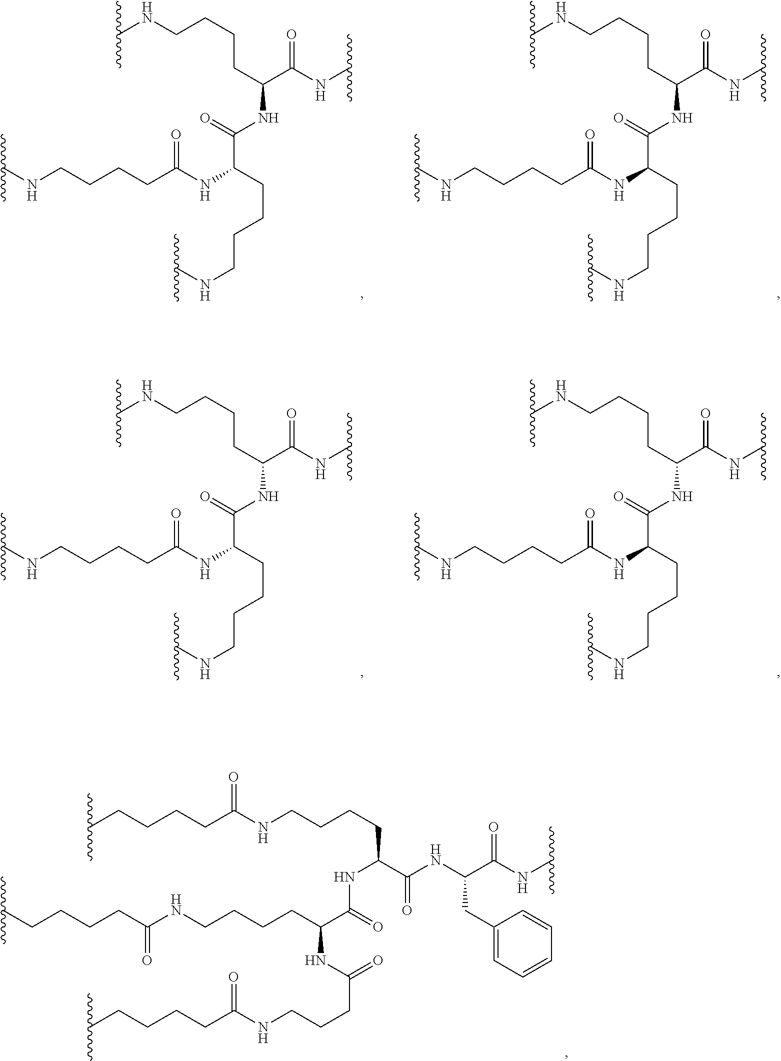 [SMILES: *NCCCCC(=O)N[C@@H](CCCCN*)C(=O)N[C@@H](CCCCN*)C(=O)N* ; *NCCCCC(=O)N[C@H](CCCCN*)C(=O)N[C@@H](CCCCN*)C(=O)N* ; *NCCCCC(=O)N[C@@H](CCCCN*)C(=O)N[C@H](CCCCN*)C(=O)N* ; *NCCCCC(=O)N[C@H](CCCCN*)C(=O)N[C@H](CCCCN*)C(=O)N* ; *CCCCC(=O)NCCCC[C@H](NC(=O)CCCNC(=O)CCCC*)C(=O)N[C@@H](CCCCNC(=O)CCCC*)C(=O)N[C@@H](Cc1ccccc1)C(=O)N*]

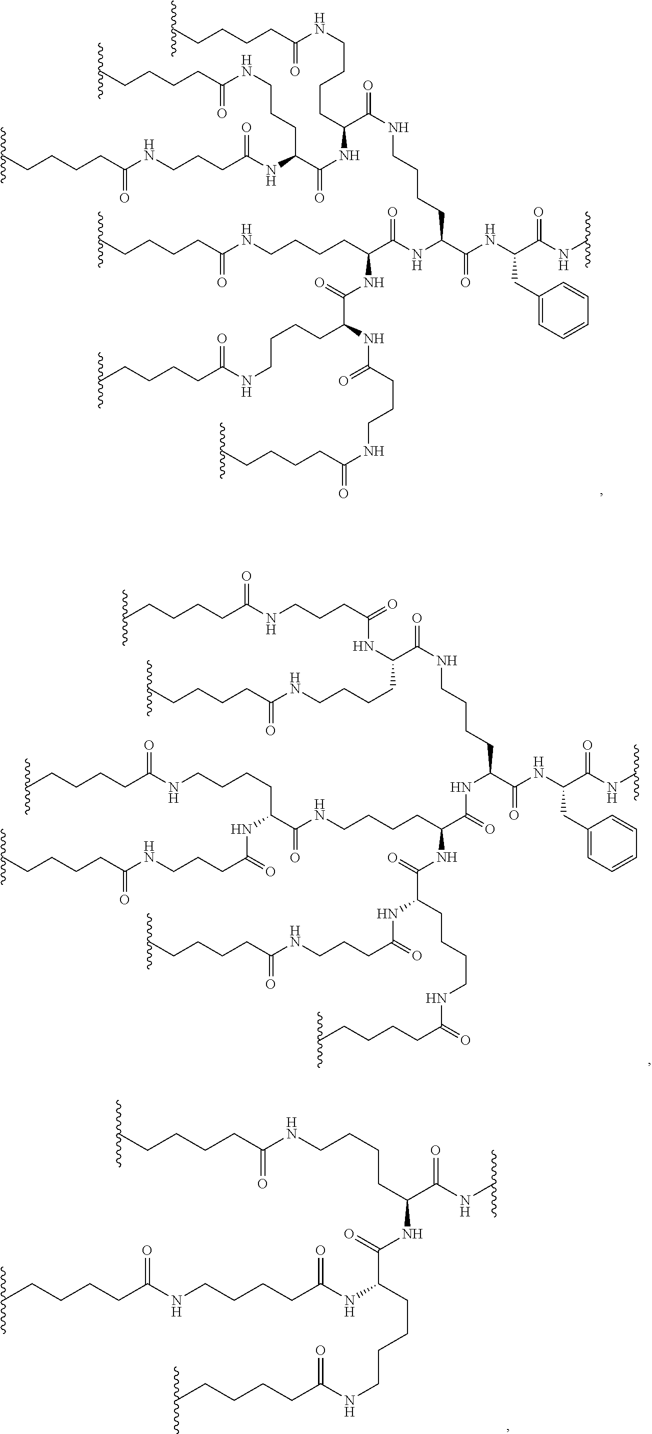 [SMILES: *CCCCC(=O)NCCCC[C@H](NC(=O)CCCNC(=O)CCCC*)C(=O)N[C@@H](CCCCNC(=O)CCCC*)C(=O)N[C@@H](CCCCNC(=O)[C@H](CCCCNC(=O)CCCC*)NC(=O)[C@H](CCCNC(=O)CCCC*)NC(=O)CCCNC(=O)CCCC*)C(=O)N[C@@H](Cc1ccccc1)C(=O)N* ; *CCCCC(=O)NCCCC[C@H](NC(=O)CCCNC(=O)CCCC*)C(=O)NCCCC[C@H](NC(=O)[C@H](CCCCNC(=O)[C@@H](CCCCNC(=O)CCCC*)NC(=O)CCCNC(=O)CCCC*)NC(=O)[C@H](CCCCNC(=O)CCCC*)NC(=O)CCCNC(=O)CCCC*)C(=O)N[C@@H](Cc1ccccc1)C(=O)N* ; *CCCCC(=O)NCCCCC(=O)N[C@@H](CCCCNC(=O)CCCC*)C(=O)N[C@@H](CCCCNC(=O)CCCC*)C(=O)N*]

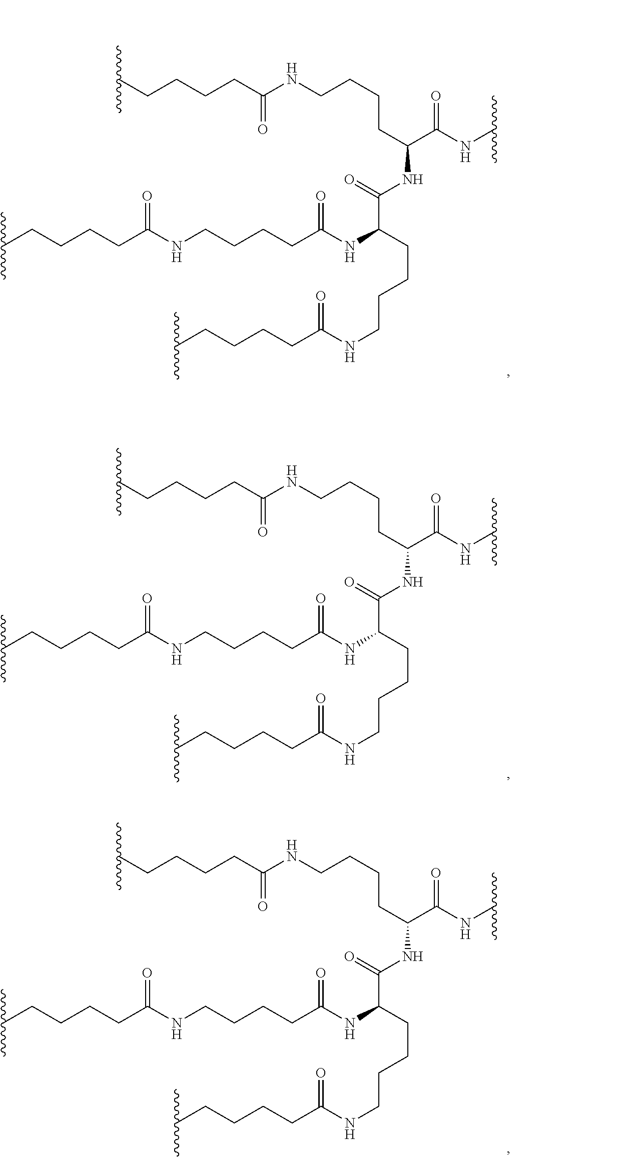 [SMILES: *CCCCC(=O)NCCCCC(=O)N[C@H](CCCCNC(=O)CCCC*)C(=O)N[C@@H](CCCCNC(=O)CCCC*)C(=O)N* ; *CCCCC(=O)NCCCCC(=O)N[C@@H](CCCCNC(=O)CCCC*)C(=O)N[C@H](CCCCNC(=O)CCCC*)C(=O)N* ; *CCCCC(=O)NCCCCC(=O)N[C@H](CCCCNC(=O)CCCC*)C(=O)N[C@H](CCCCNC(=O)CCCC*)C(=O)N*]

or a salt thereof.

In some embodiments, the compounds provided herein are in the form of a pharmaceutically acceptable salt. As used herein, the term “pharmaceutically acceptable salt” refers to derivatives of the compounds provided herein wherein the parent compound is modified by converting one or more of an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the compounds provided herein include the conventional non-toxic salts of the parent compound formed, for example, from non-toxic inorganic or organic acids. The pharmaceutically acceptable salts of the compounds provided herein can be synthesized from the parent compound which contains one or more basic or acidic moieties by conventional chemical methods. Generally, such salts can be prepared by combining the free acid or base forms of these compounds with a stoichiometric amount (relative to the number of moieties to be converted to a corresponding salt) of the appropriate base or acid in water or in an organic solvent, or in a mixture of the two; generally, nonaqueous media such as ether, ethyl acetate, ethanol, isopropanol, or acetonitrile may be used. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

Also provided herein are compositions, comprising the compounds or functionalized macromolecules provided herein. In some embodiments, the composition is a pharmaceutical composition further comprising one or more pharmaceutically acceptable excipients or carriers. As used herein, the term “excipient” or “carrier” refers to physiologically compatible additives useful in preparation of a pharmaceutical composition. Examples of pharmaceutically acceptable carriers and excipients can, for example, be found in Remington Pharmaceutical Science, 17th Ed.

In some embodiments, the compounds, functionalized macromolecules, or compositions provided herein are housed within a container, optionally wherein the container reduces or blocks transmission of visible or ultraviolet light through the container. In some embodiments, the compounds, housed within the container, undergo photolysis at a slower rate as compared to a container that does not reduce or block transmission of visible or ultraviolet light. In some embodiments, the compounds, when housed within the container, have a rate of photolysis that is about zero.

In some embodiments, provided herein are solid supports for the synthesis of macromolecules. In some embodiments, the supports comprise bioconjugation linkers, which may also be referred to herein as functional moieties or described by the formulae provided herein.

In some embodiments, the functional moieties referred to herein comprise one or more of the formulae provided herein. In some embodiments, where the formulae include a macromolecule or a solid support component, the functional moiety may be the portion of the formulae not including the macromolecule or solid support component. Thus, in some embodiments, provided herein are macromolecules comprising one or more functional moieties independently, selected from L^{J-1}, L^{J-2}, L^{J-3} L^{J-4}, L^{J-5}, L^{J-6}, L^{J-7}, L^{J-8}, L^{J-9}, or L^{J-10}, where the moieties are defined as in the Examples below.

In some embodiments of the present disclosure, the macromolecule referred to is a biopolymer, or a mixture of different biopolymers. In some embodiments, the macromolecule is a peptide, an oligonucleotide, an oligosaccharide, or a mixture thereof. In some embodiments, the macromolecule is an oligonucleotide having one or two functional moieties. In some embodiments, the oligonucleotide is about 10 to about 25 nucleotides in length. In some embodiments, the oligonucleotide is about 14 to about 21 nucleotides in length. In some embodiments, the oligonucleotide is about 14 to about 18 nucleotides in length. In some embodiments, the oligonucleotide is a DNA or RNA oligonucleotide. In some embodiments, the oligonucleotide is a modified DNA or RNA oligonucleotide, or a combination thereof. In some embodiments, the oligonucleotide comprises a mixture of modified and unmodified nucleotides. In some embodiments, the oligonucleotide is SEQ ID NO: 1 or SEQ ID NO:2, as defined in the Examples below. In some embodiments, the functional moiety is at the 5′-end of the oligonucleotide. In some embodiments, the functional moiety is at the 3′-end of the oligonucleotide. In some embodiments, a functional moiety is at the 5′-end of the oligonucleotide and another functional moiety is at the 3′-end of the oligonucleotide. Similarly, in some embodiments, the macromolecule is a peptide having one or two functional moieties. In some embodiments, the functional moiety is at the N-terminus of the peptide. In some embodiments, the functional moiety is at the C-terminus of the peptide. In some embodiments, a functional moiety is at the N-terminus of the peptide and another functional moiety is at the C-terminus of the peptide. In some embodiments, the functional moieties are selected, independently, from L^{J-1}, L^{J-2}, L^{J-3} L^{J-4}, L^{J-5}, L^{J-6}, L^{J-7}, L^{J-8}, L^{J-9}, or L^{J-10}, where the moieties are defined as in the Examples below.

In some embodiments, the macromolecule comprises SEQ ID NO: 1 or SEQ ID NO:2, as defined in the Examples below, and one or two functional moieties selected, independently, from L^{J-1}, L^{J-2}, L^{J-3}, L^{J-4}, L^{J-5}, L^{J-6}, L^{J-7}, L^{J-8}, L^{J-9}, or L^{J-10}, where the moieties are defined as in the Examples below.

In some embodiments, provided herein are synthetic processes of solid support platforms for the synthesis of oligonucleotides by automated oligonucleotide synthetic cycles and post-synthetic procedures. In some embodiments, provided herein are processes for facile functionalization of oligonucleotides by changing linkers and ligands.

In some embodiments, the solid support provided herein includes one or more of: solid support (i.e. R¹); bridge X; modifier P; functional ligands L^{G1}-L^{G3}; and a multivalent peptidyl backbone wherein each component is connected by Linker⁵. In some embodiments, functional ligands L^{G1}-L^{G3} are, independently, connected to the multivalent peptidyl backbone by two flexible linkers selected from Linker¹-Linker³ and Linker^{a}-Linker^{c}, where L^{G1} is through Linker¹-Linker^{a}, L^{G2} is through Linker²-Linker^{b}, and L^{G3} is through Linker³-Linker^{c}. Bridge X is a branching point to connect modifier P through Linker⁴, the multivalent peptidyl backbone through Linker⁵, and the solid support through Linker⁶.

In some embodiments, the solid supports provided herein may be prepared by solid phase peptide synthesis. In some embodiments, the solid supports provided herein may be synthesized by peptidyl bond formation and functionalization of linkers and ligands from solid support, or by coupling the functionalized ligand-containing molecule(s) to a solid support.

In some embodiments, the solid supports provided herein are used for the synthesis of functionalized oligonucleotides, functionalized peptides, or functionalized oligosaccharides. Modifier P is removed, in some embodiments, to give the starting point of solid phase oligonucleotide synthesis, for example. Synthesis and purification processes of functionalized macromolecules may follow the standard procedure of solid phase oligonucleotide synthesis, or solid phase peptide synthesis, or a combination of both. In some embodiments, standard nucleic acids may be synthesized, including DNA and RNA. In some embodiments, non-standard nucleic acids may be synthesized. In some embodiments, the non-standard nucleic acids may be a threose nucleic acid (TNA), a hexose or hexitol nucleic acid (HNA), a xeno-nucleic acid (XNA), a locked-nucleic acid (LNA), a formyl glycerol nucleic acid (FNA), a glycerol nucleic acid (GNA), or a peptide nucleic acid, or a combination thereof. In some embodiments, the nucleic acids may be substituted by one or more of a 3′- or 2′-O-Me or a 3′- or 2′-fluoro or a combination thereof. In some embodiments, the nucleic acids comprise a combination of non-standard and standard nucleic acid moieties. In some embodiments, the nucleic acids comprise one or more C-nucleosides, where the glycosidic bond of a given nucleotide is a carbon-carbon bond.

In some embodiments, the solid support is selected from, but not limited to, silica gel, controlled pore glass (CPG), or polystyrene resin (PS).

In some embodiments, L^{G1}, L^{G2}, and L^{G3} are functionalized ligands independently selected from lipophilic groups including, but not limited to, small molecule (such as biotin, fluorescent dyes including, but not limited to, Cy3 or Cy5), proteins, antibody (such as brentuximabvedotin and gemtuzumab ozogamicin), oligosaccharide, nucleic acids, synthetic polymers, carbohydrates, and lipids, which may be the same or different from each other.

In some embodiments, carbohydrates include, but are not limited to, monosaccharide, disaccharides, trisaccharides, tetrasaccharides, polysaccharides or their modified derivatives.

In some embodiments, lipids include, but are not limited to, fatty acids, glycerides, sterols, and their modified derivatives.

In some embodiments, Linker¹⁻⁶ and Linker^{a-c} are linkers connecting the various functional components of a solid support platform.

In some embodiments, Linker¹⁻⁶ are independently selected from, but not limited to, a combination of a bond or alkyl, (straight alkyl, branched alkyl, or cycloalkyl), alkenyl, and alkynyl chains with C₁-C₂₀ containing aralkyl, aralkenyl, aralkynyl, heteroaralkyl, heteroaralkenyl, heteroarakynyl, —O—, —C(O)—, —NR—, —S—, —S(O)—, —SO₂—, —SO₂NH—, —NHSO₂—, —CₙH₂ₙ₊₂—, —CₙH₂ₙ—, —CH₂ₙ₋₂—, where n is more than 1, —S—S—, —RC═N—, —N═CR—, —O═N═C—, —C═N—O—, —O—C(O)—O—, —C(O)—NR—, —NR—C(O)—, —O—C(O)—NR—, —N R—C(O)—O—, —NR¹⁰—C(O)—NR²⁰—, —NR¹⁰—C(S)—NR²⁰—, —NR¹⁰SO₂NR²⁰— where R, R¹⁰ and R²⁰ is selected from hydrogen, straight and/or branched alkyl, alkenyl and alkynyl chains with C₁-C₂₀, natural amino acids, and modified amino acids, which may be the same or different from each other.

In some embodiments, Linker^{a-c} are linkers connecting Linker¹⁻³ to multivalent peptidyl backbone, and independently selected from, but not limited to, —O—, —NR—C(O)—, —C(O)—NR—, —NR¹⁰—C(O)—NR²⁰—, where R, R¹⁰ and R²⁰ is selected from hydrogen, straight and/or branched alkyl, alkenyl and alkynyl chains with C₁-C₂₀, natural amino acids, and modified amino acids, which may be the same or different from each other.

In some embodiments, Linker⁴ is used as a linker to connect the bridge X to modifier P, Linker⁵ is the linker to connect the bridge X to multivalent peptidyl backbone, and Linker⁶ is the linker to connect the bridge X to the solid support.

In some embodiments, X is the bridge to connect the three functionalized components solid support, modifier, and multivalent peptidyl backbone. In some embodiments, X is selected from, but not limited to, carbon as CH and nitrogen as N.

In some embodiments, modifier P is a starting point for the synthesis of a functionalized compound or macromolecule, and is selected from, but not limited to, an acid-labile moiety. In some embodiments, the acid-labile moiety is selected from triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl.

In some embodiments, multivalent peptidyl backbone is composed of at least two peptide bonds where multivalency is from two side chains and backbone amine of two amino acids selected from natural amino acids and modified amino acids containing functionalized side chains such as alcohol, phenol, thiol, amine, carboxylic acid, and amide.

In some embodiments, provided herein are solid supports including one or more of the formulae described herein, which in some embodiments are precursors and solid support platforms used for the synthesis of functionalized macromolecules, which in some embodiments are oligonucleotides.

In some embodiments of the formulae provided herein, the solid support is silica gel, controlled pore glass (CPG), or polystyrene resin (PS), and L^{G1}, L^{G2}, and L^{G3} are null (i.e. H) or galactosamine tetraacetate.

In some embodiments of the formulae provided herein, the solid support is controlled pore glass (CPG) or polystyrene resin (PS), and L^{G1}, L^{G2}, and L^{G3} are null (i.e. H) or galactosamine tetraacetate.

In some embodiments of the formulae provided herein, the solid support is CPG or PS, and L^{G1}, L^{G2}, and L^{G3} are null (i.e. H) or mannose tetraacetate.

In some embodiments of the formulae provided herein, the solid support is CPG or PS, L^{G1} is null (i.e. H) or three of mannose tetraacetates connected multivalent peptidyl backbone through modified di-lysine Linker, L^{G2} is composed of one mannose tetraacetates connected to side chain of multivalent peptidyl backbone, and L G3 is composed two mannose tetracetates connected to multivalent peptidyl backbone through modified lysine Linker.

In some embodiments of the formulae provided herein, the solid support is CPG or PS, and L^{G1}, L^{G2} and L^{G3} are null (i.e. H) or two of mannose tetraacetates connected to multivalent peptidyl backbone through modified lysine linker for each.

In some embodiments, Linker¹⁻³ is C₅ carbonyl derivatives linking the sugars and Linker^{a-c}.

In some embodiments, Linker^{a-c} is side chain of lysine or gamma-butyric acid linking the Linker¹⁻³ and multivalent peptidyl backbone.

In some embodiments, Linker⁴ is C₁ ether linkage to connect the bridge X and Modifier P.

In some embodiments, Linker⁵ is composed of phenylalanine and straight C₄ aminoalkyl chain.

In some embodiments, Linker⁶ is C₁ ester linking the bridge X to succinate-bound solid support.

In some embodiments, Linker⁵ is composed of phenylalanine and straight C₄ aminoalkyl chain, and Linker⁶ is C₁ ester linking the bridge X to succinate-bound solid support.

In some embodiments, bridge X is CH, and modifier P is 4,4′-dimethoxytriphenylmethyl. In some embodiments, bridge X is N and Modifier P is 4,4′-dimethoxytriphenylmethyl.

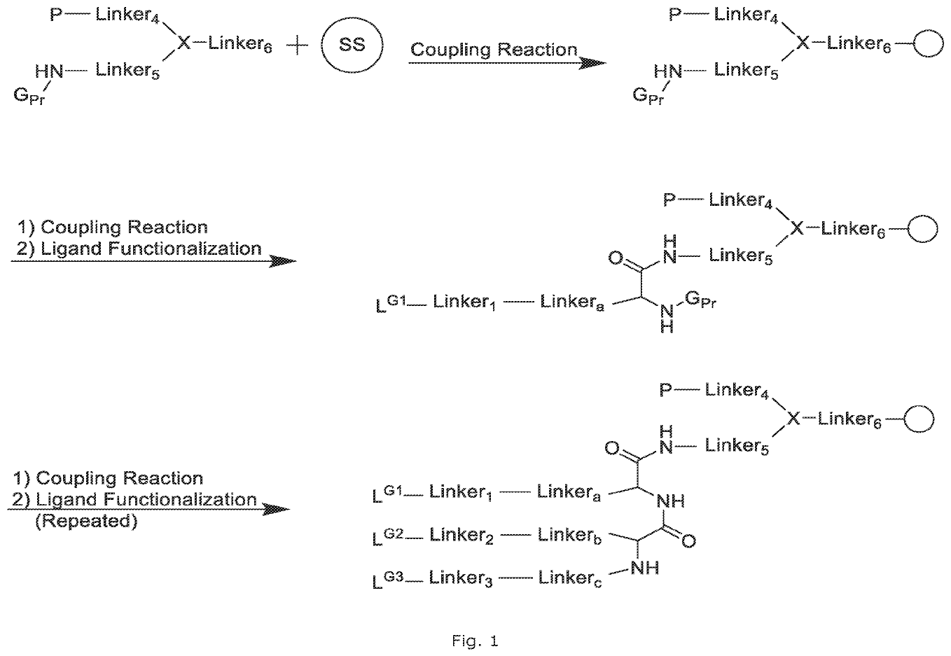

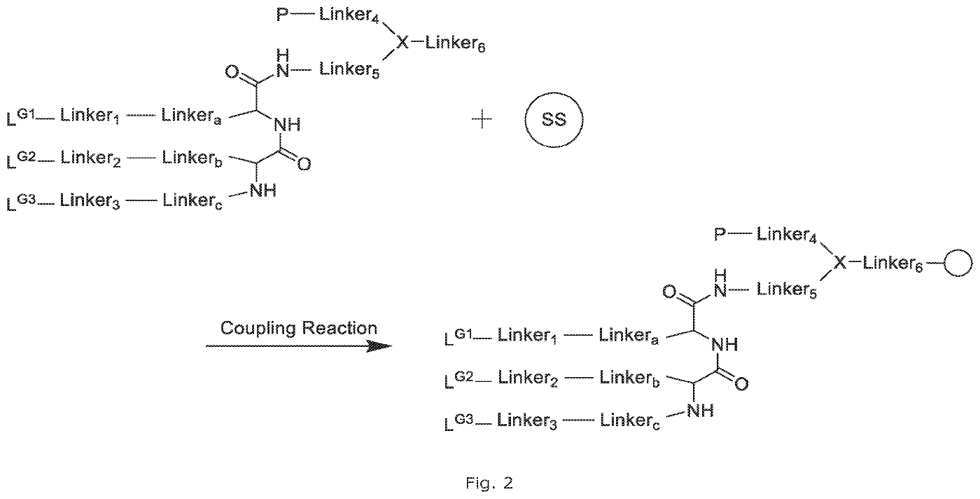

In some embodiments, provided herein are synthetic processes for the solid support platform according to the formulae described herein. In some embodiments, the solid support platforms are synthesized by peptidyl bond formation and functionalization of linkers and ligands, starting from a solid support. In some embodiments, the solid support is connected to Linker⁶ containing modifier P connected to Linker⁴ and amino functional group connected to Linker⁵. In some embodiments, a multivalent peptidyl backbone is step-by-step elongated by solid phase peptide synthesis and their side chain is modified to the functionalized ligands L^{G1}-L^{G3} by Linker¹⁻³ and Linker^{a-c} as shown in FIG. 1. In some embodiments, the solid support platforms are synthesized by coupling the functionalized ligands containing molecule to a solid support as shown in FIG. 2. In some embodiments, the functionalized ligands containing molecule is synthesized by solid phase peptide synthesis or solution phase peptide synthesis. In some embodiments, the solid support platforms possess the same structure and functionality regardless of synthetic routes. That is to say, in some embodiments, the solid support, to which the formulae provided herein may be covalently linked, is stable to the chemical conditions of solid support synthesis.

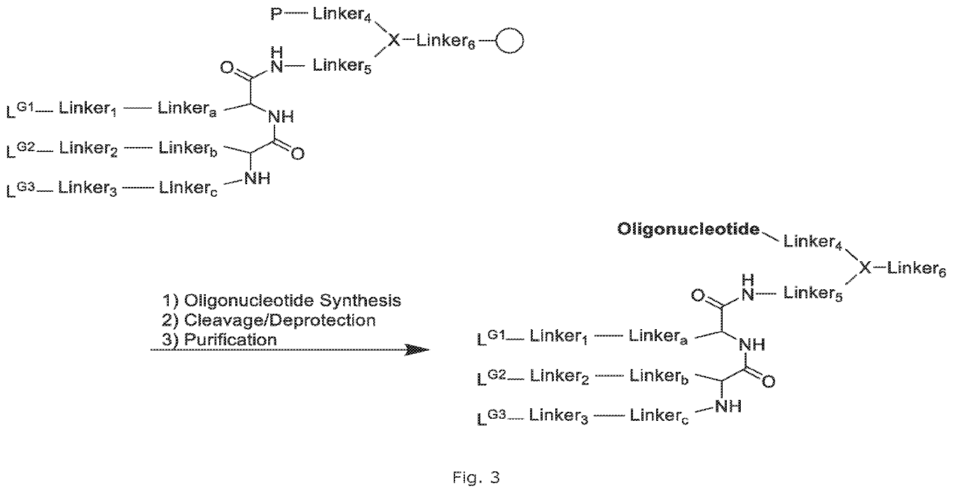

In some embodiments, provided herein are synthetic processes using solid support platform for the synthesis of oligonucleotides. In some embodiments, the oligonucleotide is functionalized with a formulae provided herein. In some embodiments, the solid support platform includes one of the formulae provided herein. In some embodiments, the solid support platforms containing modifier P are deblocked by trityl deprotection to give the starting point of solid phase oligonucleotide synthesis and oligonucleotide is synthesized through automated oligonucleotide synthetic cycles comprising chemical steps of one or more of deblocking, coupling, capping, and oxidation. In some embodiments, an oligonucleotide so prepared is processed by post-synthesis procedures comprising one or more of solid support cleavage, deprotection, purification, and characterization as shown in FIG. 3.

In some embodiments, the solid support may be any material that is stable to the chemical conditions of the particular synthetic cycle being used for solid support synthesis. In some embodiments, the solid support is a silica based or polymer based support. In some embodiments, the solid support is selected from, polymer-bound supports. 2-chlorotrityl resins. PEG resins, polystyrene resins. TentaGel resin, trityl resins, wang resins, carboxypolystyrene resins. Chelex sodium form resins, gum rosin natural resin, HypoGel resins, iodopolystyrene resins. 4-methylpolystyrene resisn, micro particles based on melamine resin. PAM resin, PEGA resin, Phoxime resin. 2-pyridine-co-Merrifield resin. REM resin. Rink amide (aminomethyl) polystyrene resin, hybrid resin, silica gel or controlled pore glass (CPG), or a combination thereof. In some embodiments, and in contrast to organic solid phase synthesis and peptide synthesis, the synthesis of oligonucleotides proceeds on non-swellable or low-swellable solid supports, which may result in an otherwise higher overall yield or purity, or both. In some embodiments, the solid supports are silica gel, controlled pore glass (CPG), and polystyrene resins. In some embodiments, the solid support comprises silica gel, controlled pore glass (CPG), polystyrene resin (PS), or a combination thereof. In some embodiments, the solid support comprises silica gel covalently linked with polystyrene. In some embodiments, the solid supports are controlled pore glass (CPG) and polystyrene resins. In some embodiments, the CPG is defined by its pore size. In some embodiments, including for oligonucleotide synthesis, pore sizes of 500, 1000, 1500, 2000 and 3000 Å are used to allow the preparation of various length of oligonucleotides. In some embodiments, the solid support CPG used may be further extended through aminoalkyl linker to result in long chain aminoalkyl (LCAA) CPG, which may be more suitable for synthesis of oligonucleotides over about 40 nucleotides in sequence length. In some embodiments, the oligonucleotide sequence length is about 50 or more, about 75 or more, about 100 or more, or about 150 or more nucleotides in length. In some embodiments, the solid support is a polystyrene resin. In some embodiments, polystyrene resin is suitable for oligonucleotide synthesis having a low-swellable, highly cross-linked polystyrene which contains an aminomethyl Linker. In some embodiments, the solid support is CPG or polystyrene.

In some embodiments, the building blocks referred to herein include solid support, bridge X, modifier P, functional ligands L^{G1}-L^{G3} and multivalent peptidyl backbone wherein each component is connected by Linkers. In some embodiments, functional ligands L^{G1}-L^{G3} are connected to the multivalent peptidyl backbone by two of flexible linkers selected from Linker¹⁻³ and Linker^{a-c}, where L^{G1} is through Linker¹-Linker^{a}, L^{G2} is through Linker²-Linker^{b}, and L^{G3} is through Linker³-Linker^{c}. In some embodiments, bridge X is a branching point to connect the modifier P through Linker⁴, the multivalent peptidyl backbone through Linker⁵, and the solid support through Linker⁶.

Solid support platform synthesis has several advantages over traditional macromolecule functionalization. Some advantages are that solid support platforms provide a method to diversify the functional bioconjugation ligands on demand. The number and species of bioconjugation linkers can be selected by changing the multivalent peptidyl backbone. Linkers, and functional ligands. For example, three functional ligands can be attached to trivalent peptidyl backbone. Six functional ligands can be added to hexavalent peptidyl backbone. The length of those functional ligands can extend or shrink as needed. Additionally, it is also possible to a heterogeneous mixture of bioconjugation ligands utilizing the step-by-step peptidyl backbone formation and ligand attachments.

Additional advantages are that solid support platforms provide a method to simplify the synthesis of functionalized oligonucleotides. For the synthesis of C3′ modified oligonucleotides, the synthetic process is usually composed of two stages: C3′ modification and oligonucleotide synthesis on solid phase, assuming the C3′ modification is well performed. However, in many cases. C3′ modification should be carefully monitored at every single modification stage. Otherwise, the functionalized oligonucleotides would be produced in low yield and/or contaminated with unwanted side products. Solid support platform is prepared in high purity and high yield in controlled synthetic process and in process control and provides the solid support suitable for the synthesis of functionalized oligonucleotides. Furthermore, solid support platform makes the synthetic process simplified affording high purity and high yield.

Those advantages of solid support platform are very important in the area of pharmaceutical application because the functionalized oligonucleotides should be provided in high purity and high yield for biological examinations, clinical tests, and commercialization.

In some embodiments of the formulae provided herein, bridge X is the junction point to connect the three functionalized components solid support, modifier P and multivalent peptidyl backbone. In some embodiments, bridge X is more than trivalent atom or functional group. In some embodiments, bridge X is selected from, but not limited to, carbon as trivalent CH, nitrogen as trivalent N, silicon as trivalent SiH, phosphous as trivalent P or trivalent P(O), trivalent cycloalkyl, trivalent cycloakenyl, trivalent cycloalkynyl, trivalent heterocycloalkyl, trivalent heterocycloalkenyl, trivalent heterocycloakyl, trivalent aralkyl, trivalent aralkenyl, trivalent aralkynyl, trivalent heteroaralkyl, trivalent heteroaralkenyl, trivalent heteroaralkynyl, trivalent aromatic, trivalent heteroaromatic or their modified derivatives. In some embodiments, bridge X is carbon as trivalent CH.

In some embodiments, modifier P is removed to give the starting point of solid phase oligonucleotide synthesis. In some embodiments, modifier P is selected from, but not limited to, pH labile functional groups. In some embodiments, removal of modifier P restores an unmodified terminal functional group, from which the oligonucleotide is synthesized through automated oligonucleotide synthetic cycles comprising deblocking, coupling, capping, and oxidation. In some embodiments, when the multivalent peptidyl backbone is synthesized by utilizing base labile functional groups, modifier P is chosen from acid labile functional groups and vice versa. In some embodiments, modifier P is also affected by the Linker⁴. Thus, in some embodiments, depending on the terminal functional group of the Linker⁴, modifier P is chosen from TCEP-labile or pH labile functional groups, considering the functional groups from multivalent peptidyl backbone synthesis. In some embodiments, modifier P is selected from, but not limited to, acid labile functional groups. In some embodiments, acid labile functional groups are selected from, but not limited to, the general formula —O—, —NH—, —C═NH—, —C(O)O—, and —OC(O)—. In some embodiments, acid labile functional groups are selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl if it is connected to the terminal alcohol through an ether linkage. In some embodiments, base labile functional groups are selected from, but not limited to, the general formula-O—, —NH—, —C═NH—, —C(O)O—, and —OC(O)—. In some embodiments, base or amine labile functional groups are selected from, but not limited to, fluorenylmethoxycarbonyl (Fmoc), 1-(4,4-dimethyl-2,6-dioxocyclohex-1-ylidene)ethyl (DDe), 1-(4,4-dimethyl-2,6-dioxocyclohex-1-ylidene) isovaleryl (ivDde), levulinate (Lev), or trifluoroacetate. In some embodiments, the group is 4,4′-dimethoxytriphenylmethyl connected to the terminal alcohol of Linker⁴.

In some embodiments, Linker⁴ is the linker to connect bridge X to modifier P, and provides a terminal functional group for automated solid phase oligonucleotide synthesis after removal of pH-labile functional group of modifier P. In some embodiments, Linker⁴ is selected from, but not limited to, combination of a bond or straight and/or branched alkyl, alkenyl and alkynyl chains with C₁-C₂₀ containing aralkyl, aralkenyl, aralkynyl, heteroaralkyl, heteroaralkenyl, heteroarakynyl, —O—, —C(O)—, —NR—, —S—, —S(O)—, —SO₂—, —SO₂NH—, —NHSO₂—, —CₙH₂ₙ₊₂—, —CₙH₂ₙ—, —CₙH₂ₙ₋₂—, where n is more than 1, —S—S—, —RC═N—, —N═CR—, —O═N═C—, —C═N—O—, —O—C(O)—O—, —C(O)—NR—, —NR—C(O)—, —O—C(O)—NR—, —N R—C(O)—O—, —NR¹⁰—C(O)—NR²⁰—, —NR¹⁰—C(S)—NR²⁰)—, —NR¹⁰SO₂NR²⁰)— where R, R¹⁰ and R²⁰ is selected from hydrogen, straight and/or branched alkyl, alkenyl and alkynyl chains with C₁-C₂₀, natural amino acids, modified amino acids, which may be the same or different from each other. In some embodiments, Linker⁴ is selected from straight alkyl chain with a bond or C₁-C₂₀ containing —O— and —NR— where R is selected from hydrogen. In some embodiments, Linker⁴ is selected from —CH₂—O—, which comprises alkyl chain with C₁ containing —O—.

In some embodiments, Linker⁵ is the linker to connect bridge X to multivalent peptidyl backbone. In some embodiments, Linker⁵ is selected from, but not limited to, combination of a bond or straight and/or branched alkyl, alkenyl and alkynyl chains with C₁-C₂₀ containing aralkyl, aralkenyl, aralkynyl, heteroaralkyl, heteroaralkenyl, heteroarakynyl, —O—, —C(O)—, —NR—, —S—, —S(O)—, —SO₂—, —SO₂NH—, —NHSO₂—, —CₙH₂ₙ₊₂—, —CH₂ₙ—, —CₙH₂ₙ₋₂—, where n is more than 1, —S—S—, —RC═N—, —N═CR—, —O—N—C—, —C═N—O—, —O—C(O)—O—, —C(O)—NR—, —NR—C(O)—, —O—C(O)—NR—, —N R—C(O)—O—, —NR¹⁰—C(O)—NR²⁰—, —NR¹⁰—C(S)—NR²⁰—, —NR¹⁰SO₂NR²⁰— where R, R¹⁰ and R²⁰ is selected from hydrogen, straight and/or branched alkyl, alkenyl and alkynyl chains with C₁-C₂₀, natural amino acids, modified amino acids, which may be the same or different from each other. In some embodiments, Linker⁵ is selected from a bond or straight chain C₁-C₂₀ alkyl. In some embodiments, Linker⁵ is a combination of straight alkyl chain with C₄ (saturated butyl) and phenylalanine connected to multivalent peptidyl backbone through peptide bond.

In some embodiments, Linker⁶ is the linker to connect bridge X to a solid support (such as R¹). In some embodiments, Linker⁶ is selected from, but not limited to, combination of a bond or straight and/or branched alkyl, alkenyl and alkynyl chains with C₁-C₂₀ containing aralkyl, aralkenyl, aralkynyl, heteroaralkyl, heteroaralkenyl, heteroarakynyl, —O—, —C(O)—, —NR—, —S—, —S(O)—, —SO₂—, —SO₂NH—, —NHSO₂—, —CₙH₂ₙ₊₂—, —CₙH₂ₙ—, —CₙH₂ₙ₋₂—, where n is more than 1, —S—S—, —RC═N—, —N═CR—, —O═N═C—, —C═N—O—, —O—C(O)—O—, —C(O)—NR—, —NR—C(O)—, —O—C(O)—NR—, —N R—C(O)—O—, —NR¹⁰—C(O)—NR²⁰—, —NR¹⁰—C(S)—NR²⁰—, —NR¹⁰SO₂NR²⁰— where R, R¹⁰ and R²⁰ is selected from hydrogen, straight and/or branched alkyl, alkenyl and alkynyl chains with C₁-C₂₀, natural amino acids, modified amino acids, which may be the same or different from each other. In some embodiments, Linker⁶ is selected from combination of a bond or straight alkyl chain with C₁-C₂₀ containing —O—C(O)—, —C(O)—, or long chain amino acid (LCAA) moiety.

In some embodiments, Linkers¹⁻³ are linkers connecting the functional components of solid support platform. Linkers¹⁻³ are, independently, selected from, but not limited to, combination of a bond or straight and/or branched alkyl, alkenyl and alkynyl chains with C₁-C₂₀ containing aralkyl, aralkenyl, heteroaralkyl, heteroaralkenyl, aralkynyl, heteroarakynyl, —O—, —C(O)—, —NR—, —S—, —S(O)—, —SO₂—, —SO₂NH—, —NHSO₂—, —CₙH₂ₙ₊₂—, —CₙH₂ₙ—, —CₙH₂ₙ₋₂—, where n is more than 1, —S—S—, —RC═N—, —N═CR—, —O═N═C—, —C═N—O—, —O—C(O)—O—, —C(O)—NR—, —NR—C(O)—, —O—C(O)—NR—, —N R—C(O)—O—, —NR¹⁰—C(O)—NR²⁰—, —NR¹⁰—C(S)—NR²⁰—, —NR¹⁰SO₂NR²⁰— where R, R¹⁰ and R²⁰ is selected from hydrogen, straight and/or branched alkyl, alkenyl and alkynyl chains with C₁-C₂₀, natural amino acids, modified amino acids, which may be the same or different from each other.

In some embodiments, Linkers^{a-c} are linkers connecting Linker¹⁻³ to multivalent peptidyl backbone. In some embodiments, Linkers^{a-c} are, independently, selected from, but not limited to, null, —O—, —NR—C(O)—, —C(O)—NR—, —NR¹⁰—C(O)—NR²⁰—, where R, R¹⁰ and R²⁰ is selected from hydrogen, straight and/or branched alkyl, alkenyl and alkynyl chains with C₁-C₂₀, natural amino acids, modified amino acids, which may be the same or different from each other.

In some embodiments, L^{G1}, L^{G2}, and L^{G3} are functionalized ligands selected, independently, from lipophilic groups including, but not limited to, small molecule (such as biotin, fluorescent dyes including, but not limited to, Cy3 or Cy5), proteins, peptides, antibody (such as Brentuximabvedotin and Gemtuzumab ozogamicin), oligosaccharides, nucleic acids, synthetic polymers, carbohydrates, and lipids, which may be the same or different from each other. In some embodiments, L^{G1}, L^{G2}, and L^{G3} are, independently, carbohydrates.

In some embodiments, carbohydrates are selected from, but not limited to, monosaccharides, disaccharides, trisaccharides, tetrasaccharides, or polysaccharides. In some embodiments, monosaccharides are, independently, selected from radicals of allose, altrose, arabinose, cladinose, erythrose, erythrulose, fructose. D-fucitol, L-fucitol, fucosamine, fucose, fuculose, galactosamine. D-galactosaminitol. N-acetyl-galactosamine, galactose, glucosamine. N-acetyl-glucosamine, glucosaminitol, glucose, glucose-6-phosphate, gulose glyceraldehyde. L-glycero-D-mannos-heptose, glycerol, glycerone, gulose, idose, lyxose, mannosamine, mannose, mannose-6-phosphate, psicose, quinovose, quinovosamine, rhamnitol, rhamnosamine, rhamnose, ribose, ribulose, sedoheptulose, sorbose, tagatose, talose, tartaric acid, threose, xylose, or xylulose. In some embodiments, the monosaccharide is in a racemic configuration. In some embodiments, the monosaccharide is in a D- or L-configuration. In some embodiments, the monosaccharide may further be a deoxy sugar (alcoholic hydroxy group replaced by hydrogen), amino sugar (alcoholic hydroxy group replaced by amino group), a thio sugar (alcoholic hydroxy group replaced by thiol), or C—O replaced by C═S. In some embodiments of carbohydrates, the ring oxygen of cyclic form is replaced by sulfur, is a seleno sugar, a telluro sugar, an aza sugar (ring carbon replaced by nitrogen), an imino sugar (ring oxygen replaced by nitrogen), aphosphano sugar (ring oxygen replaced with phosphorus), a phospho sugar (ring carbon replaced with phosphorus), a C-substituted monosaccharide (hydrogen at a non-terminal carbon atom replaced with carbon), an unsaturated monosaccharide, an alditol (carbonyl group replaced with CHOH group), aldonic acid (aldehydic group replaced by carboxy group), a ketoaldonic acid, a uronic acid, or an aldaric acid. In some embodiments, amino sugars include amino monosaccharides. In some embodiments, the carbohydrate is, independently, selected from a galactosamine, glucosamine, mannosamine, fucosamine, quinovosamine, neuraminic acid, muramic acid, lactosediamine, acosamine, bacillosamine, daunosamine, desosamine, forosaminc, garosamine, kanosamine, kansosamine, mycaminose, mycosamine, perosamine, pneumosamine, purpurosamine, or rhodosamine. It understood that the monosaccharide and the like can be further substituted. In some embodiments, the substituent is one or more of an alkyl-alkenyl, alkynyl, aryl, or heteroaryl-carbonyl. In some embodiments, the substituent is an acyl group.

In some embodiments, the terms “disaccharide.” “trisaccharide.” and “polysaccharide” refer to radicals of abequose, acrabose, anucetose, amylopectin, amylose, apiose, arcanose, ascarylose, ascorbic acid, boivinose, cellobiose, cellobiose, cellulose, chacotriose, chalcose, chitin, colitose, cyclodextrin, cymarose, dextrin, 2-deoxyribose, 2deoxyglucose, diginose, digitalose, digitoxose, evalose, evemitrose, fructooligosachharide, galto-oligosaccharide, gentianose, gentiobiose, glucan, glucogen, glycogen, hamamclose, heparin, inulin, isolevoglucosenone, isomaltose, isomaltotriose, isopanose, kojibiose, lactose, lactosamine, lactosediamine, laminarabiose, levoglucosan, levoglucosenone, β-maltose, maltriose, mannan-oligosaccharide, manninotnose, melezitose, melibiose, muramic acid, mycarose, mycinose, neuraminic acid, nigerose, nojirimycin, noviose, oleandrose, panose, paratose, plantcose, primeverose, raffinose, rhodinose, rutinose, sarmentose, sedoheptulose, sedoheptulosan, solatriose, sophorose, stachyose, streptose, sucrose, am-trehalose, trehalosamine, turanose, tyvelose, xylobiose, umbelliferose and the like. Further, it is understood that the “disaccharide”, “trisaccharide” and “polysaccharide” and the like can be further substituted. In some embodiments, the term sisaccharide also includes amino sugars and their derivatives, particularly, a mycaminose derivatized at the C1′ position or a 4 deoxy-3-amino-glucose derivatized at the C6′ position.

In some embodiments, each hydroxyl group of a saccharide, aside from that used for a glycosidic linkage, is substituted with an acyl group.

In some embodiments, lipids are selected from, but not limited to, fatty acids, glycerides, sterols, and their modified derivatives. In some embodiments, fatty acids are carboxylic acids with a long aliphatic chain, which is either saturated or unsaturated and straight or branched chain, of carbon atoms from C₄ to C₂₈. In some embodiments, fatty acids are, but are not limited to, saturated fatty acids such as butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behnic acid, tricoylic acid, lignoceric acid, pentacosylic acid, cerotic acid, carboceric acid, montanic acid, nonacosylic acid, melissic acid, hentriacontylic acid, lacceroic acid, psyllic acid, geddic acid, ceroplastic acid, hexatriacontylic acid, heptatriacontylic acid, octatriacontylic acid, nonatriacontylic acid, tetracontylic acid, octanoic acid, decanoic acid, decadienoic acid, lauroleic acid, laurolinoleic acid, myristovaccenic acid, myristolinoleic acid, myristolinolenic acid, palmitolinolenic acid, palmitidonic acid, α-linolenic acid, stearidonic acid, dihomo-α-linolenic acid, cicosatetraenoic acid, cicosapentaenoic acid, clupanodonic acid, docosahexaenoic acid, 9,12,15,18,21-tetracosapentaenoic acid, 6,9,12,15,18,21-tetracosahexaenoic acid, myristoleic acid, palmitovaccenic acid, α-eleostearic acid, β-eleostearic acid, punicic acid, 7,10,13-octadecatrienoic acid, 9,12,15-cicosatrienoic acid, β-cicosatetraenoic acid, 8-tetradecenoic acid, 12-octadecenoic acid, linoleic acid, linolelaidic acid, γ-linolenic acid, calendic acid, pinolenic acid, dihomo-linoleic acid, dihomo-γ-linolenic acid, arachidonic acid, adrenic acid, osbond acid, palmitoleic acid, vaccenic acid, rumenic acid, paullinic acid, 7,10,13-cisocatrienoic acid, oleic acid, elaidic acid, gongoic acid, crucic acid, nervonic acid, 8,11-eicosadienoic acid, mead acid, sapienic acid, gadoleic acid, 4-hexadecenoic acid, petroselinic acid, 8-eicosenoic acid, bosseopentaenoic acid, cicosapentaenoic acid, ozubondo acid, sardine acid, tetracosanolpentaenoic acid, cervonic acid, herring acid, succinic acid, glutaric acid, adipic acid, pimelic acid, suberic acid, azelaic acid, sebacic acid, brassylic acid, thapsic acid, japanic acid, phellogenic acid, or equisetolic acid.

In some embodiments, glycerides, also known as acyglycerols, are selected from, but are not limited to, monoglycerides containing one of the fatty acids mentioned above, 1,2-diglycerides, or 1,3-diglycerides containing two of the fatty acids mentioned above, which may be the same or different from each other, and triglycerides containing three of the fatty acids mentioned above, which may be the same or different from each other.

In some embodiments, sterols are chemical compounds with formula of C₁₇H₂₈O backbone. In some embodiments, sterols include, but are not limited to, phytosterols such as campesterol, sitosterol and stigmasterol and zoosterols, such as cholesterol, ergosterol, or hopanoids, and their modified derivatives.

In some embodiments, the multivalent peptidyl backbone comprises at least two peptide bonds where multivalency is from two side chains and backbone amine of two amino acids selected from natural amino acids and modified amino acids containing functionalized side chains such as alcohol, phenol, thiol, amine, carboxylic acid, or amide. In some embodiments, the multivalent peptidyl backbone can be extended by elongation with Linker¹⁻³ and Linker^{a-c}.

In some embodiments, provided herein are methods of preparing solid support platforms for the synthesis of macromolecules having the formula described herein.

In some embodiments, the solid support platform includes synthetic steps as shown in FIG. 1. In some embodiments, the method of preparing includes one or more of the following: a) solid support platform is synthesized by peptidyl bond formation and functionalization of linkers and ligands, starting from solid support; b) solid support is connected to Linker⁶ containing modifier P connected to Linker⁴ and amino functional group connected to Linker⁵; and c) multivalent peptidyl backbone is step-by-step elongated by solid phase peptide synthesis and their side chain is modified to the functionalized ligands L^{G1}-L^{G3} by Linker¹⁻³ and Linker^{a-c}.

In some embodiments, the solid support platform includes synthetic steps as shown in FIG. 2. In some embodiments, the method of preparing includes one or more of the following: a) the functionalized ligands containing molecule is synthesized by solid phase peptide synthesis or solution phase peptide synthesis; and b) solid support platform is synthesized by coupling the functionalized ligands containing molecule to solid support.

In some embodiments, the solid support platform includes synthetic steps as shown in FIG. 3. In some embodiments, the method of preparing includes one or more of the following: a) solid support platform containing modifier P is deblocked by trityl deprotection to give the starting point of solid phase oligonucleotide synthesis; b) oligonucleotide is synthesized through automated oligonucleotide synthetic cycles composing of deblocking, coupling, capping, and oxidation; and c) oligonucleotide is processed by post-synthesis procedure comprising of solid support cleavage, deprotection, purification, and, optionally, characterization.

In some embodiments, provided herein are methods of gene silencing, or methods of treatment, comprising administering an effective amount of a compound described herein to a subject in need thereof. The terms “effective amount” and “therapeutically effective amount” refer to an amount of active ingredient, such as a compound described herein, administered to a subject, either as a single dose or as part of a series of doses, which produces a desired effect. In general, the effective amount can be estimated initially either in cell culture assays or in mammalian animal models, for example, in non-human primates, mice, rabbits, dogs, or pigs. The animal model may also be used to determine the appropriate concentration range and route of administration. Such information can then be used to determine useful doses and routes for administration in non-human subjects and human subjects. The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset. Thus, in some embodiments, provided herein are methods of treating a disease in a subject in need thereof, comprising administering to the subject a therapeutically effective amount of a compound or composition provided herein. In some embodiments, the disease may be associated with a genetic disease, which treatment thereof may include activating gene silencing directed to a gene associated with the genetic disease. In some embodiments, provided herein are methods of activating gene silencing in a cell, comprising contacting the cell with an effective amount of the compound or composition of one of claims 1-32, wherein the compound comprises an oligomer comprising one or more monomer units, each monomer unit comprising a C₃₋₆ heterocyclic ring. In some embodiments, the cell is in a subject, e.g., a mammalian subject. In some embodiments, the cell or method is in vitro.

### EXAMPLES

The following examples describe techniques for producing specific and general solid support platforms as described herein.

### General Procedure:

1) Synthesis of solid support platform containing bioconjugation ligands: C3′-amino modifier C7 CPG (hereinafter referred to as CPG nevertheless what attached, 48 μmol/g) is rinsed with DCM and DMF. Then, CPG is subjected to the repeated cycles of alternate N-terminal deprotection and coupling reactions with amino acids to give a multivalent peptidyl backbone ready for ligand coupling reaction. Bioconjugation ligands are attached to linkers by coupling reactions to provide the target solid support platform.

2) Synthesis of functionalized oligonucleotide containing bioconjugation ligands: A functionalized oligonucleotide is synthesized on solid support platform containing tri-GalNAc ligands by automated oligonucleotide solid phase synthesizer. Oligonucleotide containing tri-ligands is synthesized by standard process using phosphoramidite technology on solid support platform. Depending on the scale either a MerMade 12 (Bioautomation) or a Dr.Oligo 48 (Biolytic) is used. All phosphoramidites are purchased from, but not limited to, ChemGenes and Glen Research. All amidites are dissolved in anhydrous acetonitrile and/or DMF in adequate concentration. Deblock solution is selected from, but not limited to, acetic acid, chloroacetic acid, dichloroacetic acid, trichloroacetic acid, or trifluoroacetic acid in an inert solvent (DCM or toluene). Activator solution is selected from, but not limited to, acidic azole catalysts including 1H-tetrazole, 5-ethylthio-1H-tetrazole (ETT) and 2-Benzylthio-1H-tetrazole (BTT) or 4,5-dicyanoimidazole (DCI) or a number of similar compounds which is dissolved in anhydrous acetonitrile in adequate concentration. Capping solution is selected from, but not limited to, a mixture of acetic anhydride and pyridine in THF and N-methylimidazole in acetonitrile. Oxidizing solution is selected from, but not limited to, iodine in water, pyridine and THF and tert-butyl hydroperoxide, (1S)-(+)-(10-camphorsulfonyl)-oxaziridine (CSO). Sulfurization solution is selected from, but not limited to, 3-(dimethylaminomethylidene)amino-3H-1,2,4-dithiazoe-3-thione (DDTT), 3H-1,2-benzodithiol-3-one 1,1-dioxide (Beaucage reagent), or N,N,N′,N′-tetraethylthiuramdisulfide (TETD).

3) Post-synthetic process of functionalized oligonucleotide containing bioconjugation ligands: A functionalized oligonucleotide containing bioconjugation ligands is subsequentially processed with cleavage and deprotection, purification and quantification. Solid support is cleaved and protecting groups of oligonucleotides are deprotected by treatment with a mixture of ammonium hydroxide and/or methylamine (AMA) at ambient temperature. Then, functionalized oligonucleotide containing bioconjugation ligands is purified by oligo purification cartridge (OPC) and/or liquid chromatography utilizing reverse phase (RP) and/or anion exchange (AEX) column. Finally, oligonucleotide is quantified by UV spectrophotometric method using calculated extinction coefficient.

4) Mass data is collected after treatment of solid support platform containing compound with AMA (aqueous ammonium hydroxide:aqueous 40% methyl amine; 1:1 (v/v) mixture) at room temperature for 2 hours followed by neutralization with a mixture of acetic acid and 1 M TEAA (tricthylamine acetate, pH 4.5) (1:5 v/v; final pH 7).

### Example 1: Synthesis of 3′-tri-2-acetamido-2-deoxy-D-galactopyranose tetra-peptidyl Modified Oligonucleotide Using tri-N-acetylchondrosamine tetra-peptidyl CPG

Solid support platform of CPG containing tri-N-acetylchondrosamine tetra-peptidyl modification was synthesized according to the general procedure for synthesis of solid support platform. Representatively, CPG 500 mg was placed in syringe-frit reactor and slowly rinsed and swelled with DCM 3×2.5 mL and DMF 3×2.5 mL for 10 minutes. Then, CPG was treated with 20% piperidine in DMF 3×2.5 mL×30 minutes or 20% 4-methylpiperidine in DMF 3×2.5 mL×30 minutes for the Fmoc deprotection. Collected drainage was used for Fmoc quantification. After rinsing with DCM 3×2.5 mL and DMF 3×2.5 mL for 10 minutes, CPG was treated with a mixture of Fmoc-Phe-OH 28 mg, HATU 27 g, DIPEA 25 μL in DMF 0.36 mL for 60 minutes for the coupling reaction. After draining and rinsing with DCM 3×2.5 mL and DMF 3×2.5 mL for 10 minutes, CPG was treated with a mixture of acetic anhydride/lutidine/THF (1:1:8 v/v/v) 1.25 mL and 16% Methylimidazole/THF 1.25 mL three times for the capping. After draining and rinsing with DCM 3×2.5 mL and DMF 3×2.5 mL, CPG was subjected to the repeated cycles with a mixture of Fmoc-Lys (ivDde)-OH, HATU, DIPEA in DMF, Fmoc-GABA, HATU, DIPEA in DMF, or N-acetylchondrosamine C5 acid, HATU, DIPEA in DMF to afford the corresponding solid support platform. Then, it was used for the synthesis of functionalized oligonucleotide on solid support platform according to the general procedure for the synthesis of functionalized oligonucleotide. Finally, functionalized oligonucleotide containing 3′-tri-2-acetamido-2-deoxy-D-galactopyranose tetra-peptidyl modification was cleaved and deprotected, purified and quantified according to the general procedure for the post-synthetic process to provide the target compound.

Solid support platform

| | |
|---|---|
| SSP Name | Tri-N-acetylchondrosamine tetra-peptidyl CPG |
| Molecular weight | Calculated 1847.0, Measured 1846.9 (after cleavage from solid support) |

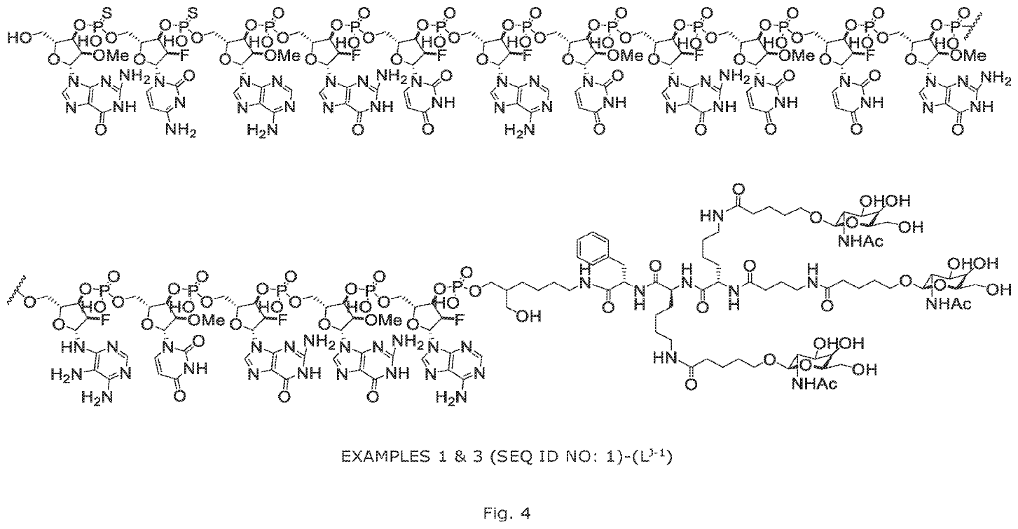

Functionalized macromolecule structure shown in Fig. 4

| | |
|---|---|
| (SEQ ID NO: 1)-(L^{J-1}) | 5′-HO-mG*fC*mAfGfUfAmUfGmUfUmGfAmUfGmGfA-3′-(L^{J-1}) |
| Sequence Name | 3′-Tri-2-acetamido-2-deoxy-D-galactopyranose tetra-peptidyl modified |
| | oligonucleotide |
| Molecular weight | Calculated 6918.1, Measured 6918.1 |

L^{J-1}: (GAL)-GABA-K^{L}-(GAL)-K^{L}-(GAL)-F^{L}-

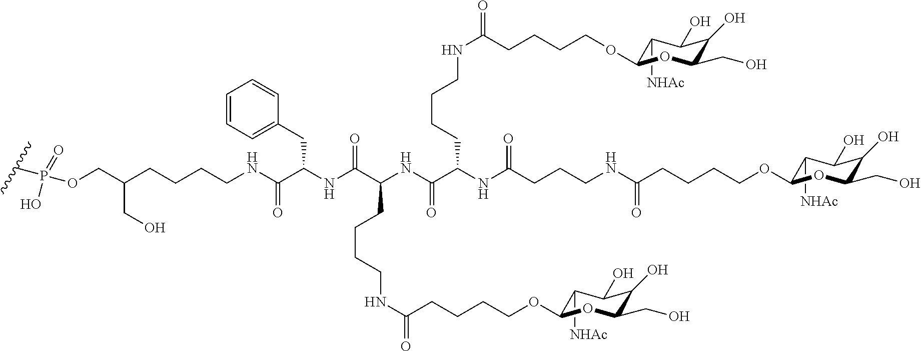

(12S,15S,18S)-1-(((2R,3R,4R,6R)-3-acetamido-4,5-dihydroxy-6-(hydroxymethyl)tetrahydro-2H-pyran-
2-yl)oxy)-12,15-bis(4-(5-(((2R,3R,4R,6R)-3-acetamido-4,5-dihydroxy-6-(hydroxymethyl)tetrahydro-
2H-pyran-2-yl)oxy)pentanamido)butyl)-18-benzyl-25-(hydroxymethyl)-5,10,13,16,19-pentaoxo-
6,11,14,17,20-pentaazahexacosan-26-yl hydroxyphosphoryl
Functionalized macromolecule name:
(12S,15S,18S)-1-(((2R,3R,4R,6R)-3-acetamido-4,5-dihydroxy-6-(hydroxymethyl)tetrahydro-2H-pyran-
2-yl)oxy)-12,15-bis(4-(5-(((2R,3R,4R,6R)-3-acetamido-4,5-dihydroxy-6-(hydroxymethyl)tetrahydro-
2H-pyran-2-yl)oxy)pentanamido)butyl)-18-benzyl-25-(hydroxymethyl)-5,10,13,16,19-pentaoxo-
6,11,14,17,20-pentaazahexacosan-26-yl hydroxyphosphoryl-2′-deoxy-fluoro-adenylyl-(3′→5′)-2′-O-
methyl-guanylyl-(3′→5′)-2-deoxy-fluoro-guanylyl-(3′→5′)-2′-O-methyl-uridylyl-(3′→5′)-2′-deoxy-
fluoro-adenylyl-(3′→5′)-2′-O-methyl-guanylyl-(3′→5′)-2′-deoxy-fluoro-uridylyl-(3′→5′)-2′-O-
methyl-uridylyl-(3′→5′)-2-deoxy-fluoro-guanylyl-(3′→5′)-2′-O-methyl-uridylyl-(3′→5′)-2′-deoxy-
fluoro-adenylyl-(3′→5′)-2′-deoxy-fluoro-uridylyl-(3′→5′)-2-deoxy-fluoro-guanylyl-(3′→5′)-2′-O-
methyl-adenylyl-(3′→5′)-P(R,S)-2′-deoxy-fluoro-P-thiocytidylyl-(3′→5′)-P(R,S)-2′-O-methyl-P-
thioguanosine

### Example 2: Synthesis of 3′-tri-2-acetamido-2-deoxy-D-galactopyranose tri-peptidyl Modified Oligonucleotide Using tri-N-acetylchondrosamine tri-peptidyl CPG

Solid support platform of CPG containing tri-N-acetylchondrosamine tri-peptidyl modification was synthesized according to the general procedure for synthesis of solid support platform. Then, it was used for the synthesis of functionalized oligonucleotide on solid support platform according to the general procedure for the synthesis of functionalized oligonucleotide. Finally, functionalized oligonucleotide containing 3′-tri-2-acetamido-2-deoxy-D-galactopyranose tri-peptidyl modification was cleaved and deprotected, purified and quantified according to the general procedure for the post-synthetic process to provide the target compound.

Solid support platform

| | |
|---|---|
| SSP Name | Tri-N-acetylchondrosamine tri-peptidyl CPG |
| Molecular weight | Calculated 1761.9, Measured 1761.8 (after cleavage from solid support) |

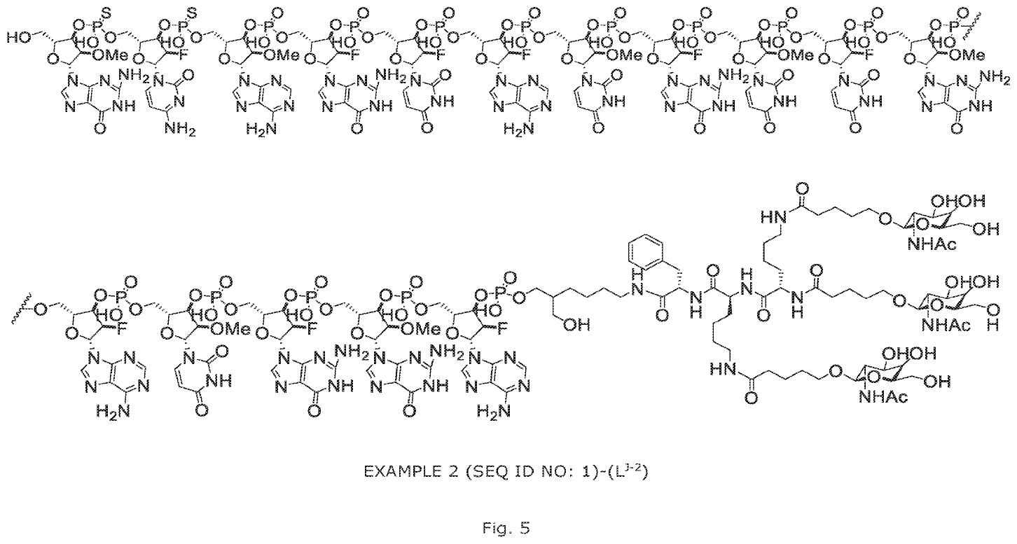

Functionalized macromolecule structure shown in Fig. 5

| | |
|---|---|
| (SEQ ID NO: 1)-(L^{J-2}) | 5′-HO-mG*fC*mAfGfUfAmUfGmUfUmGfAmUfGmGfA-3′-(L^{J-2}) |
| Sequence Name | 3′-Tri-2-acetamido-2-deoxy-D-galactopyranose tri-peptidyl modified |
| | oligonucleotide |
| Molecular weight | Calculated 6833.0, Measured 6833.2 |

L^{J-2}: (GAL)-K^{L}(GAL)-K^{L}(GAL)-F^{L}-

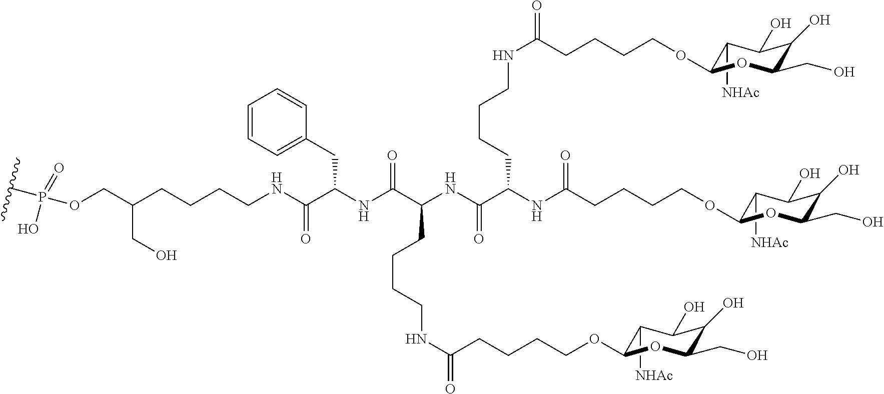

(11S,14S,17S)-1-(((2R,3R,4R,6R)-3-acetamido-4,5-dihydroxy-6-(hydroxymethyl)tetrahydro-2H-pyran-
2-yl)oxy)-11-(5-(((2R,3R,4R,6R)-3-acetamido-4,5-dihydroxy-6-(hydroxymethyl)tetrahydro-2H-pyran-
2-yl)oxy)pentanamido)-14-(4-(5-(((2R,3R,4R,6R)-3-acetamido-4,5-dihydroxy-6-
(hydroxymethyl)tetrahydro-2H-pyran-2-yl)oxy)pentanamido)butyl)-17-benzyl-24-(hydroxymethyl)-
5,12,15,18-tetraoxo-6,13,16,19-tetraazapentacosan-25-yl hydroxyphosphoryl
Functionalized macromolecule name:
(11S,14S,17S)-1-(((2R,3R,4R,6R)-3-acetamido-4,5-dihydroxy-6-(hydroxymethyl)tetrahydro-2H-pyran-
2-yl)oxy)-11-(5-(((2R,3R,4R,6R)-3-acetamido-4,5-dihydroxy-6-(hydroxymethyl)tetrahydro-2H-pyran-
2-yl)oxy)pentanamido)-14-(4-(5-(((2R,3R,4R,6R)-3-acetamido-4,5-dihydroxy-6-
(hydroxymethyl)tetrahydro-2H-pyran-2-yl)oxy)pentanamido)butyl)-17-benzyl-24-(hydroxymethyl)-
5,12,15,18-tetraoxo-6,13, 16, 19-tetraazapentacosan-25-yl hydroxyphosphoryl-2′-deoxy-fluoro-adenylyl-
(3′→5′)-2′-O-methyl-guanylyl-(3′→5′)-2-deoxy-fluoro-guanylyl-(3′→5′)-2′-O-methyl-uridylyl-
(3′→5′)-2′-deoxy-fluoro-adenylyl-(3′→5′)-2′-O-methyl-guanylyl-(3′→5′)-2′-deoxy-fluoro-uridylyl-
(3′→5′)-2′-O-methyl-uridylyl-(3′→5′)-2-deoxy-fluoro-guanylyl-(3′→5′)-2′-O-methyl-uridylyl-
(3′→5′)-2′-deoxy-fluoro-adenylyl-(3′→5′)-2′-deoxy-fluoro-uridylyl-(3′→5′)-2-deoxy-fluoro-guanylyl-
(3′→5′)-2′-O-methyl-adenylyl-(3′→5′)-P(R,S)-2′-deoxy-fluoro-P-thiocytidylyl-(3′→5′)-P(R,S)-2′-O-
methyl-P-thioguanosine

### Example 3: Synthesis of 3′-tri-2-acetamido-2-deoxy-D-galactopyranose tetra-peptidyl Modified Oligonucleotide Using tri-N-acetylchondrosamine tetra-peptidyl Polystyrene

Solid support platform of PS containing 3′-tri-2-acetamido-2-deoxy-D-galactopyranose tetra-peptidyl modification was synthesized according to the general procedure for synthesis of solid support platform. Then, it was used for the synthesis of functionalized oligonucleotide on solid support platform according to the general procedure for the synthesis of functionalized oligonucleotide. Finally, functionalized oligonucleotide containing 3′-tri-2-acetamido-2-deoxy-D-galactopyranose tetra-peptidyl modification was cleaved and deprotected, purified and quantified according to the general procedure for the post-synthetic process to provide the target compound.

Solid support platform

| | |
|---|---|
| SSP Name | Tri-N-acetylchondrosamine tetra-peptidyl polystyrene |
| Molecular weight | Calculated 1847.0, Measured 1847.0 (after cleavage from solid support) |

Functionalized macromolecule structure shown in FIG. 4

| | |
|---|---|
| (SEQ ID NO: 1)-(L^{J-1}) | 5′-HO-mG*fC*mAfGfUfAmUfGmUfUmGfAmUfGmGfA-3′-(L^{J-1}) |
| Sequence Name | 3′-Tri-2-acetamido-2-deoxy-D-galactopyranose tetra-peptidyl modified |
| | oligonucleotide |
| Molecular weight | Calculated 6918.1, Measured 6917.8 |

### Example 4: Synthesis of 3-tri-2-D-mannopyranose tetra-peptidyl Modified Oligonucleotide Using tri-mannose tetra-peptidyl CPG

Solid support platform of CPG containing tri-mannose tetra-peptidyl modification was synthesized according to the general procedure for synthesis of solid support platform. Then, it was used for the synthesis of functionalized oligonucleotide on solid support platform according to the general procedure for the synthesis of functionalized oligonucleotide. Finally, functionalized oligonucleotide containing 3′-tri-2-D-mannopyranose tetra-peptidyl modification was cleaved and deprotected, purified and quantified according to the general procedure for the post-synthetic process to provide the target compound.

Solid support platform

| | |
|---|---|
| SSP Name | Tri-mannose tetra-peptidyl CPG |
| Molecular weight | Calculated 1725.0, Measured 1724.2 (after cleavage from solid support) |

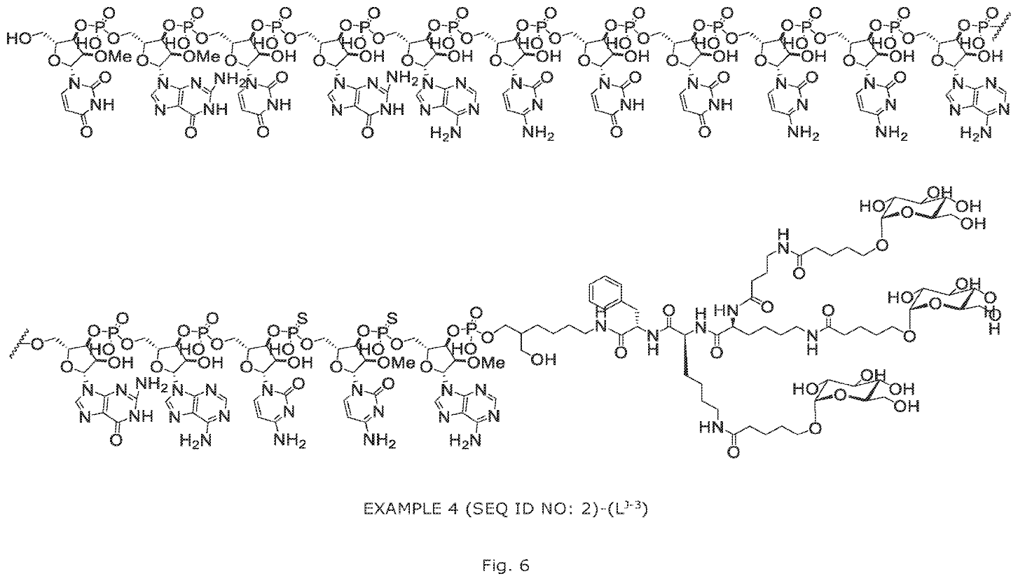

Functionalized macromolecule structure shown in Fig. 6

| | |
|---|---|
| (SEQ ID NO: 2)-(L^{J-3}) | 5′-HO-mUmGUGACUUCCAGAC*mC*mA-3′-(L^{J-3}) |
| Sequence Name | 3′-Tri-2-D-mannopyranose tetra-peptidyl modified oligonucleotide |
| Molecular weight | Calculated 6613.9, Measured 6613.9 |

L^{J-3}: (MAN)-(GABA)-K^{L}(MAN)-K^{L}(MAN)-F^{L}-

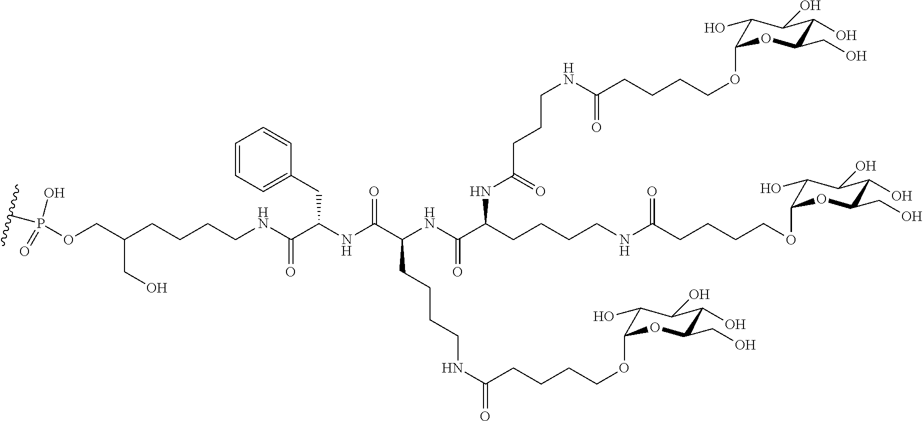

(12S, 15S,18S)-18-benzyl-25-(hydroxymethyl)-5,10,13,16,19-pentaoxo-1-(((2S,3R,4S,5S,6R)-3,4,5-
trihydroxy-6-(hydroxymethyl)tetrahydro-2H-pyran-2-yl)oxy)-12,15-bis(4-(5-(((2S,3R,4S,5S,6R)-3,4,5-
trihydroxy-6-(hydroxymethyl)tetrahydro-2H-pyran-2-yl)oxy)pentanamido)butyl)-6,11,14,17,20-
pentaazahexacosan-26-yl hydroxyphosphoryl
Functionalized macromolecule name:
(12S,15S, 18S)-18-benzyl-25-(hydroxymethyl)-5,10,13,16,19-pentaoxo-1-(((2S,3R,4S,5S,6R)-3,4,5-
trihydroxy-6-(hydroxymethyl)tetrahydro-2H-pyran-2-yl)oxy)-12,15-bis(4-(5-(((2S,3R,4S,5S,6R)-3,4,5-
trihydroxy-6-(hydroxymethyl)tetrahydro-2H-pyran-2-yl)oxy)pentanamido)butyl)-6,11,14,17,20-
pentaazahexacosan-26-yl hydroxyphosphoryl -2′-O-methyl-adenylyl-(3′→5′)-P(R,S)-2′-O-methyl-P-
thiocytidylyl-(3′→5′)-(P-R,S)-P-thiocytidylyl-(3′→5′)adenylyl-(3′→5′)-guanylyl-(3′→5′)-adenylyl-
(3′→5′)-cytidylyl-(3′→5′)-cytidylyl-(3′→5′)-uridylyl-(3′→5′)-uridylyl-(3′ →5′)-cytidylyl-(3′→5′)-
adenylyl-(3′→5′)-guanylyl-(3′→5′)-uridylyl-(3′→5′)-2′-O-methyl-guanylyl-(3′→5′)-2′-O-methyl-
uridine

### Example 5: Synthesis of 3′-hexa-2-D-mannopyranose penta-peptidyl mono-divalent Modified Oligonucleotide Using hexa-mannose penta-peptidyl Divalent CPG

Solid support platform of CPG containing hexa-mannose penta-peptidyl divalent modification was synthesized according to the general procedure for synthesis of solid support platform. Then, it was used for the synthesis of functionalized oligonucleotide on solid support platform according to the general procedure for the synthesis of functionalized oligonucleotide. Finally, functionalized oligonucleotide containing 3′-hexa-2-D-mannopyranose penta-peptidyl mono-divalent modification was cleaved and deprotected, purified and quantified according to the general procedure for the post-synthetic process to provide the target compound.

Solid support platform

| | |
|---|---|
| SSP Name | Hexa-mannose penta-peptidyl divalent CPG |
| Molecular weight | Calculated 2981.4, Measured 2981.4 (after cleavage from solid support) |

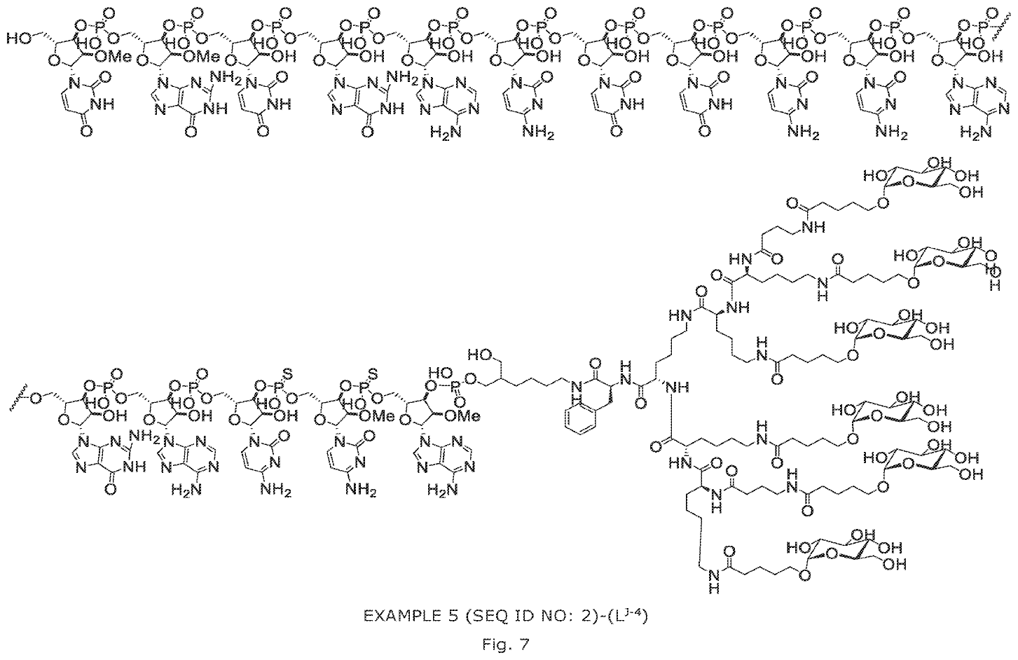

Functionalized macromolecule structure shown in Fig. 7

| | |
|---|---|
| (SEQ ID NO: 2)-(L^{J-4}) | 5′-HO-mUmGUGACUUCCAGAC*mC*mA-3′-(L^{J-4}) |
| Sequence Name | 3′-Hexa-2-D-mannopyranose penta-peptidyl divalent modified oligonucleotide |
| Molecular weight | Calculated 7856.0, Measured 7856.6 |

L^{J-4}: ((MAN)-(GABA)-K^{L}(MAN)-K^{L}(MAN))-K^{L}((MAN)-(GABA)-K^{L}(MAN)-K^{L}(MAN))-F^{L}-

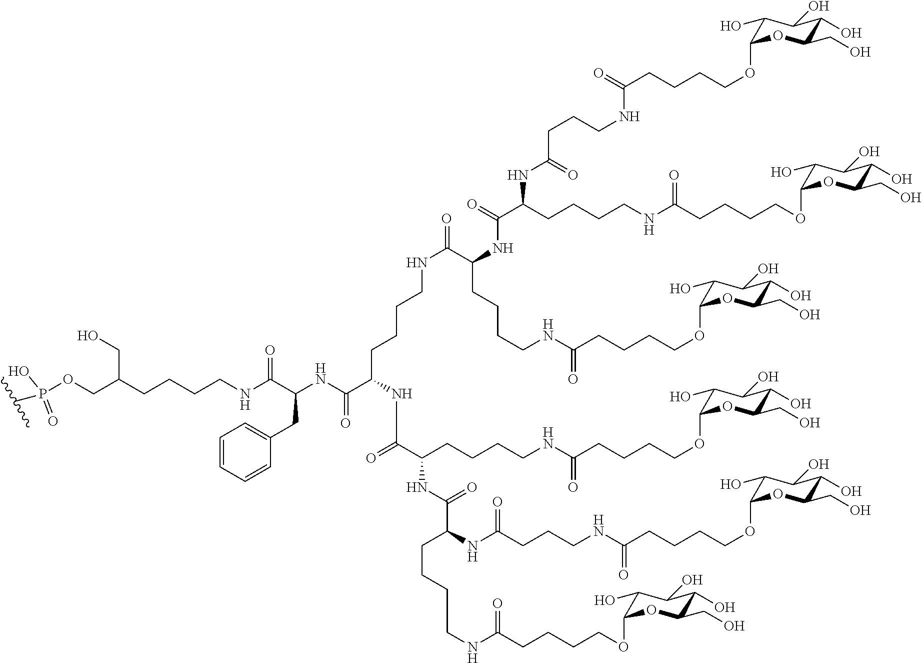

(12S, 15S,22S,25S)-25-benzyl-32-(hydroxymethyl)-5,10,13,16,23,26-hexaoxo-1-(((2S,3R,4S,5S,6R)-
3,4,5-trihydroxy-6-(hydroxymethyl)tetrahydro-2H-pyran-2-yl)oxy)-22-((S)-6-(5-(((2S,3R,4S,5S,6R)-
3,4,5-trihydroxy-6-(hydroxymethyl)tetrahydro-2H-pyran-2-yl)oxy)pentanamido)-2-((S)-6-(5-
(((2S,3R,4S,5S,6R)-3,4,5-trihydroxy-6-(hydroxymethyl)tetrahydro-2H-pyran-2-yl)oxy)pentanamido)-2-
(4-(5-(((2S,3R,4S,5S,6R)-3,4,5-trihydroxy-6-(hydroxymethyl)tetrahydro-2H-pyran-2-
yl)oxy)pentanamido)butanamido)hexanamido)hexanamido)-12,15-bis(4-(5-(((2S,3R,4S,5S,6R)-3,4,5-
trihydroxy-6-(hydroxymethyl)tetrahydro-2H-pyran-2-yl)oxy)pentanamido)butyl)-6,11,14,17,24,27-
hexaazatritriacontan-33-yl hydroxyphosphoryl
Functionalized macromolecule name:
(12S,15S,22S,25S)-25-benzyl-32-(hydroxymethyl)-5,10,13,16,23,26-hexaoxo-1-(((2S,3R,4S,5S,6R)-
3,4,5-trihydroxy-6-(hydroxymethyl)tetrahydro-2H-pyran-2-yl)oxy)-22-((S)-6-(5-(((2S,3R,4S,5S,6R)-
3,4,5-trihydroxy-6-(hydroxymethyl)tetrahydro-2H-pyran-2-yl)oxy)pentanamido)-2-((S)-6-(5-
(((2S,3R,4S,5S,6R)-3,4,5-trihydroxy-6-(hydroxymethyl)tetrahydro-2H-pyran-2-yl)oxy)pentanamido)-2-
(4-(5-(((2S,3R,4S,5S,6R)-3,4,5-trihydroxy-6-(hydroxymethyl)tetrahydro-2H-pyran-2-
yl)oxy)pentanamido)butanamido)hexanamido)hexanamido)-12,15-bis(4-(5-(((2S,3R,4S,5S,6R)-3,4,5-
trihydroxy-6-(hydroxymethyl)tetrahydro-2H-pyran-2-yl)oxy)pentanamido)butyl)-6,11,14,17,24,27-
hexaazatritriacontan-33-yl hydroxyphosphoryl-2′-O-methyl-adenylyl-(3′→5′)-P(R,S)-2′-O-methyl-P-
thiocytidylyl-(3′→5′)-(P-R,S)-P-thiocytidylyl-(3′→5′)adenylyl-(3′→5′)-guanylyl-(3′→5′)-adenylyl-
(3′→5′)-cytidylyl-(3′→5′)-cytidylyl-(3′→5′)-uridylyl-(3′→5′)-uridylyl-(3′→5′)-cytidylyl-(3′→5′)-
adenylyl-(3′→5′)-guanylyl-(3′→5′)-uridylyl-(3′→5′)-2′-O-methyl-guanylyl-(3′→5′)-2′-O-methyl-
uridine

### Example 6: Synthesis of 3′-tri-2-D-mannopyranose tetra-peptidyl 5′-tri-D-mannopyranose tetra-peptidyl Modified Oligonucleotide Using tri-mannose tetra-peptidyl CPG

Solid support platform of CPG containing tri-mannose tetra-peptidyl modification was synthesized according to the general procedure for synthesis of solid support platform. Then, it was used for the synthesis of functionalized oligonucleotide on solid support platform according to the general procedure for the synthesis of functionalized oligonucleotide. Finally, functionalized oligonucleotide containing 3′-tri-2-D-mannopyranose tetra-peptidyl 5′-tri-D-mannospyranose tetra-peptidyl modification was cleaved and deprotected, purified and quantified according to the general procedure for the post-synthetic process to provide the target compound.

Solid support platform

| | |
|---|---|
| SSP Name | Tri-mannose tetra-peptidyl CPG |
| Molecular weight | Calculated 1725.0, Measured 1724.2 (after cleavage from solid support) |

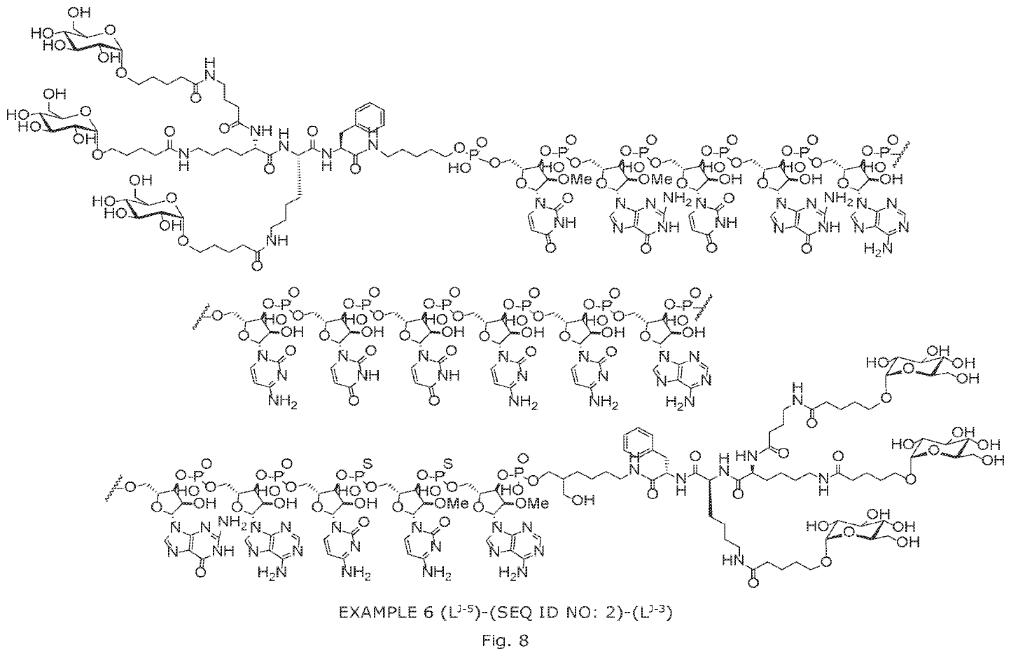

Functionalized macromolecule structure shown in Fig. 8

| | |
|---|---|
| (L^{J-5})-(SEQ ID NO: 2)-(L^{J-3}) | (L^{J-5})-5′-mUmGUGACUUCCAGAC*mC*mA-3′-(L^{J-3}) |
| Sequence Name | 3′-Tri-2-D-mannopyranose tetra-peptidyl 5′-tri-2-D-mannopyranose tetra- |
| | peptidyl modified oligonucleotide |
| Molecular weight | Calculated 8053.5 |

L^{J-5}

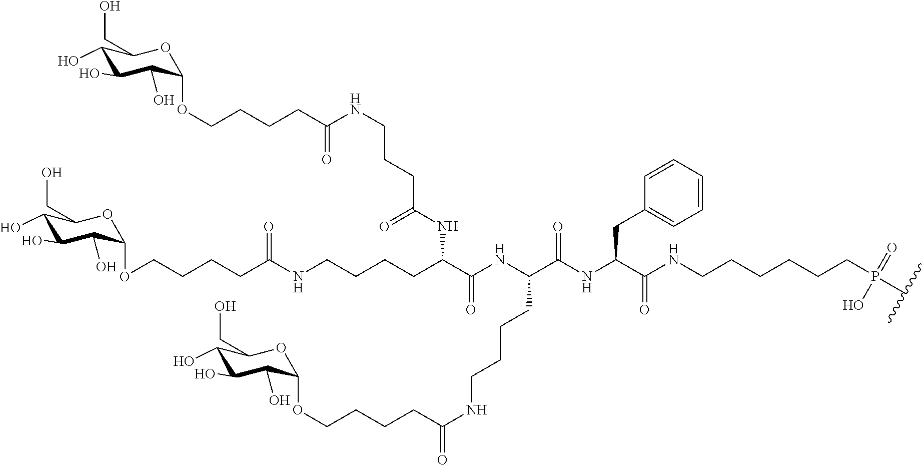

(8S,11S, 14S)-8-benzyl-7,10,13,16,21-pentaoxo-25-(((2S,3R,4S,5S,6R)-3,4,5-trihydroxy-6-
(hydroxymethyl)tetrahydro-2H-pyran-2-yl)oxy)-11,14-bis(4-(5-(((2S,3R,4S,5S,6R)-3,4,5-trihydroxy-6-
(hydroxymethyl)tetrahydro-2H-pyran-2-yl)oxy)pentanamido)butyl)-6,9,12,15,20-pentaazapentacosyl
hydroxyphosphoryl
Functionalized macromolecule name:
(12S,15S,18S)-18-benzyl-5,10,13,16,19-pentaoxo-1-(((2S,3R,4S,5S,6R)-3,4,5-trihydroxy-6-
(hydroxymethyl)tetrahydro-2H-pyran-2-yl)oxy)-12,15-bis(4-(5-(((2S,3R,4S,5S,6R)-3,4,5-trihydroxy-6-
(hydroxymethyl)tetrahydro-2H-pyran-2-yl)oxy)pentanamido)butyl)-6,11,14,17,20-pentaazahexacosan-
26-yl) hydroxyphosphoryl-2′-O-methyl-adenylyl-(3′→5′)-P(R,S)-2′-O-methyl-P-thiocytidylyl-(3′→5′)-
(P-R,S)-P-thiocytidylyl-(3′→5′)adenylyl-(3′→5′)-guanylyl-(3′→5′)-adenylyl-(3′→5′)-cytidylyl-
(3′→5′)-cytidylyl-(3′→5′)-uridylyl-(3′→5′)-uridylyl-(3′→5′)-cytidylyl-(3′→5′)-adenylyl-(3′→5′)-
guanylyl-(3′→5′)-uridylyl-(3′→5′)-2′-O-methyl-guanylyl-(3′→5′)-2′-O-methyl-uridinyl
((8S,11S,14S)-8-benzyl-7,10,13,16,21-pentaoxo-25-(((2S,3R,4S,5S,6R)-3,4,5-trihydroxy-6-
(hydroxymethyl)tetrahydro-2H-pyran-2-yl)oxy)-11,14-bis(4-(5-(((2S,3R,4S,5S,6R)-3,4,5-trihydroxy-6-
(hydroxymethyl)tetrahydro-2H-pyran-2-yl)oxy)pentanamido)butyl)-6,9,12,15,20-pentaazapentacosyl)-
hydrogen phosphate

### Example 7: Synthesis of 3-hexa-2-D-mannopyranose penta-peptidyl Tri-Divalent Modified Oligonucleotide Using hexa-mannose penta-peptidyl Tri-Divalent CPG

Solid support platform of CPG containing hexa-mannose penta-peptidyl tri-divalent modification was synthesized according to the general procedure for synthesis of solid support platform. Then, it was used for the synthesis of functionalized oligonucleotide on solid support platform according to the general procedure for the synthesis of functionalized oligonucleotide. Finally, functionalized oligonucleotide containing 3′-hexa-2-D-mannopyranose penta-peptidyl tri-divalent modification was cleaved and deprotected, purified and quantified according to the general procedure for the post-synthetic process to provide the target compound.

Solid support platform

| | |
|---|---|
| SSP Name | Hexa-mannose penta-peptidyl tri-divalent CPG |
| Molecular weight | Calculated 3065.4, Measured 3064.6 (after cleavage from solid support) |

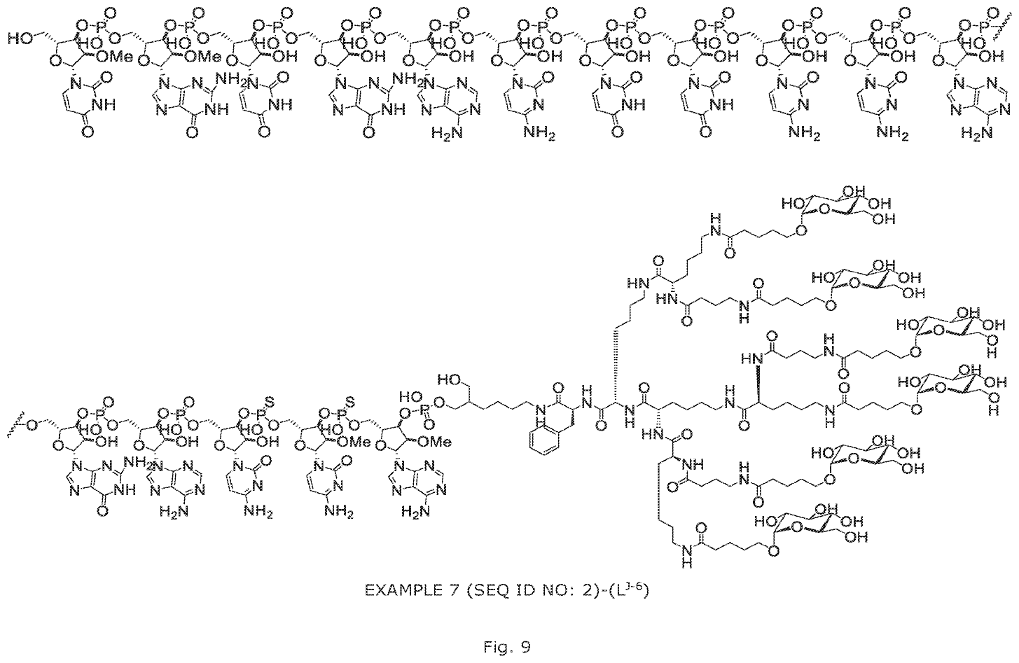

Functionalized macromolecule structure shown in Fig. 9

| | |
|---|---|
| (SEQ ID NO: 2)-(L^{J-6}) | 5′-HO-mUmGUGACUUCCAGAC*mC*mA-3′-(L^{J-6}) |
| Sequence Name | 3′-Hexa-2-D-mannopyranose penta-peptidyl divalent modified oligonucleotide |
| Molecular weight | Calculated 7955.4 |

L^{J-6}: (MAN)-(GABA)-K^{L}(MAN)-K^{L}((MAN)(GABA)-K^{L}(MAN))-K^{L}((MAN)(GABA)-K^{L}(MAN))-F^{L}-

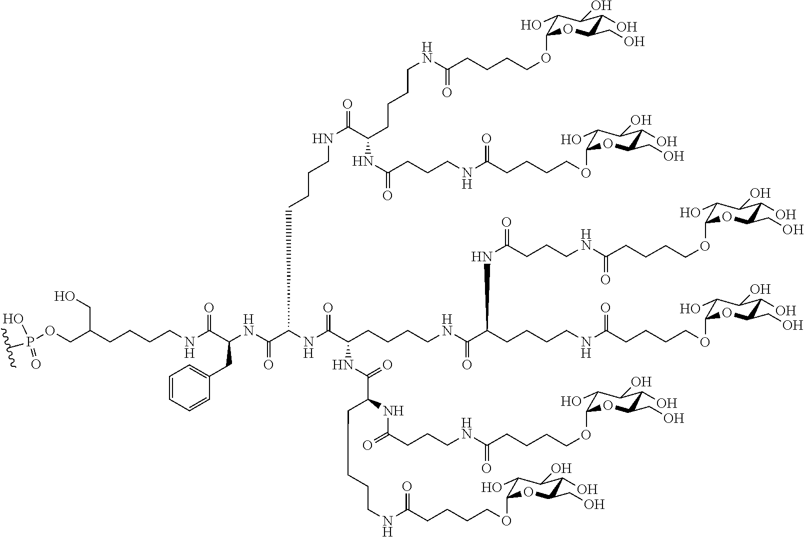

(12S, 19S,22S,25S)-25-benzyl-32-(hydroxymethyl)-5,10,13,20,23,26-hexaoxo-1-(((2S,3R,4S,5S,6R)-
3,4,5-trihydroxy-6-(hydroxymethyl)tetrahydro-2H-pyran-2-yl)oxy)-19-((S)-6-(5-(((2S,3R,4S,5S,6R)-
3,4,5-trihydroxy-6-(hydroxymethyl)tetrahydro-2H-pyran-2-yl)oxy)pentanamido)-2-(4-(5-
(((2S,3R,4S,5S,6R)-3,4,5-trihydroxy-6-(hydroxymethyl)tetrahydro-2H-pyran-2-
yl)oxy)pentanamido)butanamido)hexanamido)-22-(4-((S)-6-(5-(((2S,3R,4S,5S,6R)-3,4,5-trihydroxy-6-
(hydroxymethyl)tetrahydro-2H-pyran-2-yl)oxy)pentanamido)-2-(4-(5-(((2S,3R,4S,5S,6R)-3,4,5-
trihydroxy-6-(hydroxymethyl)tetrahydro-2H-pyran-2-
yl)oxy)pentanamido)butanamido)hexanamido)butyl)-12-(4-(5-(((2S,3R,4S,5S,6R)-3,4,5-trihydroxy-6-
(hydroxymethyl)tetrahydro-2H-pyran-2-yl)oxy)pentanamido)butyl)-6,11,14,21,24,27-
hexaazatritriacontan-33-yl hydroxyphosphoryl
Functionalized macromolecule name:
(12S,19S,22S,25S)-25-benzyl-32-(hydroxymethyl)-5,10,13,20,23,26-hexaoxo-1-(((2S,3R,4S,5S,6R)-
3,4,5-trihydroxy-6-(hydroxymethyl)tetrahydro-2H-pyran-2-yl)oxy)-19-((S)-6-(5-(((2S,3R,4S,5S,6R)-
3,4,5-trihydroxy-6-(hydroxymethyl)tetrahydro-2H-pyran-2-yl)oxy)pentanamido)-2-(4-(5-
(((2S,3R,4S,5S,6R)-3,4,5-trihydroxy-6-(hydroxymethyl)tetrahydro-2H-pyran-2-
yl)oxy)pentanamido)butanamido)hexanamido)-22-(4-((S)-6-(5-(((2S,3R,4S,5S,6R)-3,4,5-trihydroxy-6-
(hydroxymethyl)tetrahydro-2H-pyran-2-yl)oxy)pentanamido)-2-(4-(5-(((2S,3R,4S,5S,6R)-3,4,5-
trihydroxy-6-(hydroxymethyl)tetrahydro-2H-pyran-2-
yl)oxy)pentanamido)butanamido)hexanamido)butyl)-12-(4-(5-(((2S,3R,4S,5S,6R)-3,4,5-trihydroxy-6-
(hydroxymethyl)tetrahydro-2H-pyran-2-yl)oxy)pentanamido)butyl)-6,11,14,21,24,27-
hexaazatritriacontan-33-yl hydroxyphosphoryl-2′-O-methyl-adenylyl-(3′→5′)-P(R,S)-2′-O-methyl-P-
thiocytidylyl-(3′→5′)-(P-R,S)-P-thiocytidylyl-(3′→5′)adenylyl-(3′→5′)-guanylyl-(3′→5′)-adenylyl-
(3′→5′)-cytidylyl-(3′→5′)-cytidylyl-(3′→5′)-uridylyl-(3′→5′)-uridylyl-(3′→5′)-cytidylyl-(3′→5′)-
adenylyl-(3′→5′)-guanylyl-(3′→5′)-uridylyl-(3′→5′)-2′-O-methyl-guanylyl-(3′→5′)-2′-O-methyl-
uridine

### Example 8: Synthesis of 3′-tri-2-acetamido-2-deoxy-D-galactopyranose di-peptidyl Modified Oligonucleotide

Solid support platform of CPG containing tri-N-acetylchondrosamine di-peptidyl (L-Lys-L-Lys) modification was synthesized according to the general procedure for synthesis of solid support platform. Then, it was used for the synthesis of functionalized oligonucleotide on solid support platform according to the general procedure for the synthesis of functionalized oligonucleotide. Finally, functionalized oligonucleotide containing 3′-tri-2-acetamido-2-deoxy-D-galactopyranose di-peptidyl modification was cleaved and deprotected, purified and quantified according to the general procedure for the post-synthetic process to provide the target compound.

Solid support platform

| | |
|---|---|
| SSP Name | Tri-N-acetylchondrosamine di-peptidyl CPG |
| Molecular weight | Calculated 1701.0, Measured 1700.3 |

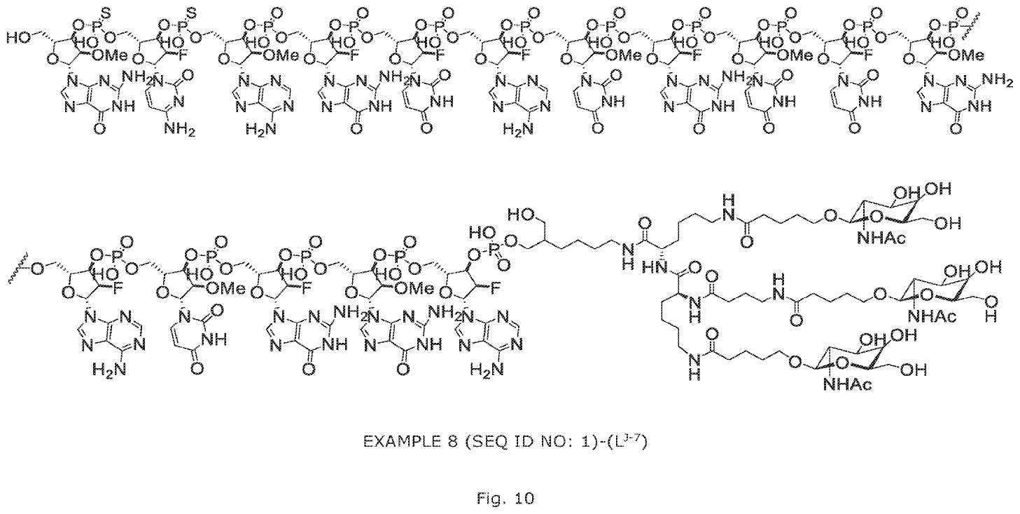

Functionalized macromolecule structure shown in Fig. 10

| | |
|---|---|
| (SEQ ID NO: 1)-(L^{J-7}) | 5′-HO-mG*fC*mAfGfUfAmUfGmUfUmGfAmUfGmGfA-3′-(L^{J-7}) |
| Sequence Name | 3′-Tri-2-acetamido-2-deoxy-D-galactopyranose di-peptidyl modified |
| | oligonucleotide |
| Molecular weight | Calculated 6770.9, Measured 6771.1 |

L^{J-7}: (GAL)-(GABA)-K^{L}(GAL)-K^{L}(GAL)-

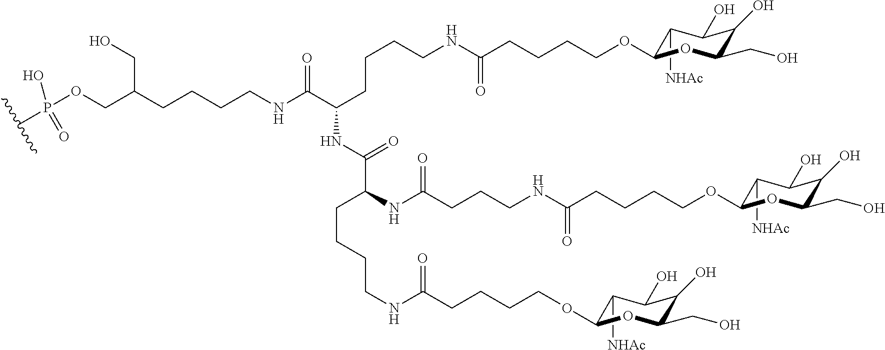

(12S,15S)-1-(((2R,3R,4R,6R)-3-acetamido-4,5-dihydroxy-6-(hydroxymethyl)tetrahydro-2H-pyran-2-
yl)oxy)-12,15-bis(4-(5-(((2R,3R,4R,6R)-3-acetamido-4,5-dihydroxy-6-(hydroxymethyl)tetrahydro-2H-
pyran-2-yl)oxy)pentanamido)butyl)-22-(hydroxymethyl)-5,10,13,16-tetraoxo-6,11,14,17-
tetraazatricosan-23-yl hydroxyphosphoryl
Functionalized macromolecule name:
(12S,15S)-1-(((2R,3R,4R,6R)-3-acetamido-4,5-dihydroxy-6-(hydroxymethyl)tetrahydro-2H-pyran-2-
yl)oxy)-12,15-bis(4-(5-(((2R,3R,4R,6R)-3-acetamido-4,5-dihydroxy-6-(hydroxymethyl)tetrahydro-2H-
pyran-2-yl)oxy)pentanamido)butyl)-22-(hydroxymethyl)-5,10,13,16-tetraoxo-6,11,14,17-
tetraazatricosan-23-yl hydroxyphosphoryl-2′-deoxy-fluoro-adenylyl-(3′→5′)-2′-O-methyl-guanylyl-
(3′→5′)-2-deoxy-fluoro-guanylyl-(3′→5′)-2′-O-methyl-uridylyl-(3′→5′)-2′-deoxy-fluoro-adenylyl-
(3′→5′)-2′-O-methyl-guanylyl-(3′→5′)-2′-deoxy-fluoro-uridylyl-(3′→5′)-2′-O-methyl-uridylyl-
(3′→5′)-2-deoxy-fluoro-guanylyl-(3′→5′)-2′-O-methyl-uridylyl-(3′→5′)-2′-deoxy-fluoro-adenylyl-
(3′→5′)-2′-deoxy-fluoro-uridylyl-(3′→5′)-2-deoxy-fluoro-guanylyl-(3′→5′)-2′-O-methyl-adenylyl-
(3′→5′)-P(R,S)-2′-deoxy-fluoro-P-thiocytidylyl-(3′→5′)-P(R,S)-2′-O-methyl-P-thioguanosine

### Example 9: Synthesis of 3-tri-2-acetamido-2-deoxy-D-galactopyranose di-peptidyl Modified Oligonucleotide

Solid support platform of CPG containing tri-N-acetylchondrosamine di-peptidyl (D-Lys-L-Lys) modification was synthesized according to the general procedure for synthesis of solid support platform. Then, it was used for the synthesis of functionalized oligonucleotide on solid support platform according to the general procedure for the synthesis of functionalized oligonucleotide. Finally, functionalized oligonucleotide containing 3′-tri-2-acetamido-2-deoxy-D-galactopyranose di-peptidyl modification was cleaved and deprotected, purified and quantified according to the general procedure for the post-synthetic process to provide the target compound.

Solid support platform

| | |
|---|---|
| SSP Name | Tri-N-acetylchondrosamine di-peptidyl CPG |
| Molecular weight | Calculated 1701.0, Measured 1700.3 |

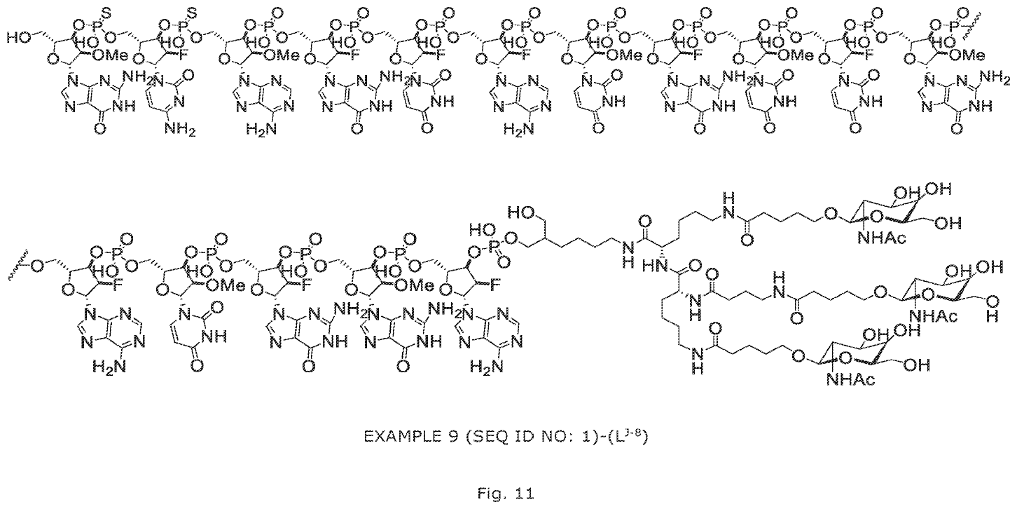

Functionalized macromolecule structure shown in Fig. 11

| | |
|---|---|
| (SEQ ID NO: 1)-(L^{J-8}) | 5′-HO-mG*fC*mAfGfUfAmUfGmUfUmGfAmUfGmGfA-3′-(L^{J-8}) |
| Sequence Name | 3′-Tri-2-acetamido-2-deoxy-D-galactopyranose di-peptidyl modified |
| | oligonucleotide |
| Molecular weight | Calculated 6770.9, Measured 6771.0 |

L^{J-8}: (GAL)-(GABA)-K^{D}(GAL)-K^{L}(GAL)-

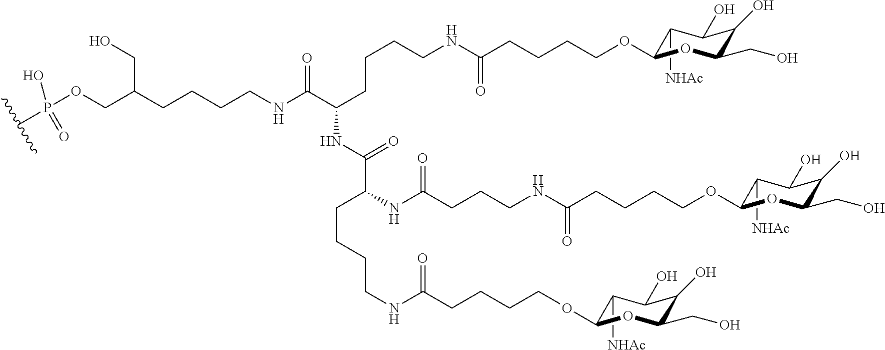

(12R, 15S)-1-(((2R,3R,4R,6R)-3-acetamido-4,5-dihydroxy-6-(hydroxymethyl)tetrahydro-2H-pyran-2-
yl)oxy)-12,15-bis(4-(5-(((2R,3R,4R,6R)-3-acetamido-4,5-dihydroxy-6-(hydroxymethyl)tetrahydro-2H-
pyran-2-yl)oxy)pentanamido)butyl)-22-(hydroxymethyl)-5,10,13,16-tetraoxo-6,11,14,17-
tetraazatricosan-23-yl hydroxyphosphoryl
Functionalized macromolecule name:
(12R,15S)-1-(((2R,3R,4R,6R)-3-acetamido-4,5-dihydroxy-6-(hydroxymethyl)tetrahydro-2HI-pyran-2-
yl)oxy)-12,15-bis(4-(5-(((2R,3R,4R,6R)-3-acetamido-4,5-dihydroxy-6-(hydroxymethyl)tetrahydro-2H-
pyran-2-yl)oxy)pentanamido)butyl)-22-(hydroxymethyl)-5,10,13,16-tetraoxo-6,11,14,17-
tetraazatricosan-23-yl hydroxyphosphoryl-2′-deoxy-fluoro-adenylyl-(3′→5′)-2′-O-methyl-guanylyl-
(3′→5′)-2-deoxy-fluoro-guanylyl-(3′→5′)-2′-O-methyl-uridylyl-(3′→5′)-2′-deoxy-fluoro-adenylyl-
(3′→5′)-2′-O-methyl-guanylyl-(3′→5′)-2′-deoxy-fluoro-uridylyl-(3′→5′)-2′-O-methyl-uridylyl-
(3′→5′)-2-deoxy-fluoro-guanylyl-(3′→5′)-2′-O-methyl-uridylyl-(3′→5′)-2′-deoxy-fluoro-adenylyl-
(3′→5′)-2′-deoxy-fluoro-uridylyl-(3′→5′)-2-deoxy-fluoro-guanylyl-(3′→5′)-2′-O-methyl-adenylyl-
(3′→5′)-P(R,S)-2′-deoxy-fluoro-P-thiocytidylyl-(3′→5′)-P(R,S)-2′-O-methyl-P-thioguanosine

### Example 10: Synthesis of 3′-tri-2-acetamido-2-deoxy-D-galactopyranose di-peptidyl Modified Oligonucleotide

Solid support platform of CPG containing tri-N-acetylchondrosamine di-peptidyl (L-Lys-D-Lys) modification was synthesized according to the general procedure for synthesis of solid support platform. Then, it was used for the synthesis of functionalized oligonucleotide on solid support platform according to the general procedure for the synthesis of functionalized oligonucleotide. Finally, functionalized oligonucleotide containing 3′-tri-2-acetamido-2-deoxy-D-galactopyranose di-peptidyl modification was cleaved and deprotected, purified and quantified according to the general procedure for the post-synthetic process to provide the target compound.

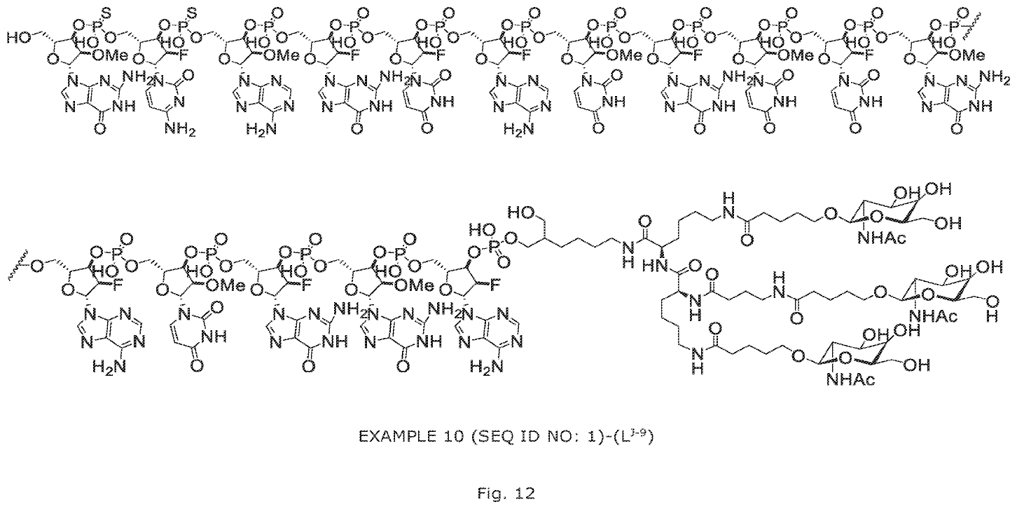

Solid support platform

| | |
|---|---|
| SSP Name | Tri-N-acetylchondrosamine di-peptidyl CPG |
| Molecular weight | Calculated 1701.0, Measured 1700.3 |
| Functionalized macromolecule structure shown in Fig. 12 | |
| (SEQ ID NO: 1)-(L^{J-9}) | 5′-HO-mG*fC*mAfGfUfAmUfGmUfUmGfAmUfGmGfA-3′-(L^{J-9}) |
| Sequence Name | 3′-Tri-2-acetamido-2-deoxy-D-galactopyranose di-peptidyl modified |
| | oligonucleotide |
| Molecular weight | Calculated 6770.9, Measured 6771.0 |

L^{J-9}: (GAL)-(GABA)-K^{L}(GAL)-K^{D}(GAL)-

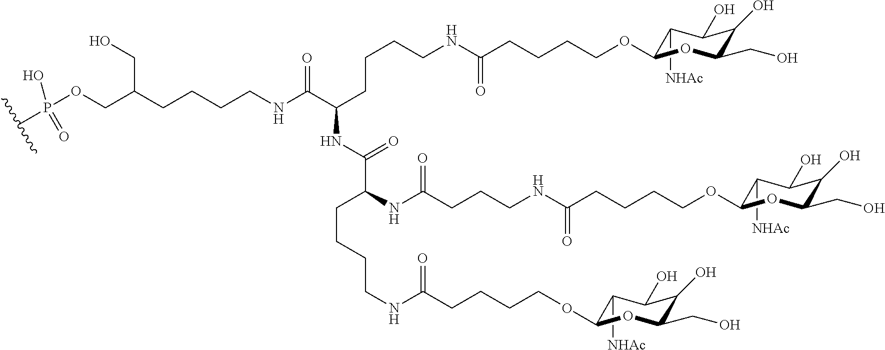

(12S,15R)-1-(((2R,3R,4R,6R)-3-acetamido-4,5-dihydroxy-6-(hydroxymethyl)tetrahydro-2H-pyran-2-
yl)oxy)-12,15-bis(4-(5-(((2R,3R,4R,6R)-3-acetamido-4,5-dihydroxy-6-(hydroxymethyl)tetrahydro-2H-
pyran-2-yl)oxy)pentanamido)butyl)-22-(hydroxymethyl)-5,10,13,16-tetraoxo-6,11,14,17-
tetraazatricosan-23-yl hydroxyphosphoryl
Functionalized macromolecule name:
(12S,15R)-1-(((2R,3R,4R,6R)-3-acetamido-4,5-dihydroxy-6-(hydroxymethyl)tetrahydro-2H-pyran-2-
yl)oxy)-12,15-bis(4-(5-(((2R,3R,4R,6R)-3-acetamido-4,5-dihydroxy-6-(hydroxymethyl)tetrahydro-2H-
pyran-2-yl)oxy)pentanamido)butyl)-22-(hydroxymethyl)-5,10,13,16-tetraoxo-6,11,14,17-
tetraazatricosan-23-yl hydroxyphosphoryl-2′-deoxy-fluoro-adenylyl-(3′→5′)-2′-O-methyl-guanylyl-
(3′→5′)-2-deoxy-fluoro-guanylyl-(3′→5′)-2′-O-methyl-uridylyl-(3′→5′)-2′-deoxy-fluoro-adenylyl-
(3′→5′)-2′-O-methyl-guanylyl-(3′→5′)-2′-deoxy-fluoro-uridylyl-(3′→5′)-2′-O-methyl-uridylyl-
(3′→5′)-2-deoxy-fluoro-guanylyl-(3′→5′)-2′-O-methyl-uridylyl-(3′→5′)-2′-deoxy-fluoro-adenylyl-
(3′→5′)-2′-deoxy-fluoro-uridylyl-(3′→5′)-2-deoxy-fluoro-guanylyl-(3′→5′)-2′-O-methyl-adenylyl-
(3′→5′)-P(R,S)-2′-deoxy-fluoro-P-thiocytidylyl-(3′→5′)-P(R,S)-2′-O-methyl-P-thioguanosine

### Example 11: Synthesis of 3′-tri-2-acetamido-2-deoxy-D-galactopyranose di-peptidyl Modified Oligonucleotide

Solid support platform of CPG containing tri-N-acetylchondrosamine di-peptidyl (D-Lys-D-Lys) modification was synthesized according to the general procedure for synthesis of solid support platform. Then, it was used for the synthesis of functionalized oligonucleotide on solid support platform according to the general procedure for the synthesis of functionalized oligonucleotide. Finally, functionalized oligonucleotide containing 3′-tri-2-acetamido-2-deoxy-D-galactopyranose di-peptidyl modification was cleaved and deprotected, purified and quantified according to the general procedure for the post-synthetic process to provide the target compound.

Solid support platform

| | |
|---|---|
| SSP Name | Tri-N-acetylchondrosamine di-peptidyl CPG |
| Molecular weight | Calculated 1701.0, Measured 1700.3 |

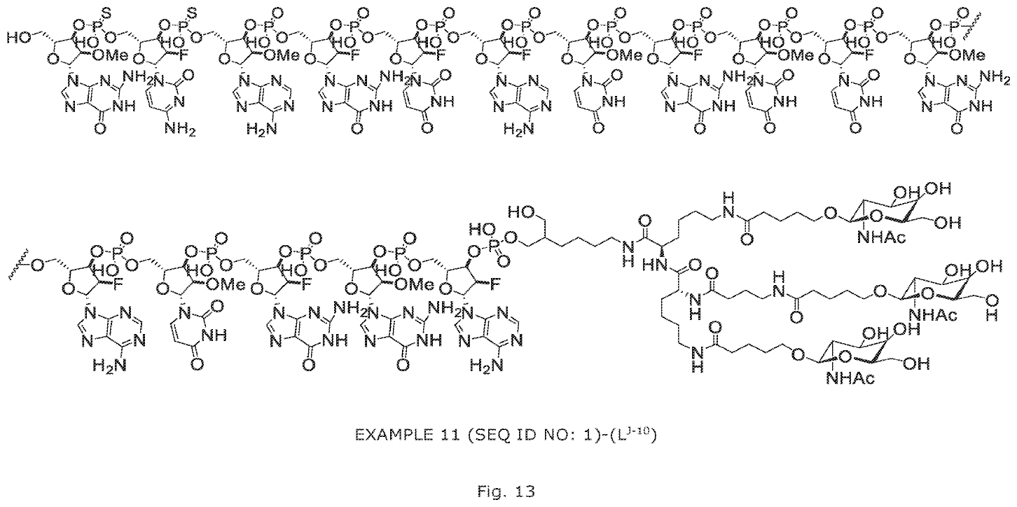

Functionalized macromolecule structure shown in Fig. 13

| | |
|---|---|
| (SEQ ID NO: 1)-(L^{J-10}) | 5′-HO-mG*fC*mAfGfUfAmUfGmUfUmGfAmUfGmGfA-3′-(L^{J-10}) |
| Sequence Name | 3′-Tri-2-acetamido-2-deoxy-D-galactopyranose di-peptidyl modified |
| | oligonucleotide |
| Molecular weight | Calculated 6770.9, Measured 6771.0 |

L^{J-10}: (GAL)-(GABA)-K^{D}(GAL)-K^{D}(GAL)-

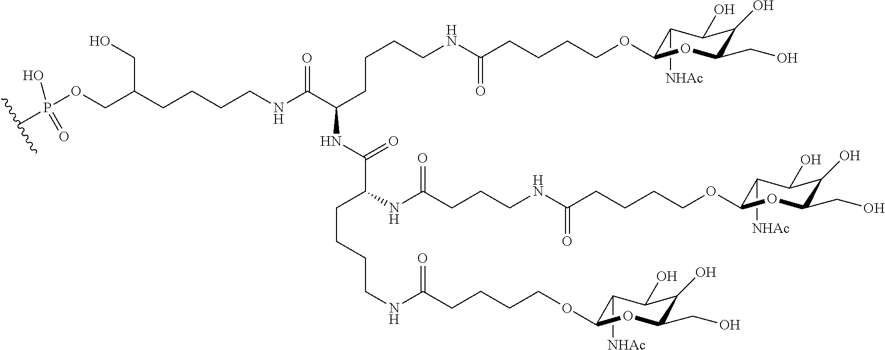

(12R, 15R)-1-(((2R,3R,4R,6R)-3-acetamido-4,5-dihydroxy-6-(hydroxymethyl)tetrahydro-2H-pyran-2-
yl)oxy)-12,15-bis(4-(5-(((2R,3R,4R,6R)-3-acetamido-4,5-dihydroxy-6-(hydroxymethyl)tetrahydro-2H-
pyran-2-yl)oxy)pentanamido)butyl)-22-(hydroxymethyl)-5,10,13,16-tetraoxo-6,11,14,17-
tetraazatricosan-23-yl hydroxyphosphoryl
Functionalized macromolecule name:
(12R,15R)-1-(((2R,3R,4R,6R)-3-acetamido-4,5-dihydroxy-6-(hydroxymethyl)tetrahydro-2H-pyran-2-
yl)oxy)-12,15-bis(4-(5-(((2R,3R,4R,6R)-3-acetamido-4,5-dihydroxy-6-(hydroxymethyl)tetrahydro-2H-
pyran-2-yl)oxy)pentanamido)butyl)-22-(hydroxymethyl)-5,10, 13,16-tetraoxo-6,11,14,17-
tetraazatricosan-23-yl hydroxyphosphoryl-2′-deoxy-fluoro-adenylyl-(3′→5′)-2′-O-methyl-guanylyl-
(3′→5′)-2-deoxy-fluoro-guanylyl-(3′→5′)-2′-O-methyl-uridylyl-(3′→5′)-2′-deoxy-fluoro-adenylyl-
(3′→5′)-2′-O-methyl-guanylyl-(3′→5′)-2′-deoxy-fluoro-uridylyl-(3′→5′)-2′-O-methyl-uridylyl-
(3′→5′)-2-deoxy-fluoro-guanylyl-(3′→5′)-2′-O-methyl-uridylyl-(3′→5′)-2′-deoxy-fluoro-adenylyl-
(3′→5′)-2′-deoxy-fluoro-uridylyl-(3′→5′)-2-deoxy-fluoro-guanylyl-(3′→5′)-2′-O-methyl-adenylyl-
(3′→5′)-P(R,S)-2′-deoxy-fluoro-P-thiocytidylyl-(3′→5′)-P(R,S)-2′-O-methyl-P-thioguanosine

### Example 12: Synthesis of 3′-tri-2-acetamido-2-deoxy-D-galactopyranose tetra-peptidyl Modified Oligonucleotide Using tri-N-acetylchondrosamine tetra-peptidyl Silica Gel (CPSG: Controlled Pore Silica Gel)

Solid support platform of PS containing 3′-tri-2-acetamido-2-deoxy-D-galactopyranose tetra-peptidyl modification was synthesized according to the general procedure for synthesis of solid support platform. Then, it was used for the synthesis of functionalized oligonucleotide on solid support platform according to the general procedure for the synthesis of functionalized oligonucleotide. Finally, functionalized oligonucleotide containing 3′-tri-2-acetamido-2-deoxy-D-galactopyranose tetra-peptidyl modification was cleaved and deprotected, purified and quantified according to the general procedure for the post-synthetic process to provide the target compound.

Solid support platform

| | |
|---|---|
| SSP Name | Tri-N-acetylchondrosamine tetra-peptidyl silica gel (CPSG; controlled pore |
| | silica gel) |
| Molecular weight | Calculated 1847.0, Measured 1847.0 (after cleavage from solid support) |

Functionalized macromolecule structure shown in FIG. 4

| | |
|---|---|
| (SEQ ID NO: 1)-(L^{J-1}) | 5′-HO-mG*fC*mAfGfUfAmUfGmUfUmGfAmUfGmGfA-3′-(L^{J-1}) |
| Sequence Name | 3′-Tri-2-acetamido-2-deoxy-D-galactopyranose tetra-peptidyl modified |
| | oligonucleotide |
| Molecular weight | Calculated 6918.1, Measured 6917.8 |

### Example 13: Synthesis of 3′-tri-2-acetamido-2-deoxy-D-galactopyranose tetra-peptidyl Modified Oligonucleotide Using tri-N-acetylchondrosamine tetra-peptidyl Hybrid Resin (NittoPhase or NittoPhase HL)

Solid support platform of PS containing 3′-tri-2-acetamido-2-deoxy-D-galactopyranose tetra-peptidyl modification was synthesized according to the general procedure for synthesis of solid support platform. Then, it was used for the synthesis of functionalized oligonucleotide on solid support platform according to the general procedure for the synthesis of functionalized oligonucleotide. Finally, functionalized oligonucleotide containing 3′-tri-2-acetamido-2-deoxy-D-galactopyranose tetra-peptidyl modification was cleaved and deprotected, purified and quantified according to the general procedure for the post-synthetic process to provide the target compound.

Solid support platform

| | |
|---|---|
| SSP Name | Tri-N-acetylchondrosamine tetra-peptidyl hybrid resin (NittoPhase or |
| | NittoPhase HL) |
| Molecular weight | Calculated 1847.0, Measured 1847.0 (after cleavage from solid support) |

Functionalized macromolecule structure shown in FIG. 4

| | |
|---|---|
| (SEQ ID NO: 1)-(L^{J-1}) | 5′-HO-mG*fC*mAfGfUfAmUfGmUfUmGfAmUfGmGfA-3′-(L^{J-1}) |
| Sequence Name | 3′-Tri-2-acetamido-2-deoxy-D-galactopyranose tetra-peptidyl modified |
| | oligonucleotide |
| Molecular weight | Calculated 6918.1, Measured 6917.8 |

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value, according to the specified significant figures, falling within the range. For example, a range of 1-3, may be a shorthand way of referring to 1-2-3, which in some embodiments may also be referred to as 1, 2, or 3. Similarly, 1.1-1.4 may refer to 1.1-1.2-1.3-1.4, or 1.1, 1.2, 1.3, or 1.4 in some embodiments. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for this disclosure to be practiced otherwise than only in the manner specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Thus, by way of example, but not of limitation, alternative configurations of the present disclosure may be utilized in accordance with the teachings herein. Accordingly, the present disclosure is not limited to that precisely as shown and described.

1. A compound, comprising one or more of the formulae: 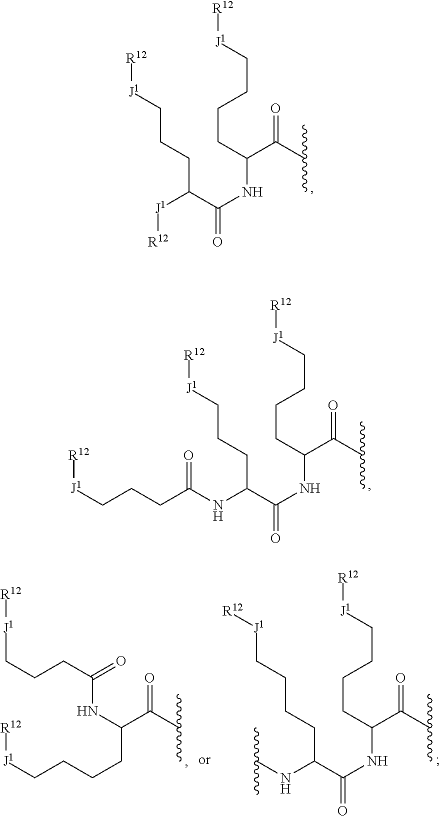
or a salt thereof,
wherein
each J¹ is, independently, N(H) or C(O);
each R¹² is, independently, selected from H, OH, —C(O)(CH₂)_{z}-(ligand), —N(H)(CH₂)_{z}-(ligand), or —O—(CH₂)_{z}-(ligand);
each z is, independently, selected from 1, 2, 3, 4, 5, or 6; and
each ligand is, independently, selected from a lipophilic group, a protein, a peptide, an oligosaccharide, a nucleic acid, a polymer, a carbohydrate, or a lipid.

2. The compound of claim 1, wherein each z is, independently, 3 or 4, and each ligand is, independently, a mannose moiety, an N-acetylated galactosamine moiety, a tetra-acetylated mannose moiety or a tetra-acetylated galactosamine moiety.

3. The compound of claim 1, wherein the compound is a macromolecule functionalized with one or more of the formulae.

4. The compound of claim 1, comprising a macromolecule covalently linked to one or more, independently, of the formula: 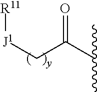
or a salt thereof,
wherein
R¹¹ is selected from H, OH, —C(O)(CH₂)_{z}-(ligand), —N(H)(CH₂)_{z}-(ligand), or —O—(CH₂)_{z}-(ligand), 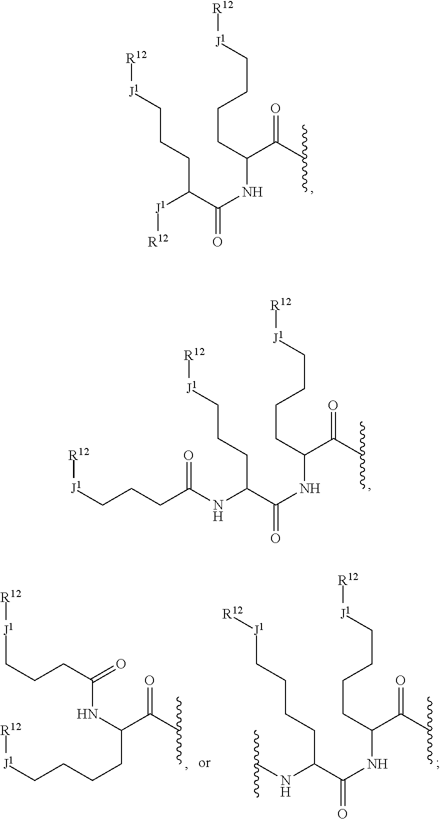
and
each y is, independently, selected from 1, 2, 3, 4, 5, or 6.

5. The compound of claim 1, comprising a macromolecule covalently linked to one or more, independently, of the formula: 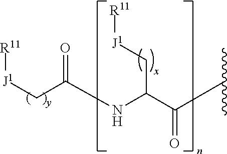
or a salt thereof,
wherein
each R¹¹ is, independently, selected from H, OH, —C(O)(CH₂)_{z}-(ligand), —N(H)(CH₂)_{z}-(ligand), or —O—(CH₂)_{z}-(ligand), 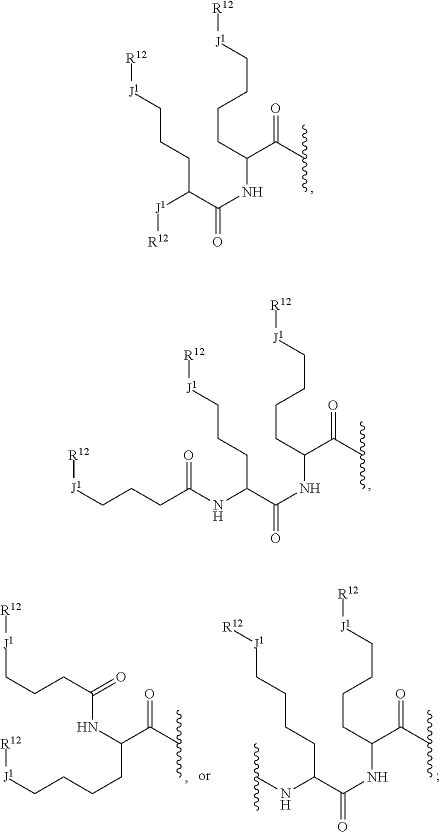
each x is, independently, selected from 1, 2, 3, 4, 5, or 6;
each y is, independently, selected from 1, 2, 3, 4, 5, or 6; and
n is 0, 1, 2, 3, 4, 5, or 6.

6. The compound of claim 1, comprising a macromolecule covalently linked to one or more, independently, of the formula: 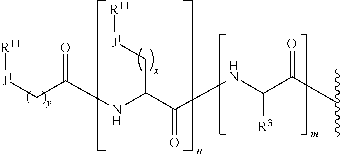
or a salt thereof,
wherein
R³ is —(C₀₋₃ alkylene)-(C₃₋₈ cycloalkyl), —(C₀₋₃ alkylene)-(C₃₋₈ heterocycloalkyl), —(C₁₋₃ alkylene)-(C₆₋₁₀ aryl), —(C₁₋₃ alkylene)-(C₂₋₁₀ heteroaryl), wherein the aryl or heteroaryl is optionally substituted by one or two functional groups selected, independently, from OH, F, Cl, Br, I, O—C₁₋₃ alkyl, C₁₋₃ alkyl, C₁₋₃ alkenyl, C₁₋₃ alkynyl, C₁₋₃ haloalkyl, COOH, or NH₂;
each R¹¹ is, independently, selected from H, OH, —C(O)(CH₂)_{z}-(ligand), —N(H)(CH₂)_{z}-(ligand), or —O—(CH₂)_{z}-(ligand), 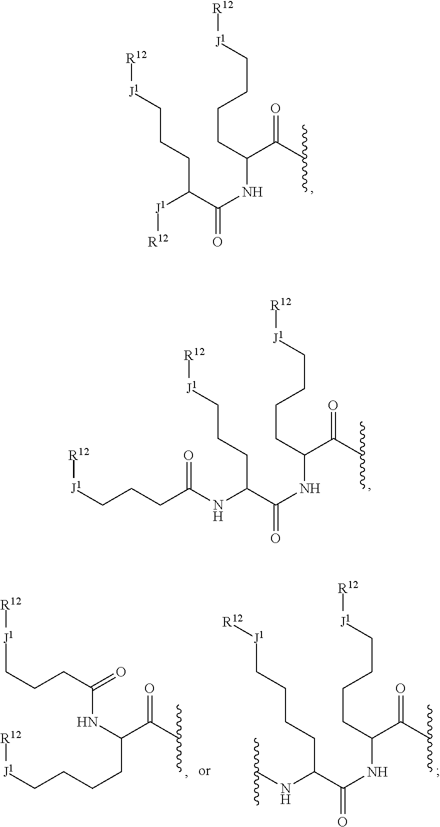
each x is, independently, selected from 1, 2, 3, 4, 5, or 6;
each y is, independently, selected from 1, 2, 3, 4, 5, or 6;
m is 0, 1, 2, 3, 4, 5, or 6; and
n is 0, 1, 2, 3, 4, 5, or 6.

7. The compound of claim 1, comprising a macromolecule covalently linked to one or more, independently, of the formula: 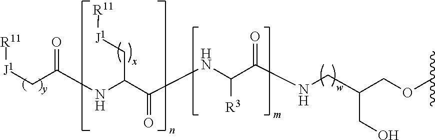
or a salt thereof,
wherein
R³ is —(C₀₋₃ alkylene)-(C₃₋₈ cycloalkyl), —(C₀₋₃ alkylene)-(C₃₋₈ heterocycloalkyl), —(C₁₋₃ alkylene)-(C₆₋₁₀ aryl), —(C₁₋₃ alkylene)-(C₂₋₁₀ heteroaryl), wherein the aryl or heteroaryl is optionally substituted by one or two functional groups selected, independently, from OH, F, Cl, Br, I, O—C₁₋₃ alkyl, C₁₋₃ alkyl, C₁₋₃ alkenyl, C₁₋₃ alkynyl, C₁₋₃ haloalkyl, COOH, or NH₂;
each R¹¹ is, independently, selected from H, OH, —C(O)(CH₂)_{z}-(ligand), —N(H)(CH₂)_{z}-(ligand), or —O—(CH₂)_{z}-(ligand), 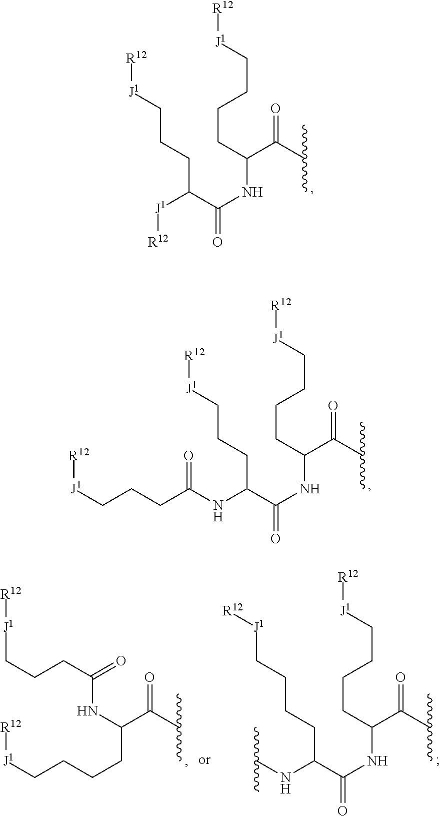
each x is, independently, selected from 1, 2, 3, 4, 5, or 6;
each y is, independently, selected from 1, 2, 3, 4, 5, or 6;
each w is, independently, selected from 1, 2, 3, 4, 5, or 6;
m is 0, 1, 2, 3, 4, 5, or 6; and
n is 0, 1, 2, 3, 4, 5, or 6.

8. The compound of claim 7, comprising the formula: 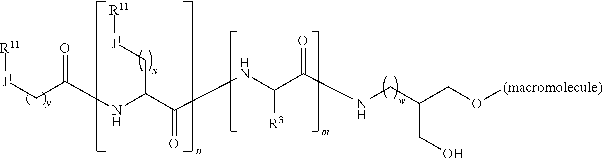
or a salt thereof.

9. The compound of claim 1, comprising a macromolecule covalently linked to one or more, independently, of the formula: 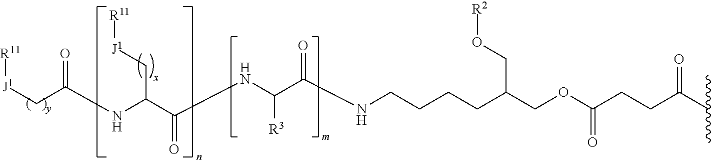
or a salt thereof,
wherein
R² is a thermolytic, photolytic, tris(2-carboxyethyl) phosphine-labile, acid-labile, or base-labile moiety;
R³ is —(C₀₋₃ alkylene)-(C₃₋₈ cycloalkyl), —(C₀₋₃ alkylene)-(C₃₋₈ heterocycloalkyl), —(C₁₋₃ alkylene)-(C₆₋₁₀ aryl), —(C₁₋₃ alkylene)-(C₂₋₁₀ heteroaryl), wherein the aryl or heteroaryl is optionally substituted by one or two functional groups selected, independently, from OH, F, Cl, Br, I, O—C₁₋₃ alkyl, C₁₋₃ alkyl, C₁₋₃ alkenyl, C₁₋₃ alkynyl, C₁₋₃ haloalkyl, COOH, or NH₂;
each R¹¹ is, independently, selected from H, OH, —C(O)(CH₂)_{z}-(ligand), —N(H)(CH₂)_{z}-(ligand), or —O—(CH₂)_{z}-(ligand), 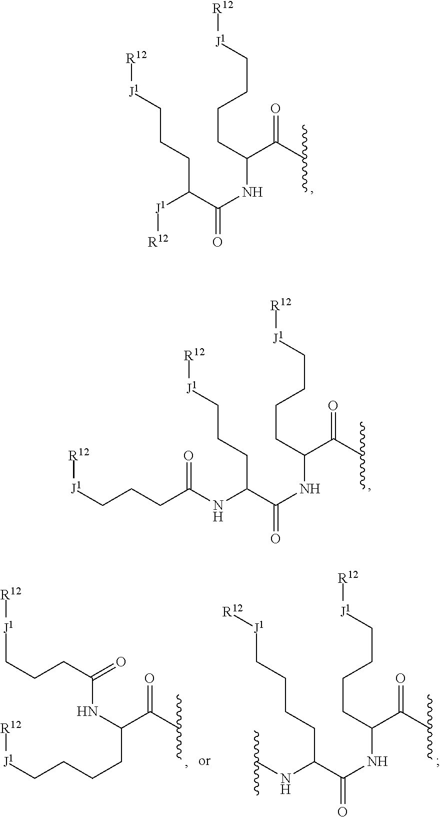
each x is, independently, selected from 1, 2, 3, 4, 5, or 6;
each y is, independently, selected from 1, 2, 3, 4, 5, or 6;
m is 0, 1, 2, 3, 4, 5, or 6; and
n is 0, 1, 2, 3, 4, 5, or 6.

10. The compound of claim 9, comprising the formula: 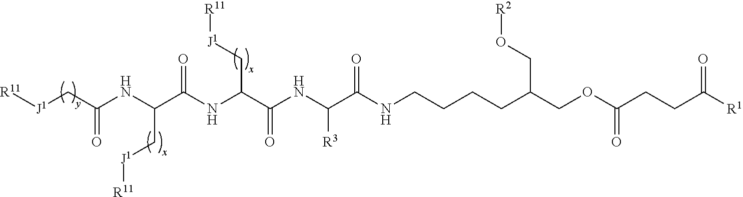
or a salt thereof,
wherein
R¹ is a solid support.

11. The compound of claim 9, comprising the formula: 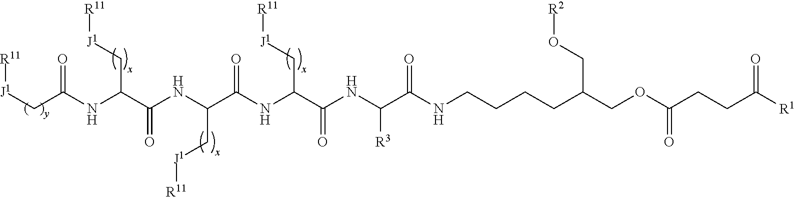
or a salt thereof,
wherein
R¹ is a solid support.

12. The compound of claim 4, wherein the macromolecule is a peptide, a protein, an oligosaccharide, an oligonucleotide, or a solid support.

13. The compound of claim 4, wherein each x is 3 or 4, and each n is 2 or 3.

14. The compound of claim 4, wherein each R¹¹ and R¹² is H.

15. The compound of claim 4, wherein each ligand is, independently, selected from a mannose moiety, an N-acetylated galactosamine moiety, a tetra-acetylated mannose moiety or a tetra-acetylated galactosamine moiety.

16. The compound of claim 4, wherein each x is the same, and each ligand is the same.

17. The compound of claim 1, wherein each z is 4.

18. A compound of claim 1, comprising one of the formulae selected from: 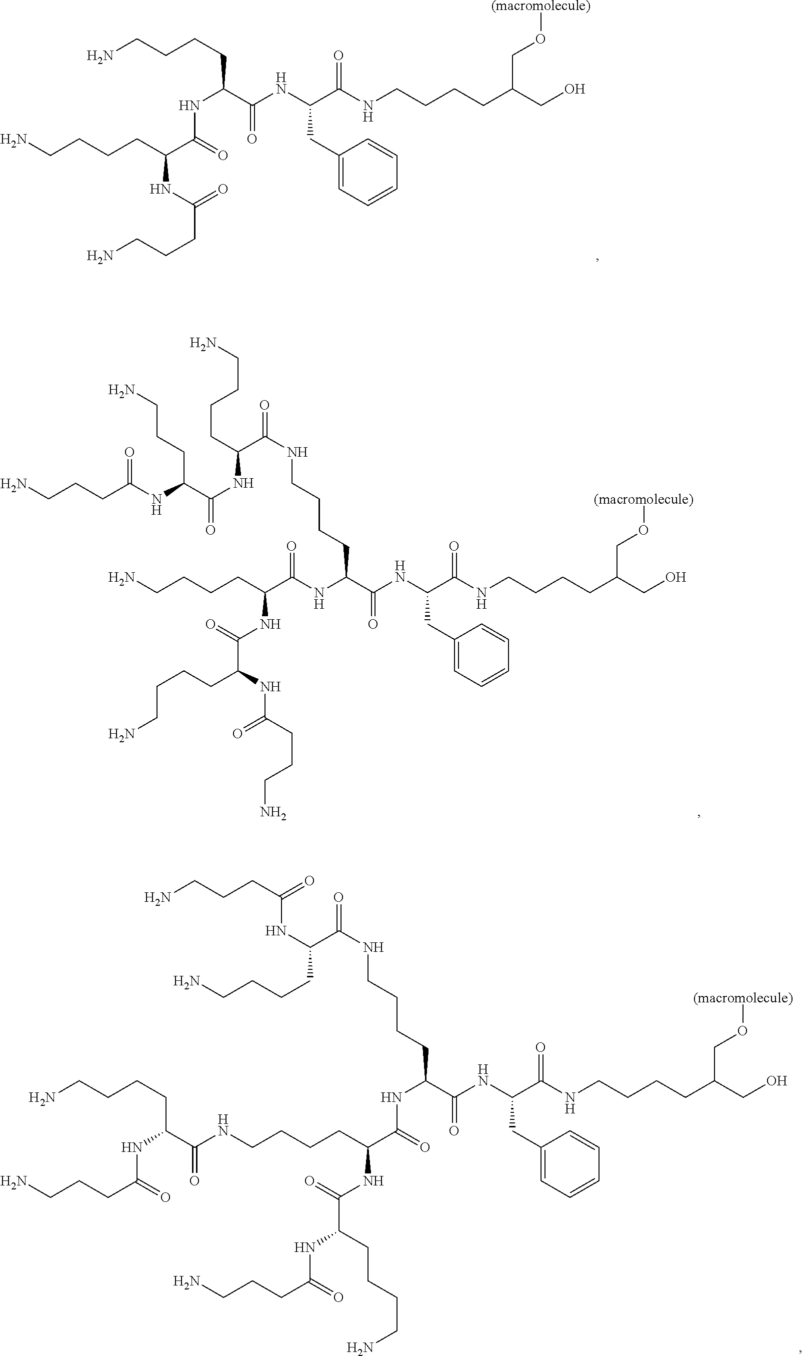 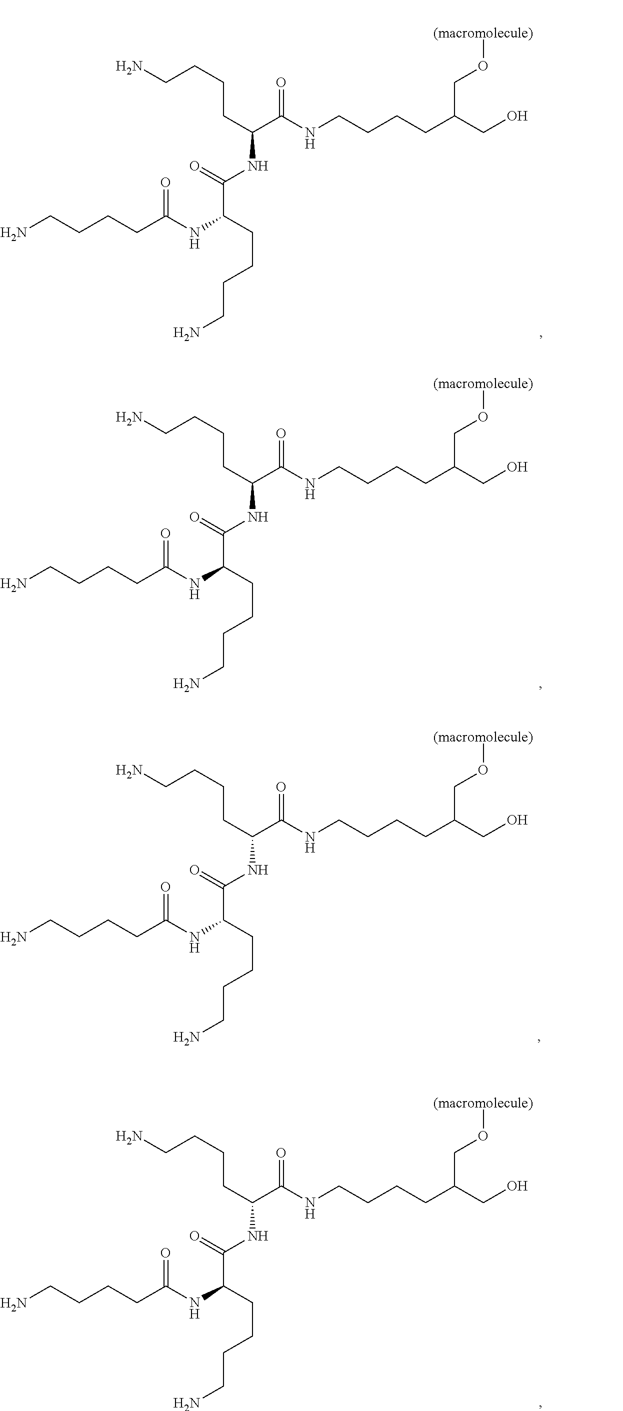 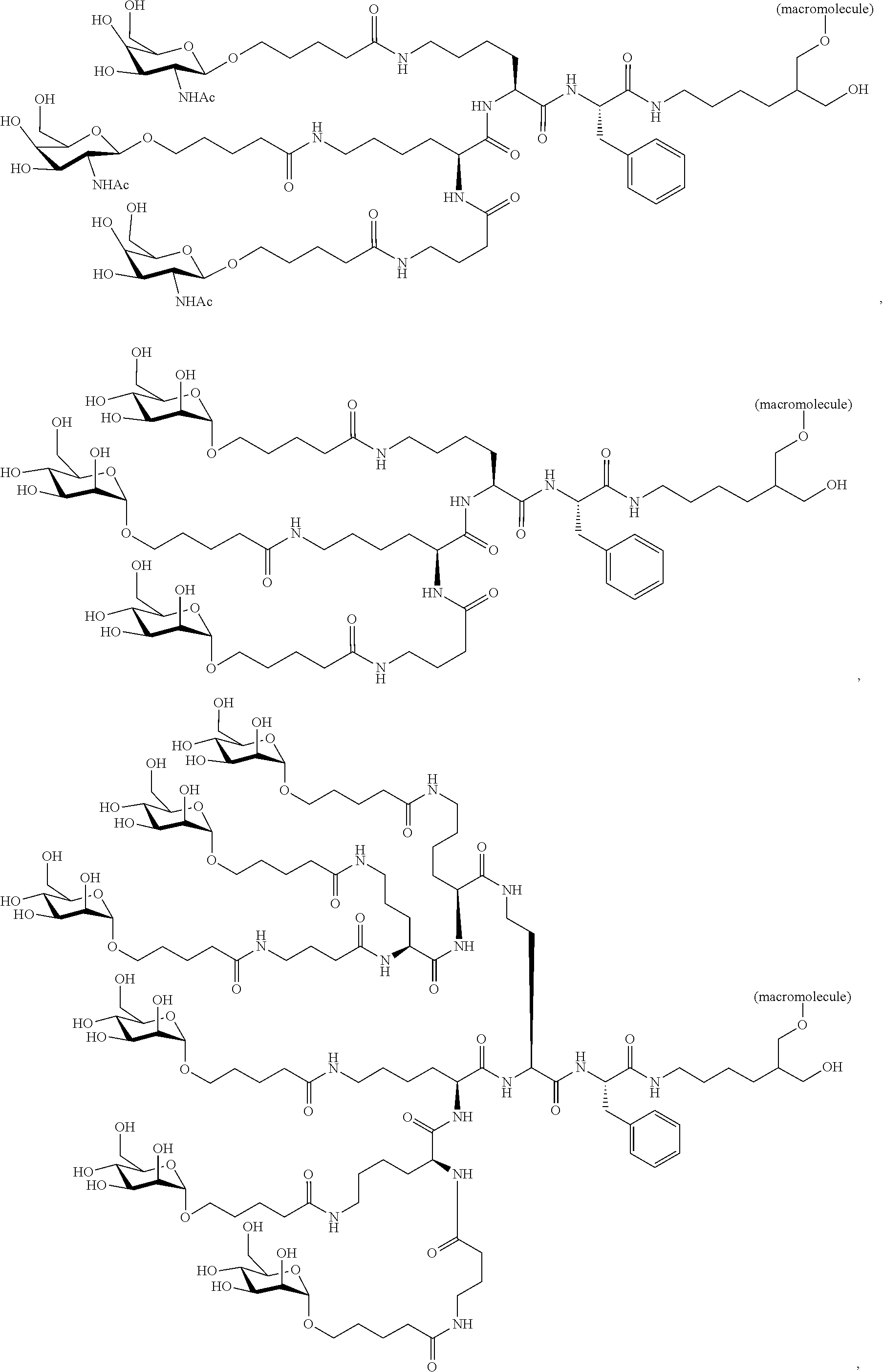 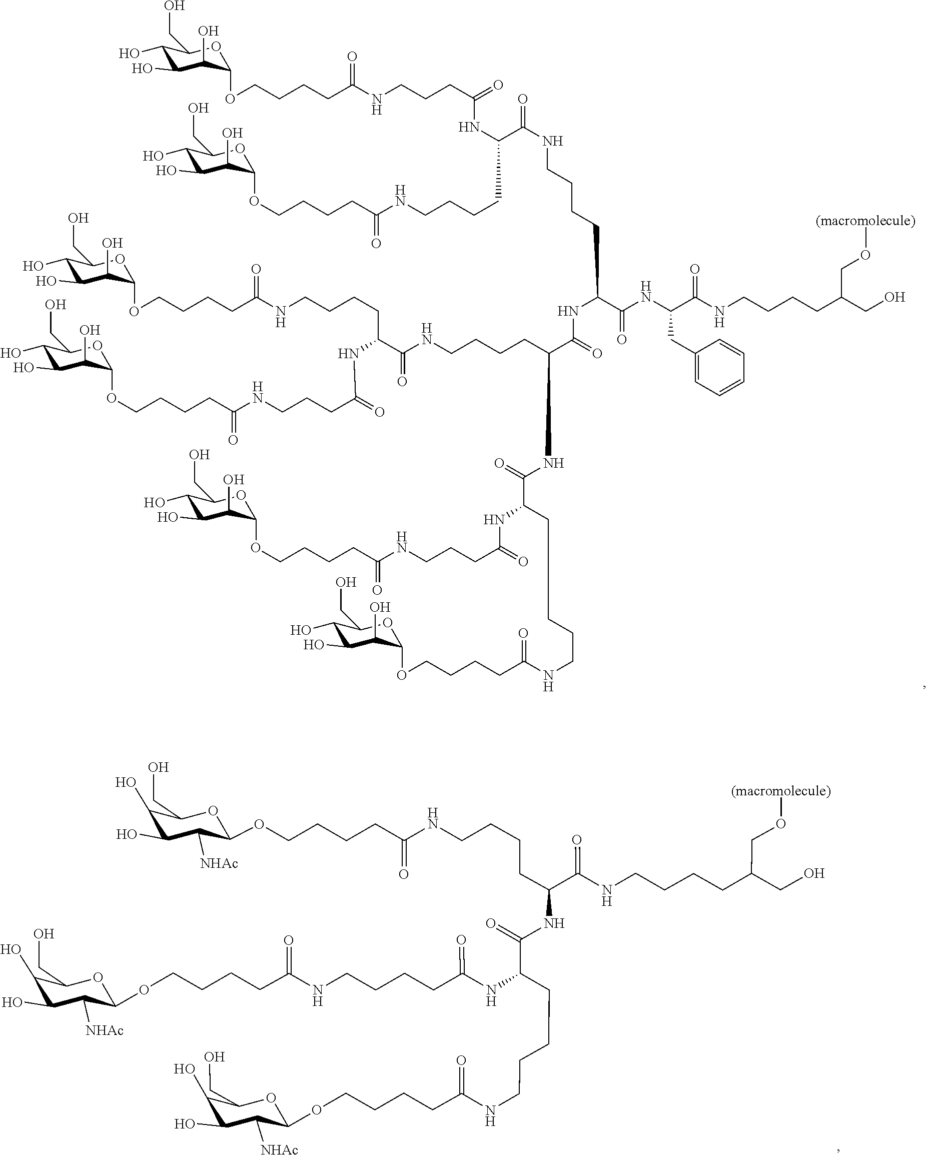 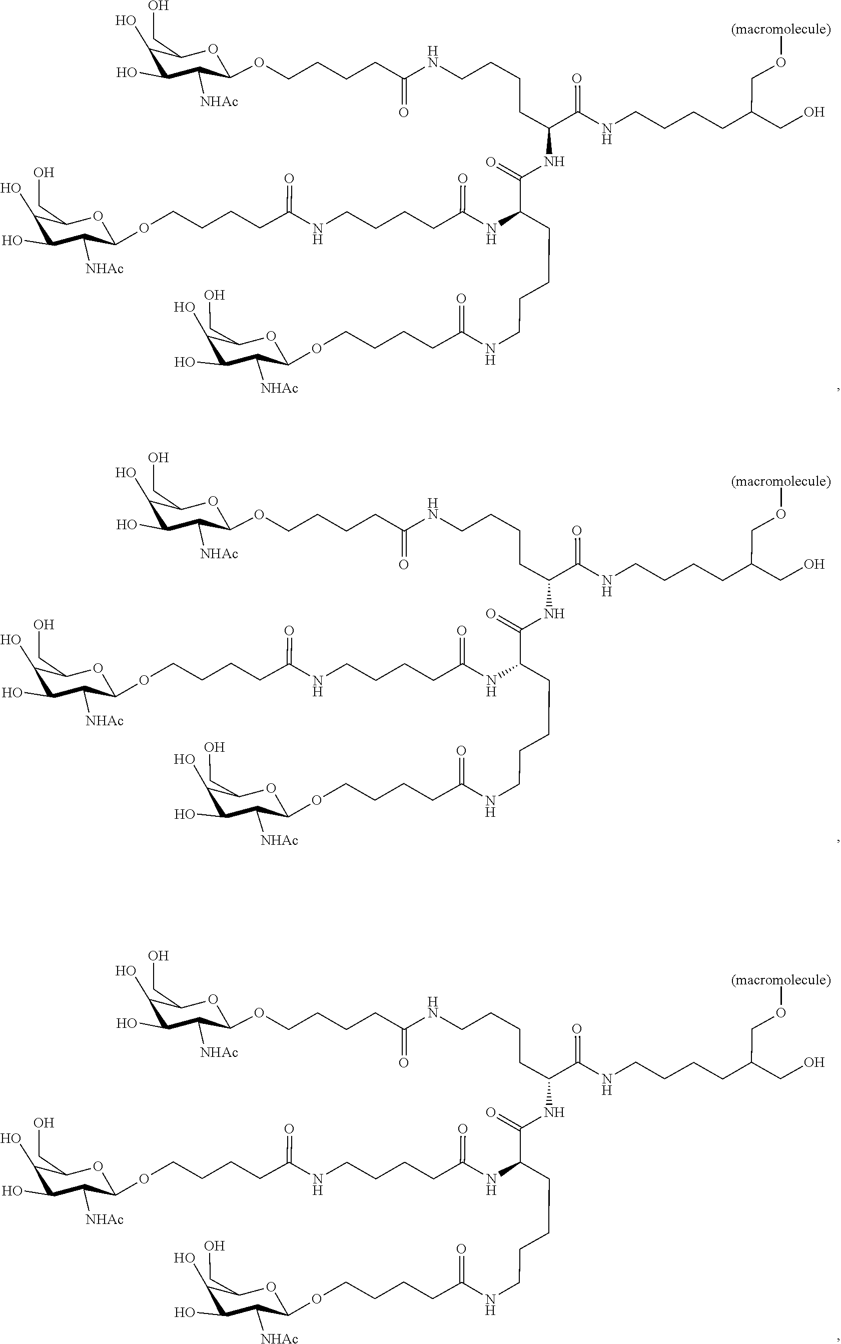 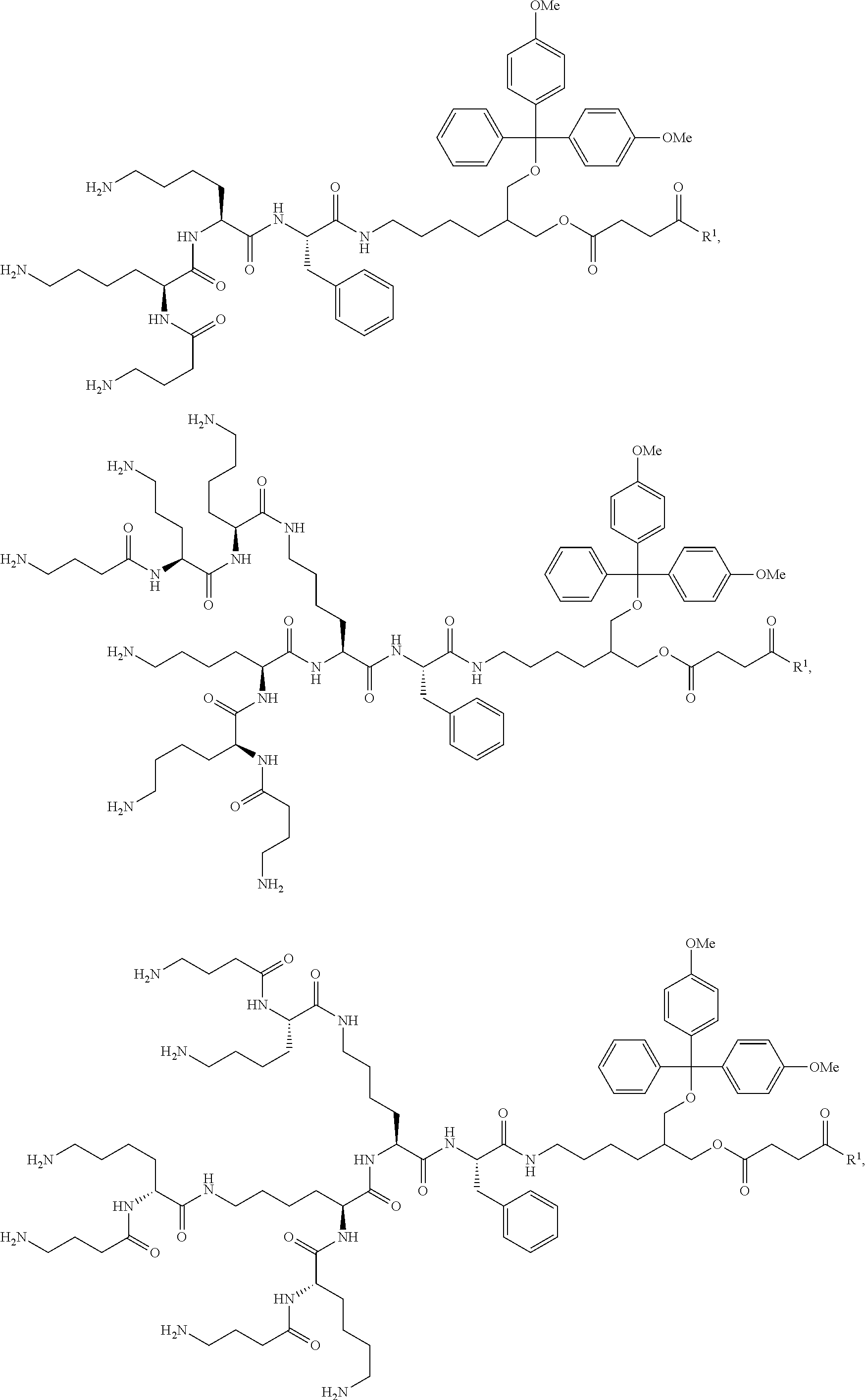 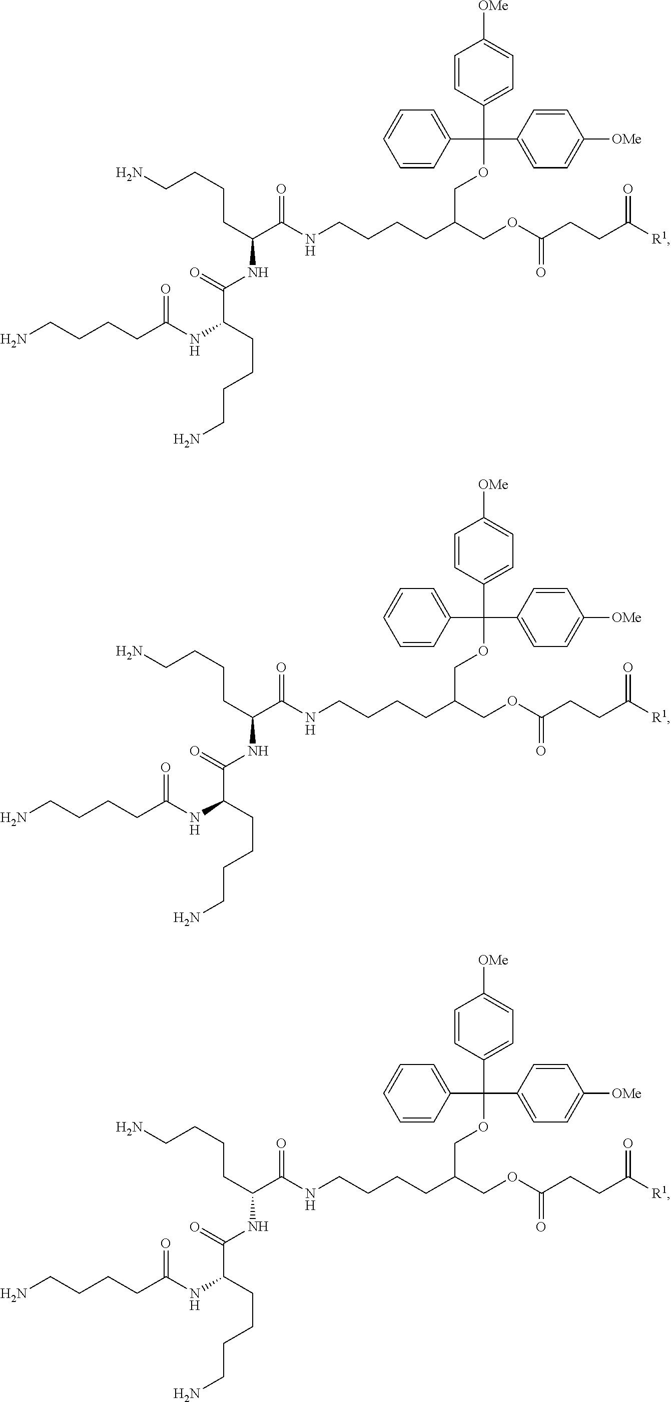 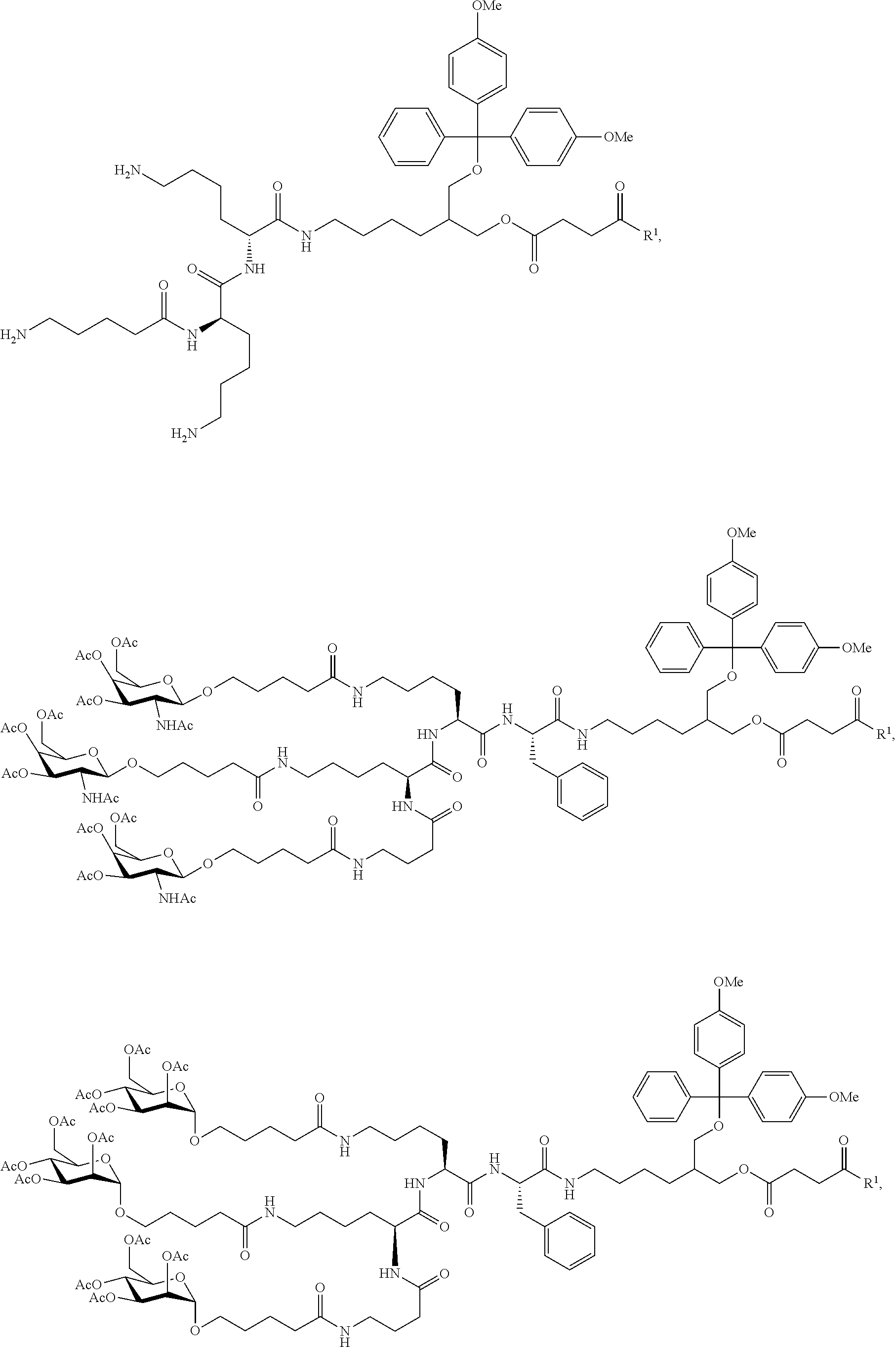 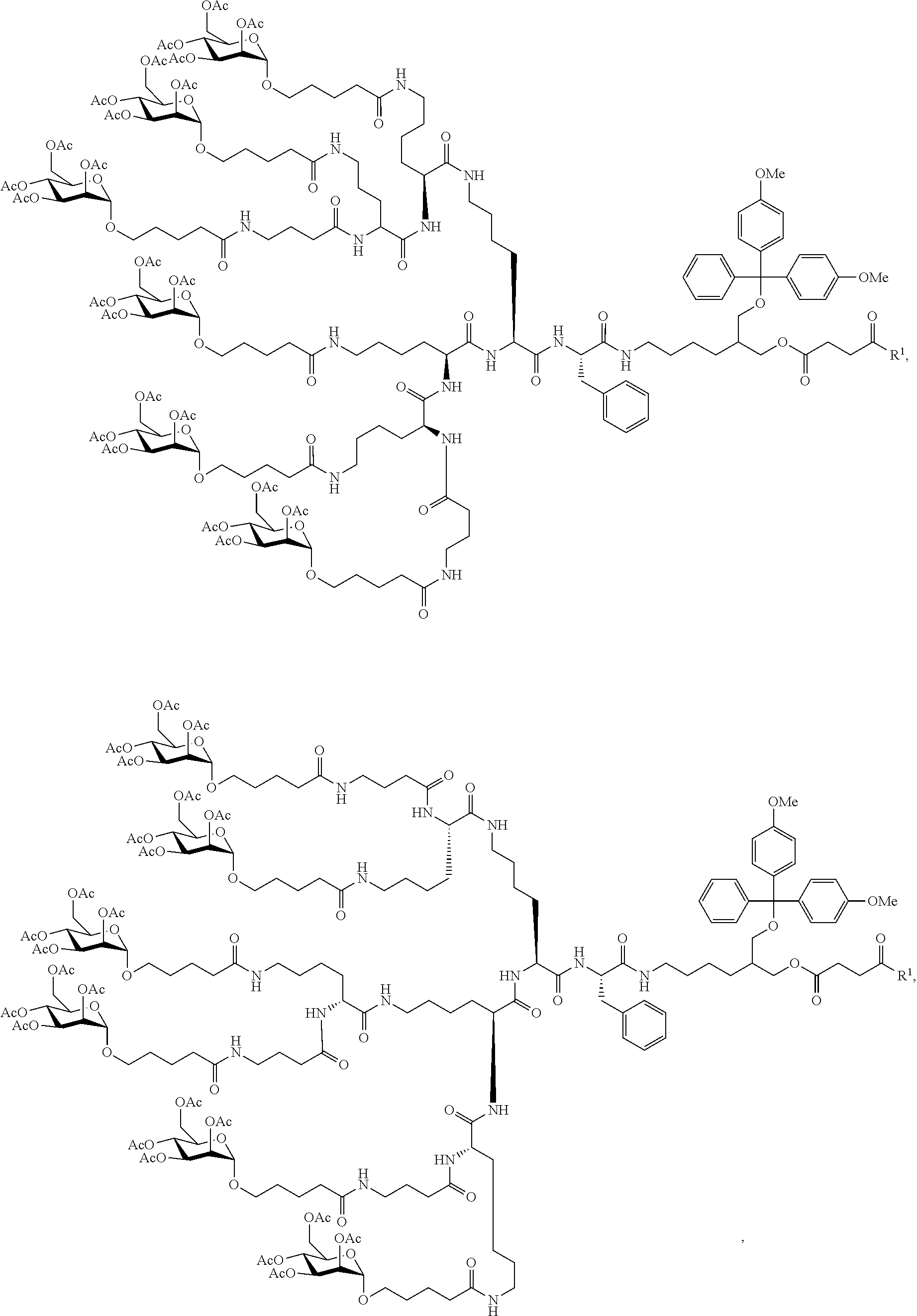 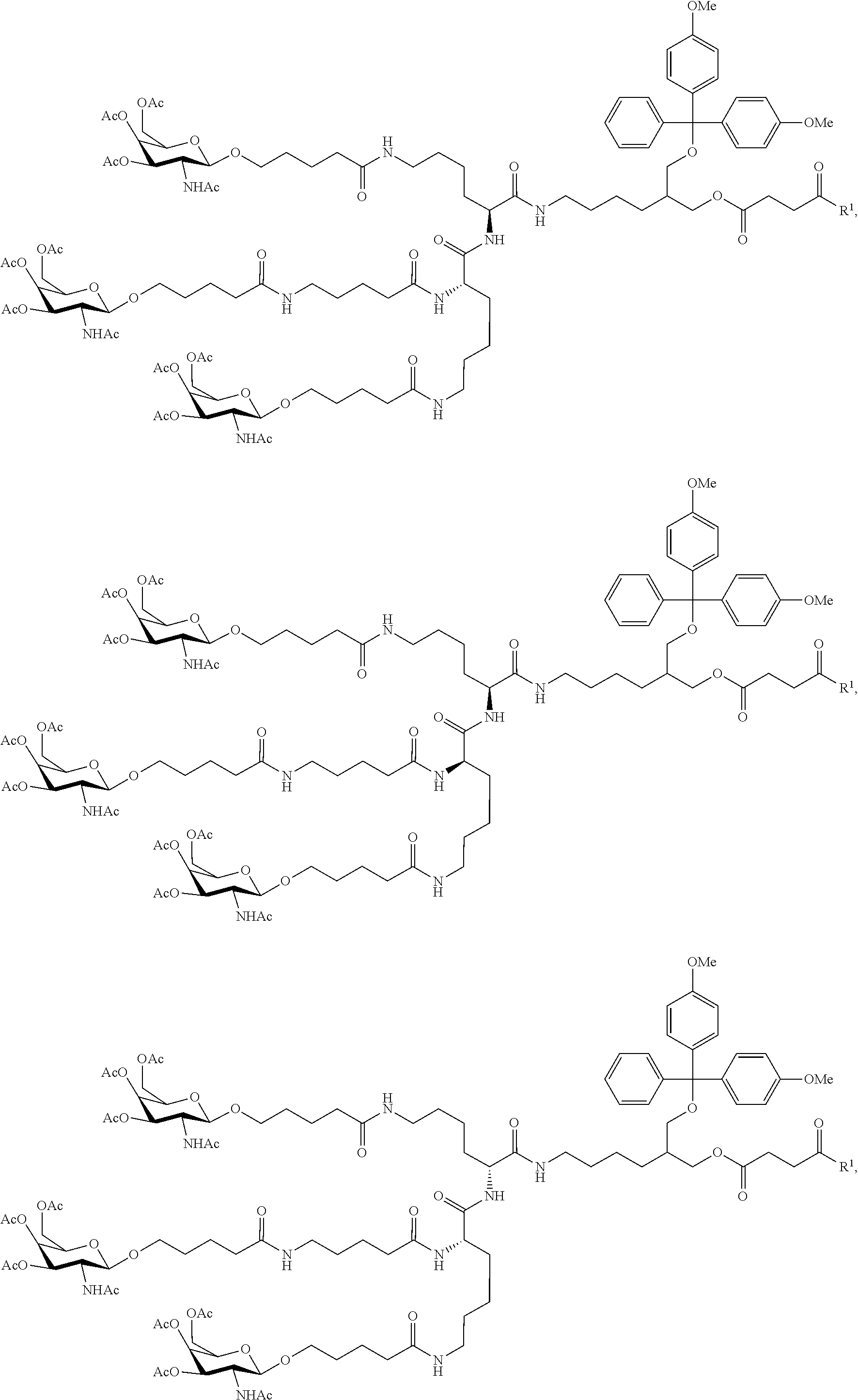 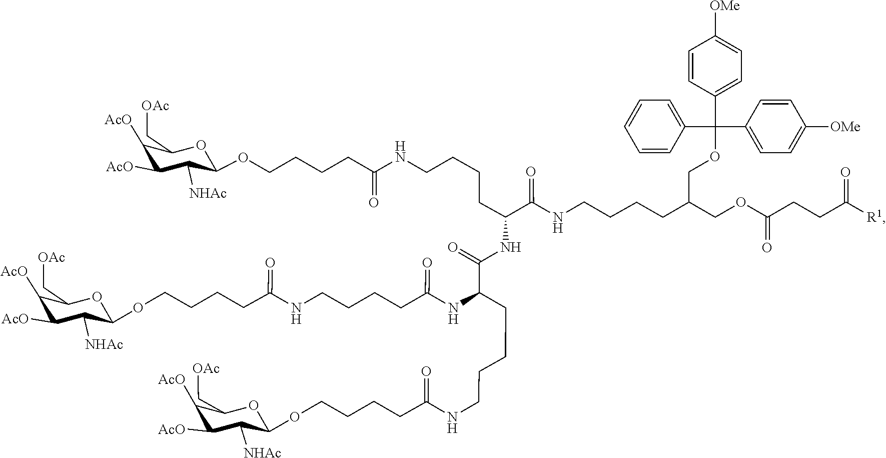
or a salt thereof,
wherein
R¹ is a solid support, and
the macromolecule is a peptide, a protein, an oligosaccharide, an oligonucleotide, or a solid support.

19-33. (canceled)

34. A composition, comprising the compound of claim 18, optionally wherein the composition is a pharmaceutical composition further comprising a pharmaceutically acceptable carrier.

35. A method of activating gene silencing in a cell, comprising contacting the cell with an effective amount of the compound of claim 18, wherein the compound comprises an oligonucleotide.

36. (canceled)

37. (canceled)
